(19) United States
(12) Patent Application Publication
(10) Pub. No.: US 2012/0004116 A1
(43) Pub. Date: Jan. 5, 2012

(54) METHODS FOR BIOMARKER IDENTIFICATION AND BIOMARKER FOR NON-SMALL CELL LUNG CANCER

(76) Inventors: Ming-Sound Tsao, Toronto (CA); Paul C. Boutros, Toronto (CA); Suzanne K. Lau, Willowdale (CA); Frances A. Shepperd, Toronto (CA); Linda Z. Penn, Toronto (CA); Igor Jurisica, Toronto (CA); Sandy D. Der, Toronto (CA)

(21) Appl. No.: 13/132,877

(22) PCT Filed: Dec. 2, 2009
(86) PCT No.: PCT/CA2009/001775

§ 371 Date: Sep. 26, 2011

Related U.S. Application Data
(60) Provisional application No. 61/119,936, filed on Dec. 4, 2008.
(60) Provisional application No. 61/149,847, filed on Feb. 4, 2009.

(51) Int. Cl. C12Q 1/68; C40B 30/00

(52) U.S. Cl. 506/7; 435/6.12

(57) ABSTRACT

There is provided a method for identifying a biomarker, such as a gene signature, associated with a biological parameter A 6-gene signature for non-small cell lung cancer (NSCLC) is also provided, as well as a method of prognosing or classifying a subject with non-small cell lung cancer into a poor survival group or a good survival group, using said gene signature

### FIELD OF THE INVENTION

The application relates generally to methods for biomarker identification and to biomarkers for non-small cell lung cancer.

### BACKGROUND OF THE INVENTION

Non-small cell lung cancer (NSCLC) is the predominant histological type of lung cancer, accounting for up to 85% of cases (1). Tumor stage is the best established and validated predictor of patient survival (2). When identified at an early stage, NSCLC is primarily treated by surgical resection, which is potentially curative. However 30-60% of patients with stage IB to IIIA NSCLC die within five years after surgery, primarily from tumor recurrence (3). These relapses have been postulated to arise from a reservoir of cells beyond the resection site, such as microscopic residual tumors at the resection margin, occult systemic metastases, or circulating tumor cells. Such a reservoir could potentially be eliminated with an adjuvant systemic therapy, such as systemic chemotherapy. Indeed, this type of adjuvant therapy is routinely applied in the treatment of other solid tumors, including breast (4) and colorectal cancer (5, 6).

Randomized clinical trials have confirmed the benefit of adjuvant chemotherapy in stage II to IIIA NSCLC patients, but the benefit in stage I remains controversial (7-10). However, even in stage I the overall survival is only 70%, which suggests that there is a sub-population of stage I patients who have more aggressive tumors. In theory these patients might benefit from post-operative adjuvant chemotherapy. In contrast, there may be sub-populations of stage II or IIIA patients who have such good prognosis that they may neither need nor derive benefit from adjuvant therapy.

Several groups have attempted to identify these sub-populations by studying the mRNA expression profiles of surgically excised tumor samples using high-density microarray platforms (11-17). Several groups, including our own, have reported smaller prognostic signatures assayed by quantitative reverse-transcriptase PCR (RT-PCR) (18). However the specific signatures identified by these groups show minimal overlap (19) and it is unclear why this is so. Ein-Dor and coworkers demonstrated that biological heterogeneity leads to thousands of samples being required to identify robust and reproducible subsets for most tumour types (20). These conclusions are supported by the finding that thousands of genes display intra-tumor heterogeneity, likely caused by the diversity of tumour microenvironments and cell populations (21, 22). We hypothesized that different statistical methods handle the disease heterogeneity in different ways, and thus play a major role in the lack of overlap amongst reported NSCLC prognostic signatures.

### SUMMARY OF THE INVENTION

In accordance with one aspect, there is provided a method for identifying a biomarker associated with a biological parameter comprising:
(a) providing a training dataset comprising the expression levels of a predetermined number (g) of genes from a cohort of subjects;
(b) selecting a set size (n);
(c) defining a plurality (S) of sets of genes, each set (s) having (n) genes uniquely selected from (g).
(d) for each (s), classifying subjects associated with that set into one of at least two populations (P) based on application of a partitioning method to the expression levels of such set, and repeating the foregoing for all sets of genes;
(e) providing one or more validation datasets, each comprising the expression levels of the predetermined number genes from one or more validation cohorts of subjects;
(f) for each (s) in each validation dataset, classifying subjects associated with that (s) into one of the at least two (P) based on the distance to the expression levels of (s) from the subjects in the training dataset, and repeating the foregoing for all sets of genes;
(g) determining the relationship between the biological parameter and each (P);
(h) rank sets based on strength of the relationship determined in step (g);
(i) select high strength sets having a strength greater than a predetermined set threshold;
(j) identify genes in the high strength sets that are enriched above a predetermined enrichment threshold.

In accordance with a further aspect, there is provided a computer readable memory having recorded thereon statements and instructions for execution by a computer to carry out the method described herein.

In accordance with a further aspect, there is provided a computer program product, comprising a memory having a computer readable code embodied therein, for execution by a CPU, said code comprising code means for each of the steps of the method described herein.

In accordance with a further aspect, there is provided a method for identifying a gene signature associated with a biological parameter comprising:
(a) providing a training dataset comprising molecular characteristics of genes (g) from a cohort of subjects;
(b) selecting a set size (n);
(c) defining a plurality (S) of set of genes, each set (s) having (n) genes uniquely selected from (g).
(d) for each (s), classifying subjects associated with that set into one of at least two populations (P) based on application of a partitioning method to the molecular characteristics of such set, and repeating the foregoing for all sets of genes;
(e) providing one or more validation datasets, each comprising molecular characteristics of the predetermined number genes from one or more validation cohorts of subjects;
(f) for each (s) in each validation dataset, classifying subjects associated with that (s) into one of the at least two (P) based on the distance to the expression levels of (s) from the subjects in the training dataset, and repeating the foregoing for all sets of genes;
(g) determination the relationship between the biological parameter and each (P);
(h) rank sets based on strength of the relationship determined in step (g);
(i) select high strength sets having a strength greater than a predetermined set threshold;
(j) identify genes in the high strength sets that are enriched above a predetermined enrichment threshold.

In accordance with a further aspect, there is provided a method of prognosing or classifying a subject with non-small cell lung cancer NSCLC comprising:
(a) determining the expression of at least three biomarkers in a test sample from the subject selected from CALCA, CCR7, STX1A, CCT3, SPRR1B, SELP, PAFAH1B3, CPE, XRCC6, HIF1A, MARCH6, PLOD2, NAP1L1, SFTPC, KRT5 and STC1; and
(b) comparing expression of the at least three biomarkers in the test sample with expression of the at least three biomarkers in a control sample;
wherein a difference or similarity in the expression of the at least three biomarkers between the control and the test sample is used to prognose or classify the subject with NSCLC into a poor survival group or a good survival group.

In accordance with a further aspect, there is provided a method of predicting prognosis in a subject with non-small cell lung cancer (NSCLC) comprising the steps:
(a) obtaining a subject biomarker expression profile in a sample of the subject;
(b) obtaining a biomarker reference expression profile associated with a prognosis, wherein the subject biomarker expression profile and the biomarker reference expression profile each have values representing the expression level of at least three biomarkers selected from CALCA, CCR7, STX1A, CCT3, SPRR1B, SELP, PAFAH1B3, CPE, XRCC6, HIF1A, MARCH6, PLOD2, NAP1L1, SFTPC, KRT5 and STC1;
(c) selecting the biomarker reference expression profile most similar to the subject biomarker expression profile, to thereby predict a prognosis for the subject.

In accordance with a further aspect, there is provided a method of selecting a therapy for a subject with NSCLC, comprising the steps:
(a) classifying the subject with NSCLC into a poor survival group or a good survival group according to the method of any one of claims 1-23; and
(b) selecting adjuvant chemotherapy for the poor survival group or no adjuvant chemotherapy for the good survival group.

In accordance with a further aspect, there is provided a method of selecting a therapy for a subject with NSCLC, comprising the steps:
(a) determining the expression of at least three biomarkers in a test sample from the subject selected from CALCA, CCR7, STX1A, CCT3, SPRR1B, SELP, PAFAH1B3, CPE, XRCC6, HIF1A, MARCH6, PLOD2, NAP1L1, SFTPC, KRT5 and STC1;
(b) comparing the expression of the at least three biomarkers in the test sample with the at least three biomarkers in a control sample;
(c) classifying the subject in a poor survival group or a good survival group, wherein a difference or a similarity in the expression of the at least three biomarkers between the control sample and the test sample is used to classify the subject into a poor survival group or a good survival group;
(d) selecting adjuvant chemotherapy if the subject is classified in the poor survival group and selecting no adjuvant chemotherapy if the subject is classified in the good survival group.

In accordance with a further aspect, there is provided a composition comprising a plurality of isolated nucleic acid sequences, wherein each isolated nucleic acid sequence hybridizes to:
(a) a RNA product of at least three of sixteen genes: CALCA, CCR7, STX1A, CCT3, SPRR1B, SELP, PAFAH1B3, CPE, XRCC6, HIF1A, MARCH6, PLOD2, NAP1L1, SFTPC, KRT5 and STC1; and/or
(b) a nucleic acid complementary to a),
wherein the composition is used to measure the level of RNA expression of the genes.

In accordance with a further aspect, there is provided an array comprising, for each of at least three of sixteen genes: CALCA, CCR7, STX1A, CCT3, SPRR1B, SELP, PAFAH1B3, CPE, XRCC6, HIF1A, MARCH6, PLOD2, NAP1L1, SFTPC, KRT5 and STC1, one or more polynucleotide probes complementary and hybridizable to an expression product of the gene.

In accordance with a further aspect, there is provided a computer program product for use in conjunction with a computer having a processor and a memory connected to the processor, the computer program product comprising a computer readable storage medium having a computer mechanism encoded thereon, wherein the computer program mechanism may be loaded into the memory of the computer and cause the computer to carry out the method described herein.

In accordance with a further aspect, there is provided a computer implemented product for predicting a prognosis or classifying a subject with NSCLC comprising:
(a) a means for receiving values corresponding to a subject expression profile in a subject sample; and
(b) a database comprising a reference expression profile associated with a prognosis, wherein the subject biomarker expression profile and the biomarker reference profile each have at least three values representing the expression level of at least three biomarkers selected from CALCA, CCR7, STX1A, CCT3, SPRR1B, SELP, PAFAH1B3, CPE, XRCC6, HIF1A, MARCH6, PLOD2, NAP1L1, SFTPC, KRT5 and STC1;
wherein the computer implemented product selects the biomarker reference expression profile most similar to the subject biomarker expression profile, to thereby predict a prognosis or classify the subject.

In accordance with a further aspect, there is provided a computer implemented product for determining therapy for a subject with NSCLC comprising:
(a) a means for receiving values corresponding to a subject expression profile in a subject sample; and
(b) a database comprising a reference expression profile associated with a therapy, wherein the subject biomarker expression profile and the biomarker reference profile each has at least three values, each value representing the expression level of at least three biomarkers selected from CALCA, CCR7, STX1A, CCT3, SPRR1B, SELP, PAFAH1B3, CPE, XRCC6, HIF1A, MARCH6, PLOD2, AP1L1, SFTPC, KRT5 and STC1;
wherein the computer implemented product selects the biomarker reference expression profile most similar to the subject biomarker expression profile, to thereby predict the therapy.

In accordance with a further aspect, there is provided a computer readable medium having stored thereon a data structure for storing the computer implemented product described herein.

In accordance with a further aspect, there is provided a computer system comprising
(a) a database including records comprising a biomarker reference expression profile of at least three genes selected from CALCA, CCR7, STX1A, CCT3, SPRR1B, SELP, PAFAH1B3, CPE, XRCC6, HIF1A, MARCH6, PLOD2, NAP1L1, SFTPC, KRT5 and STC1 associated with a prognosis or therapy;
(b) a user interface capable of receiving a selection of gene expression levels of the at least three genes for use in comparing to the biomarker reference expression profile in the database;
(c) an output that displays a prediction of prognosis or therapy according to the biomarker reference expression profile most similar to the expression levels of the at least three genes.

In accordance with a further aspect, there is provided a kit to prognose or classify a subject with early stage NSCLC, comprising detection agents that can detect the expression products of at least three biomarkers selected from CALCA, CCR7, STX1A, CCT3, SPRR1B, SELP, PAFAH1B3, CPE, XRCC6, HIF1A, MARCH6, PLOD2, NAP1L1, SFTPC, KRT5 and STC1, and instructions for use.

In accordance with a further aspect, there is provided a kit to select a therapy for a subject with NSCLC, comprising detection agents that can detect the expression products of at least three biomarkers selected from CALCA, CCR7, STX1A, CCT3, SPRR1B, SELP, PAFAH1B3, CPE, XRCC6, HIF1A, MARCH6, PLOD2, NAP1L1, SFTPC, KRT5 and STC1, and instructions for use.

### BRIEF DESCRIPTION OF THE DRAWINGS

These and other features of the preferred embodiments of the invention will become more apparent in the following detailed description in which reference is made to the appended drawings wherein:

FIG. 1 shows the modified steepest descent algorithm trained on a RT-PCR dataset of 158 genes in 147 NSCLC patients. The resulting six-gene classifier separated patients into two groups with significantly different outcomes (A). Leave-one-out cross-validation again identified two groups with significantly different outcomes (B). The number of patients at risk at each time-interval in the molecularly-defined good and poor prognosis groups is listed below each survival curve. The stage-adjusted hazard ratio (HR), p-value (Wald test), and number of patients classified (N) are given on each survival curve.

FIG. 2 shows classification of patients from four independent datasets. (A) Mixed adenocarcinomas and squamous cell carcinomas profiled with Affymetrix HG-U133Plus2 arrays by Potti et al. (15). (B) Adenocarcinomas profiled on cDNA arrays by Larsen et al. (13). (C) Squamous cell carcinomas profiled on Affymetrix HG-U133A arrays by Raponi et al. (16). (D) Squamous cell carcinomas profiled on cDNA arrays by Larsen et al. (14). The number of patients at risk in each molecularly-defined group is indicated at several time-points. The stage-adjusted hazard ratio (HR) and p-value (Wald test), and the number of patients successfully classified (N) are also shown.

FIG. 3 shows permutation validation of ten million six-gene signatures generated at random from our training dataset. A log-rank test was performed on each signature and the Gaussian kernel density of the chi-squared values from this log-rank test was generated (A). The x-axis indicates the chi-squared values: larger values indicate a lower p-value and hence a more statistically significant separation of patient groups. The y-axis gives the kernel density, which reflects the probability distribution of the dataset. Higher values indicate larger fraction of the population, akin to a smoothed histogram. The performance of the mSD signature is marked with an arrow. These ten million trained signatures were then tested in four independent datasets. Kernel density estimates, as above, are provided for each test dataset (B-E). Each test dataset is labeled with the name of the first author of the study. The performance of the mSD signature is marked with an arrow. Validation scores were generated by multiplying the percentile rankings of each signature in each of the four test datasets. Higher values thus correspond to improved validation across all four datasets. The performance of the mSD signature is marked with an arrow.

FIG. 4 shows the fraction of six gene signatures containing each gene that are statistically significant at p<0.05 (A). A zoom-in on the ten most enriched genes is also shown (B). The horizontal line represents the 5% level expected by chance alone, the y-axis gives the fraction of signatures containing that gene that are significant at p<0.05 and individual genes are on the x-axis.

FIG. 5 is a schematic showing the outline of the mSD procedure comprising two components: a prognosis-prediction component and a feature-selection component.

FIG. 6 shows clustering of the training dataset. Specifically, the expression profiles of the six-genes from the mSD-signature for the 147 patients of the training dataset were subjected to unsupervised pattern-recognition. Agglomerative hierarchical clustering using complete linkage was performed. The columns represent genes and the rows represent individual patients. The six genes all show unique expression patterns, as indicated by the long terminal arms of the column dendrogram. Patients do not fall into one or two large clusters, but rather into a diversity of small, non-linear ones, as indicated by the row dendrogram.

FIG. 7 shows classifier validation in a pooled dataset. Data from 8 studies was pooled into a dataset of 589 patients. The six-gene classifier separated all (A) and stage I patients (B) into groups with significantly different survival. The number of patients at risk in each molecularly-defined group is indicated at each time-point. The stage-adjusted hazard ratio (HR) and p-value (Wald test), and the number of patients successfully classified (N) are also shown.

FIG. 8 shows a summary of the validation datasets listed along the top of the chart, while various papers are listed along the side, identified by the first author. Each dataset is annotated according to which studies used it. Training datasets are marked with gray, while validation datasets are marked with solid black. The current study is highly validated, assessing eight distinct datasets. Some key clinical characteristics of each dataset are listed. AD=adenocarcinoma. SQ=squamous cell carcinoma.

### BRIEF DESCRIPTION OF THE TABLES

Table 1 shows univariate properties of the six-gene signature. Stable (Entrez Gene ID) identifiers and the independent univariate prognostic ability (based on the log-rank test and Cox proportional hazards modeling) are given for each component of the six-gene mSD signature.

Table 2 shows a summary of all patient data. The survival, follow-up status, clinical stage, and normalized expression levels for the six-gene signature of all patients considered in any analysis in this study. Patients are identified by the study of origin: UHN, Lau et al.; MI02, Beer et al.; MIT, Bhattacharjee et al.; Duke, Potti et al.; MI06, Raponi et al.; AD1, Larsen et al.; SQ2, Larsen et al.; LuMayo and LuWashU, Lu et al. mSD prediction status is also given for the training (UHN) dataset.

Table 3 shows a summary of mSD validation. For each validation dataset considered in this experiment, the number of patients, hazard ratio and 95% confidence interval, and p-value are given. The hazard ratio and p-value are derived from stage-adjusted Cox proportional hazard models, with p-values determined using the Wald test.

Table 4 shows a summary of permutation analyses for the training (UHN) and four validation (Duke, MI02, MI06, MIT) datasets. This table gives the total number of permutations considered, the number of missing values, the number and percentage of permutations statistically significant at p<0.05 (corresponding to chi-squared>3.84), the chi-squared value obtained from the mSD signature, and the number and percentage of permutations showing superior performance to the mSD signature. Missing values occur when clustering or classifying results in groups with such unequal sizes that log-rank analysis could not be performed. This occurred in approximately 0.01% of cases, and as such makes a negligible contribution to the overall classifier evaluation. Datasets are identified by the first author of the publication first reporting them.

Table 5 shows enrichment scores. Specifically, for each of the 113 genes in the permutation dataset the total number of signatures was counted containing that gene and the fraction of those signatures that are statistically significant at p<0.05 (chi-squared>3.84). Genes were then ranked by this enrichment score. The Gene ID gives the integer used to identify this gene in the raw permutation data. The official gene symbol uniquely identifies each gene in the dataset. The p-value for each gene is in the right-most column.

### DETAILED DESCRIPTION

The application generally relates to identifying gene signatures and provides methods and computer implemented products therefore.

The application also relates to 16 biomarkers that form a 16-gene signature, and provides methods, compositions, computer implemented products, detection agents and kits for prognosing or classifying a subject with non-small cell lung cancer (NSCLC) and for determining the benefit of adjuvant chemotherapy.

It must be noted that as used herein and in the appended claims, the singular forms “a”, “an” and “the” include the plural referents unless the context clearly dictates otherwise.

As used herein, “biological parameter” may refer to any measurable or quantifiable characteristic in a biological system and includes, without limitation, physical characteristics and attributes, genotype, phenotype, biomarkers, gene expression, splice-variants of an mRNA, polymorphisms of DNA or protein, levels of protein, cells, nucleic acids, amino acids or other biological matter.

The term “biomarker” as used herein refers to a gene that is differentially expressed in individuals. For example, specifically with respect to non-small cell lung cancer (NSCLC), the biomarkers may be differentially expressed in individuals according to prognosis and thus may be predictive of different survival outcomes and of the benefit of adjuvant chemotherapy. In one embodiment, the 16 biomarkers that form the NSCLC gene signature of the present application are listed as the first 16 genes in Table 5.

The term “level of expression” or “expression level” as used herein refers to a measurable level of expression of the products of biomarkers, such as, without limitation, the level of messenger RNA transcript expressed or of a specific exon or other portion of a transcript, the level of proteins or portions thereof expressed of the biomarkers, the number or presence of DNA polymorphisms of the biomarkers, the enzymatic or other activities of the biomarkers, and the level of specific metabolites.

The term “dataset” as used herein refers to the measurement or detection of one or more biological parameters for a series of subjects or individuals. Typically, a dataset will be generated at a single location or will involve measurements of biological parameters performed in a consistent manner. For example the set of expression levels of different mRNAs and survival times for one or more individuals with non-small cell-lung cancer would comprise a “dataset”.

The term “partitioning method” as used herein refers to a method that divides a dataset into two or more groups along any dimension of the dataset using either features inherent to the dataset or external meta-information. The number of groups can be as large as the dimension of the dataset or can be a continuous variable. For example k-means clustering, median-dichotomization, novelty-detection, and hierarchical clustering are all partitioning methods and others would be known to a person skilled in the art.

The term “strength” as used herein refers to the predictive power that a biomarker has for a specific biological parameter. Predictive power can be assessed by methods known to a person skilled in the art and include, without limitation, using measures of magnitude, such as differences in survival rates or hazard ratios, or using prediction accuracies or measures of statistical significance such as p-values. Methods also exist to consider both magnitude and statistical significance, such as the F-statistic.

The term “set threshold” as used herein refers to a threshold value of the strength of the relationship between a biomarker and a biological parameter that is used to identify biomarkers that have a meaningful association with a biological parameter. The specific value of the set threshold is dependent on the parameter used to measure the strength of the association. For example if hazard-ratios are used to measure the magnitude of a predictive threshold than a set threshold might be a hazard ratio greater than two. For example if p-values are used to measure the reproducibility of a biomarker then a set threshold might be a p-value less than 0.05. For example if prediction accuracies are used to measure the reproducibility of an association then a set threshold might be a prediction accuracy greater than 70%.

The term “enrichment threshold” as used herein refers to a threshold value of the number of sets in which a gene is found where that set has a strong association with a biological parameter as determined by the set threshold. For example, an enrichment threshold might be a fraction of sets containing a specific such as 20%. Thus in this example if at least 20% of sets containing a specific gene have a strong association with the biological parameter then this gene will be above the enrichment threshold. An enrichment threshold might also be a p-value derived from a chi-squared test, a hypergeometric distribution, a proportion-test, and a permutation-based estimate of the null distribution, amongst others.

The term “molecular characteristics” as used herein refers to measurements of properties of the molecular composition of a biological specimen including, but not limited to, measurements of the levels or structural variations of specific mRNA transcripts or portions thereof, measurements of the levels of specific non-coding RNA species or portions thereof, measurements of the levels or structural variations of specific proteins including post-translational modifications thereof, measurements of the activity of specific proteins or complexes containing proteins, measurements of the number or type of genetic or epigenetic polymorphisms, and measurements of the levels of specific organic or inorganic metabolites within a cell.

According to an aspect, there is provided method for identifying a biomarker associated with a biological parameter comprising:
(d) providing a training dataset comprising the expression levels of a predetermined number (g) of genes from a cohort of subjects;
(e) selecting a set size (n);
(f) defining a plurality (S) of sets of genes, each set (s) having (n) genes uniquely selected from (g).
(g) for each (s), classifying subjects associated with that set into one of at least two populations (P) based on application of a partitioning method to the expression levels of such set, and repeating the foregoing for all sets of genes;
(h) providing one or more validation datasets, each comprising the expression levels of the predetermined number genes from one or more validation cohorts of subjects;
(i) for each (s) in each validation dataset, classifying subjects associated with that (s) into one of the at least two (P) based on the distance to the expression levels of (s) from the subjects in the training dataset, and repeating the foregoing for all sets of genes;
(j) determining the relationship between the biological parameter and each (P);
(k) rank sets based on strength of the relationship determined in step (g);
(l) select high strength sets having a strength greater than a predetermined set threshold;
(m) identify genes in the high strength sets that are enriched above a predetermined enrichment threshold.

Preferably, there is at least two validation datasets and between steps (h) and (i), further comprising the step of pooling the ranks determined in step (h) for each validation dataset.

In one embodiment, the ranks are expressed as percentiles and the pooling comprises the product the percentiles.

Pooling may also be performed using other methods known by a person skilled in the art. For example, without limitation, pooling may be performed using a standard dataset and machine-learning methods such as support vector machines or random forests, or pooling may be performed by taking the product of the p-values of a statistical test of the strength of the association of a biomarker with a biological parameter, or pooling may be performed by taking the sum or product (weighted or unweighted) of the magnitudes of the strength of the association of a biomarker with a biological parameter. For example, the sum of hazard ratios or of coefficients from a Cox proportional hazard model across multiple validation datasets could be used to pool validation datasets.

In some embodiments, there is at least two validation datasets and after step (i), further comprising the step of determining those genes identified in (j) that were enriched above the predetermined enrichment threshold in a plurality of validation datasets.

In some embodiments, the partitioning method comprises k-means clustering. However, other partitioning methods would be known to a person skilled in the art, for example, without limitation, agglomerative hierarchical clustering, divisive hierarchical clustering, novelty-detection, median dichotomization, asymmetric thresholding and self-organizing maps. Preferably, this embodiment additionally comprises performing a log-rank analysis to estimate the separation between the at least two populations. However, a person skilled in the art would understand that other methods could be used, for example, without limitation, Cox proportional hazards modeling with or without adjustment for clinical parameters, Wilcoxon Rank-Sum analysis, t-test analysis, general linear modeling, and non-linear mixed modeling.

In some embodiments, the classifying in step (f) comprises calculation of Euclidian distance to determine the distance to the expression levels of s from the subjects in the training dataset. It is readily apparent to one skilled in the art that many alternative methods exist to determine the distance to the expression levels of s from the subjects in the training set, including but not limited to Pearson's correlation, k-nearest neighbours, classification in a hyperspace such as by support-vector machines, Manhattan distance, and mutual information.

In some embodiments, the relationship between the biological parameter and each (P) is determined using log-rank analysis. It is readily apparent to one skilled in the art that many alternative methods exist to determine this relationship, including but not limited to Cox proportional hazards modeling with or without adjustment for other clinical covariates, Wilcoxon rank-sum analysis, general linear modeling, and linear or non-linear mixed modeling.

In some embodiments, the set size n is between 2 and 20, preferably between 4 and 18, 4 and 14, 4 and 10, and 6 and 8 in increasing preferablity.

In some embodiments, the number of genes (m) is between 3 and 10,000, preferably between 20 and 200.

In some embodiments, the plurality (S) of sets of genes is the smaller of 1,000,000 and 0.1% of all possible sets of m genes having n set size.

In some embodiments, the validation dataset at least partially overlaps with the training dataset.

In accordance with a further aspect, there is provided a computer readable memory having recorded thereon statements and instructions for execution by a computer to carry out the method described herein.

In accordance with a further aspect, there is provided a computer program product, comprising a memory having a computer readable code embodied therein, for execution by a CPU, said code comprising code means for each of the steps of the method described herein.

In accordance with a further aspect, there is provided a method for identifying a gene signature associated with a biological parameter comprising:
(a) providing a training dataset comprising molecular characteristics of genes (g) from a cohort of subjects;
(b) selecting a set size (n);
(c) defining a plurality (S) of set of genes, each set (s) having n genes uniquely selected from (g).
(d) for each (s), classifying subjects associated with that set into one of at least two populations (P) based on application of a partitioning method to the molecular characteristics of such set, and repeating the foregoing for all sets of genes;
(e) providing one or more validation datasets, each comprising molecular characteristics of the predetermined number genes from one or more validation cohorts of subjects;
(f) for each (s) in each validation dataset, classifying subjects associated with that (s) into one of the at least two (P) based on the distance to the expression levels of (s) from the subjects in the training dataset, and repeating the foregoing for all sets of genes;
(g) determination the relationship between the biological parameter and each (P);
(h) rank sets based on strength of the relationship determined in step (g);
(i) select high strength sets having a strength greater than a predetermined set threshold;

(j) identify genes in the high strength sets that are enriched above a predetermined enrichment threshold.

In accordance with a further aspect, there is provided a method of prognosing or classifying a subject with non-small cell lung cancer NSCLC comprising:
(k) determining the expression of at least three biomarkers in a test sample from the subject selected from CALCA, CCR7, STX1A, CCT3, SPRR1B, SELP, PAFAH1B3, CPE, XRCC6, HIF1A, MARCH6, PLOD2, NAP1L1, SFTPC, KRT5 and STC1; and
(l) comparing expression of the at least three biomarkers in the test sample with expression of the at least three biomarkers in a control sample;
wherein a difference or similarity in the expression of the at least three biomarkers between the control and the test sample is used to prognose or classify the subject with NSCLC into a poor survival group or a good survival group.

In accordance with a further aspect, there is provided a method of predicting prognosis in a subject with non-small cell lung cancer (NSCLC) comprising the steps:
(m) obtaining a subject biomarker expression profile in a sample of the subject;
(n) obtaining a biomarker reference expression profile associated with a prognosis, wherein the subject biomarker expression profile and the biomarker reference expression profile each have values representing the expression level of at least three biomarkers selected from CALCA, CCR7, STX1A, CCT3, SPRR1B, SELP, PAFAH1B3, CPE, XRCC6, HIF1A, MARCH6, PLOD2, NAP1L1, SFTPC, KRT5 and STC1;
(o) selecting the biomarker reference expression profile most similar to the subject biomarker expression profile, to thereby predict a prognosis for the subject.

Preferably, the biomarker reference expression profile comprises a poor survival group or a good survival group.

The term “reference expression profile” as used herein refers to the expression level of at least 3 of the 16 biomarkers selected from CALCA, CCR7, STX1A, CCT3, SPRR1B, SELP, PAFAH1B3, CPE, XRCC6, HIF1A, MARCH6, PLOD2, NAP1L1, SFTPC, KRT5 and STC1 associated with a clinical outcome in a NSCLC patient. The reference expression profile comprises 16 values, each value representing the level of a biomarker, wherein each biomarker corresponds to one gene selected from CALCA, CCR7, STX1A, CCT3, SPRR1B, SELP, PAFAH1B3, CPE, XRCC6, MARCH6, PLOD2, NAP1L1, SFTPC, KRT5 and STC1. The reference expression profile is identified using one or more samples comprising tumor or adjacent or otherwise tumour-related stromal/blood based tissue or cells, wherein the expression is similar between related samples defining an outcome class or group such as poor survival or good survival and is different to unrelated samples defining a different outcome class such that the reference expression profile is associated with a particular clinical outcome. The reference expression profile is accordingly a reference profile or reference signature of the expression of at least 3 of the 16 biomarkers selected from CALCA, CCR7, STX1A, CCT3, SPRR1B, SELP, PAFAH1B3, CPE, XRCC6, HIF1A, MARCH6, PLOD2, NAP1L1, SFTPC, KRT5 and STC1, to which the subject expression levels of the corresponding genes in a patient sample are compared in methods for determining or predicting clinical outcome.

As used herein, the term “control” refers to a specific value or dataset that can be used to prognose or classify the value e.g expression level or reference expression profile obtained from the test sample associated with an outcome class. In one embodiment, a dataset may be obtained from samples from a group of subjects known to have NSCLC and good survival outcome or known to have NSCLC and have poor survival outcome or known to have NSCLC and have benefited from adjuvant chemotherapy or known to have NSCLC and not have benefited from adjuvant chemotherapy. The expression data of the biomarkers in the dataset can be used to create a control value that is used in testing samples from new patients. In such an embodiment, the “control” is a predetermined value for the set of at least 3 of the 16 biomarkers obtained from NSCLC patients whose biomarker expression values and survival times are known. Alternatively, the “control” is a predetermined reference profile for the set of at least three of the sixteen biomarkers described herein obtained from patients whose survival times are known.

Accordingly, in one embodiment, the control is a sample from a subject known to have NSCLC and good survival outcome. In another embodiment, the control is a sample from a subject known to have NSCLC and poor survival outcome.

A person skilled in the art will appreciate that the comparison between the expression of the biomarkers in the test sample and the expression of the biomarkers in the control will depend on the control used. For example, if the control is from a subject known to have NSCLC and poor survival, and there is a difference in expression of the biomarkers between the control and test sample, then the subject can be prognosed or classified in a good survival group. If the control is from a subject known to have NSCLC and good survival, and there is a difference in expression of the biomarkers between the control and test sample, then the subject can be prognosed or classified in a poor survival group. For example, if the control is from a subject known to have NSCLC and good survival, and there is a similarity in expression of the biomarkers between the control and test sample, then the subject can be prognosed or classified in a good survival group. For example, if the control is from a subject known to have NSCLC and poor survival, and there is a similarity in expression of the biomarkers between the control and test sample, then the subject can be prognosed or classified in a poor survival group.

A person skilled in the art will appreciate that the comparison between the expression of the biomarkers in the test sample and the expression of the biomarkers in the control can be made in different ways. For example, without limitation, Euclidean distances, Pearson's correlation, and k-nearest neighbours can be used to determine the similarity of the expression of the biomarkers in the test sample to the expression of the biomarkers in the control sample.

The term “differentially expressed” or “differential expression” as used herein refers to a difference in the level of expression of the biomarkers that can be assayed by measuring the level of expression of the products of the biomarkers, such as the difference in level of messenger RNA transcript or a portion thereof expressed or of proteins expressed of the biomarkers. In a preferred embodiment, the difference is statistically significant. The term “difference in the level of expression” refers to an increase or decrease in the measurable expression level of a given biomarker, for example as measured by the amount of messenger RNA transcript and/or the amount of protein in a sample as compared with the measurable expression level of a given biomarker in a control. In one embodiment, the differential expression can be compared using the ratio of the level of expression of a given biomarker or biomarkers as compared with the expression level of the given biomarker or biomarkers of a control, wherein the ratio is not equal to 1.0. For example, an RNA or protein is differentially expressed if the ratio of the level of expression in a first sample as compared with a second sample is greater than or less than 1.0. For example, a ratio of greater than 1, 1.2, 1.5, 1.7, 2, 3, 3, 5, 10, 15, 20 or more, or a ratio less than 1, 0.8, 0.6, 0.4, 0.2, 0.1, 0.05, 0.001 or less. In another embodiment the differential expression is measured using p-value. For instance, when using p-value, a biomarker is identified as being differentially expressed as between a first sample and a second sample when the p-value is less than 0.1, preferably less than 0.05, more preferably less than 0.01, even more preferably less than 0.005, the most preferably less than 0.001.

The term “similarity in expression” as used herein means that there is no or little difference in the level of expression of the biomarkers between the test sample and the control or reference profile. For example, similarity can refer to a fold difference compared to a control. In a preferred embodiment, there is no statistically significant difference in the level of expression of the biomarkers.

The term “most similar” in the context of a reference profile refers to a reference profile that is associated with a clinical outcome that shows the greatest number of identities and/or degree of changes with the subject profile.

The term “prognosis” as used herein refers to a clinical outcome group such as a poor survival group or a good survival group associated with a disease subtype which is reflected by a reference profile such as a biomarker reference expression profile or reflected by an expression level of the fifteen biomarkers disclosed herein. The prognosis provides an indication of disease progression and includes an indication of likelihood of death due to lung cancer. In one embodiment the clinical outcome class includes a good survival group and a poor survival group.

The term “prognosing or classifying” as used herein means predicting or identifying the clinical outcome group that a subject belongs to according to the subject's similarity to a reference profile or biomarker expression level associated with the prognosis. For example, prognosing or classifying comprises a method or process of determining whether an individual with NSCLC has a good or poor survival outcome, or grouping an individual with NSCLC into a good survival group or a poor survival group, or predicting whether or not an individual with NSCLC will respond to therapy.

The term “good survival” as used herein refers to an increased chance of survival as compared to patients in the “poor survival” group. For example, the biomarkers of the application can prognose or classify patients into a “good survival group”. These patients are at a lower risk of death after surgery.

The term “poor survival” as used herein refers to an increased risk of death as compared to patients in the “good survival” group. For example, biomarkers or genes of the application can prognose or classify patients into a “poor survival group”. These patients are at greater risk of death or adverse reaction from disease or surgery, treatment for the disease or other causes.

Accordingly, in one embodiment, the biomarker reference expression profile comprises a poor survival group. In another embodiment, the biomarker reference expression profile comprises a good survival group.

The term “subject” as used herein refers to any member of the animal kingdom, preferably a human being and most preferably a human being that has NSCLC or that is suspected of having NSCLC.

In various embodiments, the at least three biomarkers is four, five, six, seven, eight, nine, ten, eleven, twelve, thirteen, fourteen, fifteen and sixteen biomarkers respectively.

In some embodiments the NSCLC is stage I or stage II.

NSCLC patients are classified into stages, which are used to determine therapy. Staging classification testing may include any or all of history, physical examination, routine laboratory evaluations, chest x-rays, and chest computed tomography scans or positron emission tomography scans with infusion of contrast materials. For example, stage I includes cancer in the lung, but has not spread to adjacent lymph nodes or outside the chest. Stage I is divided into two categories based primarily on the size of the tumor (IA and IB). Stage II includes cancer located in the lung and proximal lymph nodes. Stage II is divided into 2 categories based on the size of tumor and nodal status (IIA and IIB). Stage III includes cancer located in the lung and the lymph nodes. Stage III is divided into 2 categories based on the size of tumor and nodal status (IIIA and IIIB). Stage 1V includes cancer that has metastasized to distant locations. The term “early stage NSCLC” includes patients with Stage I to IIIA NSCLC. These patients are treated primarily by complete surgical resection.

The term “test sample” as used herein refers to any fluid, cell or tissue sample from a subject which can be assayed for biomarker expression products and/or a reference expression profile, e.g. genes differentially expressed in subjects with NSCLC according to survival outcome.

The phrase “determining the expression of biomarkers” as used herein refers to determining or quantifying RNA or proteins or protein activities or protein-related metabolites expressed by the biomarkers. The term “RNA” includes mRNA transcripts, and/or specific spliced or other alternative variants of mRNA, including anti-sense products. The term “RNA product of the biomarker” as used herein refers to RNA transcripts transcribed from the biomarkers and/or specific spliced or alternative variants. In the case of “protein”, it refers to proteins translated from the RNA transcripts transcribed from the biomarkers. The term “protein product of the biomarker” refers to proteins translated from RNA products of the biomarkers.

A person skilled in the art will appreciate that a number of methods can be used to detect or quantify the level of RNA products of the biomarkers within a sample, including arrays, such as microarrays, RT-PCR (including quantitative RT-PCR), nuclease protection assays and Northern blot analyses.

Accordingly, in one embodiment, the biomarker expression levels are determined using arrays, optionally microarrays, RT-PCR, optionally quantitative RT-PCR, nuclease protection assays or Northern blot analyses.

In another embodiment, the biomarker expression levels are determined by using an array. In one embodiment, the array is a HG-U133A chip from Affymetrix. In another embodiment, a plurality of nucleic acid probes that are complementary or hybridizable to an expression product of at least 3 of the 16 biomarkers selected from CALCA, CCR7, STX1A, CCT3, SPRR1B, SELP, PAFAH1B3, CPE, XRCC6, HIF1A, MARCH6, PLOD2, NAP1L1, SFTPC, KRT5 and STC1 are used on the array.

The term “nucleic acid” includes DNA and RNA and can be either double stranded or single stranded.

The term “hybridize” or “hybridizable” refers to the sequence specific non-covalent binding interaction with a complementary nucleic acid. In a preferred embodiment, the hybridization is under high stringency conditions. Appropriate stringency conditions which promote hybridization are known to those skilled in the art, or can be found in Current Protocols in Molecular Biology, John Wiley & Sons, N.Y. (1989), 6.3.1 6.3.6. For example, 6.0× sodium chloride/sodium citrate (SSC) at about 45° C., followed by a wash of 2.0×SSC at 50° C. may be employed.

The term “probe” as used herein refers to a nucleic acid sequence that will hybridize to a nucleic acid target sequence. In one example, the probe hybridizes to an RNA product of the biomarker or a nucleic acid sequence complementary thereof. The length of probe depends on the hybridization conditions and the sequences of the probe and nucleic acid target sequence. In one embodiment, the probe is at least 8, 10, 15, 20, 25, 50, 75, 100, 150, 200, 250, 400, 500 or more nucleotides in length.

In another embodiment, the biomarker expression levels are determined by using quantitative RT-PCR. In another embodiment, the primers used for quantitative RT-PCR comprise a forward and reverse primer for each of CALCA, CCR7, STX1A, CCT3, SPRR1B, SELP, PAFAH1B3, CPE, XRCC6, HIF1A, MARCH6, PLOD2, NAP1L1, SFTPC, KRT5 and STC1.

The term “primer” as used herein refers to a nucleic acid sequence, whether occurring naturally as in a purified restriction digest or produced synthetically, which is capable of acting as a point of synthesis when placed under conditions in which synthesis of a primer extension product, which is complementary to a nucleic acid strand is induced (e.g. in the presence of nucleotides and an inducing agent such as DNA polymerase and at a suitable temperature and pH). The primer must be sufficiently long to prime the synthesis of the desired extension product in the presence of the inducing agent. The exact length of the primer will depend upon factors, including temperature, sequences of the primer and the methods used. A primer typically contains 15-25 or more nucleotides, although it can contain less or more. The factors involved in determining the appropriate length of primer are readily known to one of ordinary skill in the art.

In addition, a person skilled in the art will appreciate that a number of methods can be used to determine the amount of a protein product of the biomarker of the invention, including immunoassays such as Western blots, ELISA, and immunoprecipitation followed by SDS-PAGE and immunocytochemistry.

Accordingly, in another embodiment, an antibody is used to detect the polypeptide products of at least 3 of the 16 biomarkers selected from CALCA, CCR7, STX1A, CCT3, SPRR1B, SELP, PAFAH1B3, CPE, XRCC6, HIF1A, MARCH6, PLOD2, NAP1L1, SFTPC, KRT5 and STC1. In another embodiment, the sample comprises a tissue sample. In a further embodiment, the tissue sample is suitable for immunohistochemistry.

The term “antibody” as used herein is intended to include monoclonal antibodies, polyclonal antibodies, and chimeric antibodies. The antibody may be from recombinant sources and/or produced in transgenic animals. The term “antibody fragment” as used herein is intended to include Fab, Fab′, F(ab′)2, scFv, dsFv, ds-scFv, dimers, minibodies, diabodies, and multimers thereof and bispecific antibody fragments. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques.

Conventional techniques of molecular biology, microbiology and recombinant DNA techniques are within the skill of the art. Such techniques are explained fully in the literature. See, e.g., Sambrook, Fritsch & Maniatis, 1989, Molecular Cloning: A Laboratory Manual, Second Edition; Oligonucleotide Synthesis (M. J. Gait, ed., 1984); Nucleic Acid Hybridization (B. D. Harms & S. J. Higgins, eds., 1984); A Practical Guide to Molecular Cloning (B. Perbal, 1984); and a series, Methods in Enzymology (Academic Press, Inc.); Short Protocols In Molecular Biology, (Ausubel et al., ed., 1995).

For example, antibodies having specificity for a specific protein, such as the protein product of a biomarker, may be prepared by conventional methods. A mammal, (e.g. a mouse, hamster, or rabbit) can be immunized with an immunogenic form of the peptide which elicits an antibody response in the mammal. Techniques for conferring immunogenicity on a peptide include conjugation to carriers or other techniques well known in the art. For example, the peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art, (e.g. the hybridoma technique originally developed by Kohler and Milstein (Nature 256:495-497 (1975)) as well as other techniques such as the human B-cell hybridoma technique (Kozbor et al., Immunol. Today 4:72 (1983)), the EBV-hybridoma technique to produce human monoclonal antibodies (Cole et al., Methods Enzymol, 121:140-67 (1986)), and screening of combinatorial antibody libraries (Huse et al., Science 246:1275 (1989)). Hybridoma cells can be screened immunochemically for production of antibodies specifically reactive with the peptide and the monoclonal antibodies can be isolated.

The gene signature described herein can be used to select treatment for NCSLC patients. As explained herein, the biomarkers can classify patients with NSCLC into a poor survival group or a good survival group and into groups that might benefit from adjuvant chemotherapy or not.

Accordingly, in one embodiment, the application provides a method of selecting a therapy for a subject with NSCLC, comprising the steps:
(a) classifying the subject with NSCLC into a poor survival group or a good survival group according to the methods described herein; and
(b) selecting adjuvant chemotherapy for the subject classified as being in the poor survival group or no adjuvant chemotherapy for the subject classified as being in the good survival group.

In another embodiment, the application provides a method of selecting a therapy for a subject with NSCLC, comprising the steps:
(a) determining the expression of at least 3 of the 16 biomarkers selected from CALCA, CCR7, STX1A, CCT3, SPRR1B, SELP, PAFAH1B3, CPE, XRCC6, HIF1A, MARCH6, PLOD2, NAP1L1, SFTPC, KRT5 and STC1 in a test sample from the subject;
(b) comparing the expression of the at least 3 of the 16 biomarkers in the test sample with the at least 4 of the 16 biomarkers in a control sample;
(c) classifying the subject into a poor survival group or a good survival group, wherein a difference or a similarity in the expression of the at least 3 of the 16 biomarkers between the control sample and the test sample is used to classify the subject into a poor survival group or a good survival group; and
(d) selecting adjuvant chemotherapy if the subject is classified in the poor survival group and selecting no adjuvant chemotherapy if the subject is classified in the good survival group.

The term “adjuvant chemotherapy” as used herein means treatment of cancer with chemotherapeutic agents after surgery where all detectable disease has been removed, but where there still remains a risk of small amounts of remaining cancer. Typical chemotherapeutic agents include cisplatin, carboplatin, vinorelbine, gemcitabine, doccetaxel, paclitaxel and navelbine.

In another aspect, the application provides compositions useful in detecting changes in the expression levels of at least 3 of the 16 biomarkers selected from CALCA, CCR7, STX1A, CCT3, SPRR1B, SELP, PAFAH1B3, CPE, XRCC6, HIF1A, MARCH6, PLOD2, NAP1L1, SFTPC, KRT5 and STC1. Accordingly in one embodiment, the application provides a composition comprising a plurality of isolated nucleic acid sequences wherein each isolated nucleic acid sequence hybridizes to:
(a) a RNA product of one of CALCA, CCR7, STX1A, CCT3, SPRR1B, SELP, PAFAH1B3, CPE, XRCC6, HIF1A, MARCH6, PLOD2, NAP1L1, SFTPC, KRT5 and STC1; and/or
(b) a nucleic acid complementary to a),
wherein the composition is used to measure the level of RNA expression of the 16 genes.

In a further aspect, the application also provides an array that is useful in detecting the expression levels of at least 3 of the 16 biomarkers selected from CALCA, CCR7, STX1A, CCT3, SPRR1B, SELP, PAFAH1B3, CPE, XRCC6, HIF1A, MARCH6, PLOD2, NAP1L1, SFTPC, KRT5 and STC1. Accordingly, in one embodiment, the application provides an array comprising for each of the above biomarkers one or more nucleic acid probes complementary and hybridizable to an expression product of the gene.

In yet another aspect, the application also provides for kits used to prognose or classify a subject with NSCLC into a good survival group or a poor survival group or to select a therapy for a subject with NSCLC that includes detection agents that can detect the expression products of the biomarkers. Accordingly, in one embodiment, the application provides a kit to prognose or classify a subject with early stage NSCLC comprising detection agents that can detect the expression products of at least 3 of the 16 biomarkers selected from CALCA, CCR7, STX1A, CCT3, SPRR1B, SELP, PAFAH1B3, CPE, XRCC6, HIF1A, MARCH6, PLOD2, NAP1L1, SFTPC, KRT5 and STC1. In another embodiment, the application provides a kit to select a therapy for a subject with NSCLC, comprising detection agents that can detect the expression products of at least 4 of the 16 biomarkers selected from CALCA, CCR7, STX1A, CCT3, SPRR1B, SELP, PAFAH1B3, CPE, XRCC6, HIF1A, MARCH6, PLOD2, NAP1L1, SFTPC, KRT5 and STC1.

A person skilled in the art will appreciate that a number of detection agents can be used to determine the expression of the biomarkers. For example, to detect RNA products of the biomarkers, probes, primers, complementary nucleotide sequences or nucleotide sequences that hybridize to the RNA products can be used. To detect protein products of the biomarkers, ligands or antibodies that specifically bind to the protein products can be used.

Accordingly, in one embodiment, the detection agents are probes that hybridize to the at least 4 of the sixteen biomarkers. A person skilled in the art will appreciate that the detection agents can be labeled.

The label is preferably capable of producing, either directly or indirectly, a detectable signal. For example, the label may be radio-opaque or a radioisotope, such as ³H, ¹⁴C, ³²P, ³⁵S, ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion.

The kit can also include a control or reference standard and/or instructions for use thereof. In addition, the kit can include ancillary agents such as vessels for storing or transporting the detection agents and/or buffers or stabilizers.

In a further aspect, the application provides computer programs and computer implemented products for carrying out the methods described herein. Accordingly, in one embodiment, the application provides a computer program product for use in conjunction with a computer having a processor and a memory connected to the processor, the computer program product comprising a computer readable storage medium having a computer mechanism encoded thereon, wherein the computer program mechanism may be loaded into the memory of the computer and cause the computer to carry out the methods described herein.

In another embodiment, the application provides a computer implemented product for predicting a prognosis or classifying a subject with NSCLC comprising:
(a) a means for receiving values corresponding to a subject expression profile in a subject sample; and
(b) a database comprising a reference expression profile associated with a prognosis, wherein the subject biomarker expression profile and the biomarker reference profile each has at least three values, each value representing the expression level of a biomarker, wherein each biomarker corresponds to one of CALCA, CCR7, STX1A, CCT3, SPRR1B, SELP, PAFAH1B3, CPE, XRCC6, HIF1A, MARCH6, PLOD2, NAP1L1, SFTPC, KRT5 and STC1;
   wherein the computer implemented product selects the biomarker reference expression profile most similar to the subject biomarker expression profile, to thereby predict a prognosis or classify the subject.

In yet another embodiment, the application provides a computer implemented product for determining therapy for a subject with NSCLC comprising:
(a) a means for receiving values corresponding to a subject expression profile in a subject sample; and
(b) a database comprising a reference expression profile associated with a therapy, wherein the subject biomarker expression profile and the biomarker reference profile each has at least 3 values, each value representing the expression level of a biomarker, wherein each biomarker corresponds to one of CALCA, CCR7, STX1A, CCT3, SPRR1B, SELP, PAFAH1B3, CPE, XRCC6, HIF1A, MARCH6, PLOD2, NAP1L1, SFTPC, KRT5 and STC1;
   wherein the computer implemented product selects the biomarker reference expression profile most similar to the subject biomarker expression profile, to thereby predict the therapy.

Another aspect relates to computer readable mediums such as CD-ROMs. In one embodiment, the application provides computer readable medium having stored thereon a data structure for storing a computer implemented product described herein.

In one embodiment, the data structure is capable of configuring a computer to respond to queries based on records belonging to the data structure, each of the records comprising:
(a) a value that identifies a biomarker reference expression profile of at least 3 of the 16 biomarkers selected from CALCA, CCR7, STX1A, CCT3, SPRR1B, SELP, PAFAH1B3, CPE, XRCC6, HIF1A, MARCH6, PLOD2, NAP1L1, SFTPC, KRT5 and STC1;
(b) a value that identifies the probability of a prognosis associated with the biomarker reference expression profile.

In another aspect, the application provides a computer system comprising
(a) a database including records comprising a biomarker reference expression profile of at least 3 of the 16 biomarkers selected from CALCA, CCR7, STX1A, CCT3, SPRR1B, SELP, PAFAH1B3, CPE, XRCC6, HIF1A, MARCH6, PLOD2, NAP1L1, SFTPC, KRT5 and STC1associated with a prognosis or therapy;
(b) a user interface capable of receiving a selection of gene expression levels of at least 3 of the 16 biomarkers selected from CALCA, CCR7, STX1A, CCT3, SPRR1B, SELP, PAFAH1B3, CPE, XRCC6, HIF1A, MARCH6, PLOD2, NAP1L1, SFTPC, KRT5 and STC1 for use in comparing to the biomarker reference expression profile in the database; and
(c) an output that displays a prediction of prognosis or therapy according to the biomarker reference expression profile most similar to the expression levels of the fifteen genes.

The advantages of the present invention are further illustrated by the following example. The example and its particular details set forth herein are presented for illustration only and should not be construed as a limitation on the claims of the present invention.

### Example

### Materials & Methods

### Prognostic Signature Identification by Modified Steepest Descent

To identify a subset of genes whose mRNA expression profile is predictive of patient prognosis we combined feature selection by greedy forward-selection with unsupervised pattern-recognition. We call this algorithm modified Steepest Descent, or “mSD”, this iterative algorithm adds genes to an existing classifier based on their ability to maximize the significance of a log-rank test on patient groups identified by k-medians clustering and will be described in further detail below.

To identify a signature comprising genes that are not ranked by some univariate criterion, we developed a discrete, greedy gradient-descent algorithm (i.e the mSD). mSD begins by considering all possible classifiers (signatures) of one dimension (gene), and selecting the best gene. Once this optimal single-gene classifier is identified, the algorithm proceeds to add additional dimensions (genes) sequentially, testing all possible subsets of two genes that contain the optimal single-gene classifier. This corresponds to testing all supersets of the single-gene classifier and taking the largest discrete step to improve classifier performance. This procedure iterates through higher dimensions, evaluating successive supersets of the best n-gene classifier identified thus far. The algorithm terminates when an n gene classifier is discovered whose performance is not exceeded by any n+1 gene superset of itself. At each stage of the feature selection, classifier performance is evaluated by using k-medians clustering with k=2 to separate patients into two groups. Note that clustering is used here as an exploratory technique, not as a significance-testing method (30,31). Next, survival differences between these two groups are assessed using the log-rank test. Gene selection was made on the basis of the chi-squared statistic from the log-rank test, and thus the termination criterion corresponds to finding an n gene classifier whose chi-squared score cannot be exceeded by adding any single additional gene. The final output of the algorithm is a subset of prognostic genes, along with a separation of patients into a group with good survival (the “good prognosis group”) and a group with poor survival (the “poor prognosis group”). A Cox proportional hazards model including stage was then fit to these group assignments. Hazard ratios for the classification were extracted, along with p-values based on the Wald test. Feature selection was implemented in Perl (v5.8.7) and was run on AIX (v5.2.0.0) on an IBM p690. Clustering employed the Algorithm::Cluster (v1.31) C library (32) via its Perl bindings. Survival analysis used the survival package (v2.20) in R (v2.0.1).

### Training Dataset

A previously published RT-PCR dataset of 158 genes assessed in 147 NSCLC patients (19) was used for training. Data were normalized as described previously (28). Training used the original clinical annotation; subsequent survival analyses were performed using updated annotations, which increased patient follow-up by an average of 5.2 months (Table 2).

Two genes (STX1A and HIF1A) from this signature overlap with our previously reported linear risk-score analysis (33). Because we employed the same training dataset for both algorithms we are able to investigate the effect this overlap has on patient classifications. We compared the patient-by-patient predictions of our earlier risk-score-derived three-gene signature and our current six-gene signature (Table 2). The three-gene signature did not classify 10 patients from the initial cohort of 147, leaving 137 patients classified by both methods. Of these, 108 (79%) were classified identically by both methods. Most of the 29 mismatches (24/29=83%) were classified as poor prognosis by the three-gene signature and good prognosis by the six-gene signature. Similar proportions of adenocarcinomas and squamous cell carcinomas were divergently classified (22.6% vs. 20.2%, p=0.904). The two classifiers showed somewhat greater divergence for stage I than stage II or III patients, although this was not statistically significant (25.6% vs. 13.7%, p=0.154). The few divergences observed reflect the use of median dichotomization in the risk-score analysis. Median dichotomization is a common statistical procedure used when the training groups cannot be defined a priori, and forces the good and poor prognosis groups to be equally sized in the training dataset. By contrast the semi-supervised approach used by the mSD algorithm finds groups that reflect the strongest trend within the training dataset, regardless of group sizes. This is done by using unsupervised pattern-recognition (clustering). As a result mSD identifies groups of unequal size (92 good and 55 poor prognosis patients) while the risk-score analysis identified groups of equal size (68 good and 69 poor prognosis patients). Despite this underlying algorithmic difference, these data show that the two classifiers concur on the classifications for the majority of patients and that the few divergent classifications are not strongly biased according to any clinical covariates.

### Cross Validation

To estimate the generalization error of the mSD method we performed leave-one-out cross validation (29). Each of the 147 patients was classified using clusters defined with the remaining 146 patients. Euclidean distances were used to classify patients and significance was assessed with a stage-adjusted Cox proportional hazards model.

Specifically, using the normalized dataset, each of the 147 patients was sequentially removed from the sample. The mSD algorithm was then trained on the remaining 146 patient samples to select a prognostic subset of genes, as outlined above. The Euclidean distance between the expression profile of the omitted patient and the median expression profiles of the good and poor prognosis groups of patients were then calculated. The patient was classified into the nearer of these two groups, and the entire procedure was repeated 147 times so that each patient was omitted once. A survival curve of the resulting classifications was then plotted, and a stage-adjusted Cox proportional hazards model fitted as above. Cross validation was performed in R (v2.4.1) using the survival package (v2.31).

### Independent Validation Datasets

Four independent public datasets were used for validation (13, 14, 16, 25). The normalized data were downloaded and a unique probe for each of the six genes in the six gene signature (see above regarding Training Dataset and Table 2) was identified in each dataset. Median scaling and house-keeping gene normalization (to the geometric mean of ACTB, BAT1, B2M, and TBP levels) were performed (28). Euclidean distances to the training clusters were used to classify each patient. Survival differences were assessed using stage-adjusted Cox proportional hazards models.

Specifically, the four independent, publicly available datasets were used to validate the six-gene classifier identified by modified steepest-descent (34-37). These datasets were not used to select the 158 genes in our study and thus each constitutes an independent validation dataset. Two validation datasets were generated using Affymetrix microarrays (36, 37) and two using custom cDNA arrays (34, 35). Two are comprised primarily of adenocarcinomas (34, 36) and two exclusively of squamous cell carcinomas (35, 37). In each case, the normalized data were downloaded from the GEO repository. ProbeSets or spots representing the genes involved in the signature were identified using NetAffx annotation for Affymetrix arrays (36, 37) and BLAST analysis against UniGene build Hs.199 (34, 35) for cDNA arrays. When multiple ProbeSets for a single gene were present, the Pearson's correlation between their vectors was calculated. If they were strongly correlated (R>0.75) they were collapsed by averaging; otherwise bl2seq analysis against the RefSeq mRNA for the gene in question was used to identify the best match. Median scaling was performed as described previously (38). House-keeping gene normalization was used for the two Affymetrix array platforms, as described above for the PCR analysis. Because only one of the four house-keeping genes used was available on the custom cDNA platforms so this normalization step was omitted.

For each validation dataset, the distance between the expression profile for each patient and the cluster centers (medians) identified from the training dataset were calculated. A patient was classified into the nearer cluster if the ratio of the distances between the profile and the two clusters was at least 0.9. This quality criterion was not used for the two studies with small sample sizes where one signature gene was not present on the array platform (34, 35). The resulting classifications were then tested to determine if our prognostic signature resulted in significant survival differences using Cox proportional hazards model with adjustment for stage in R (v2.4.1) using the survival library (v2.33) as previously described.

### Pooled Analysis

We combined patients from the four validation datasets described above with four older or smaller NSCLC datasets (11, 12, 23). These 589 patients were classified as described above, with Cox modeling to identify survival differences.

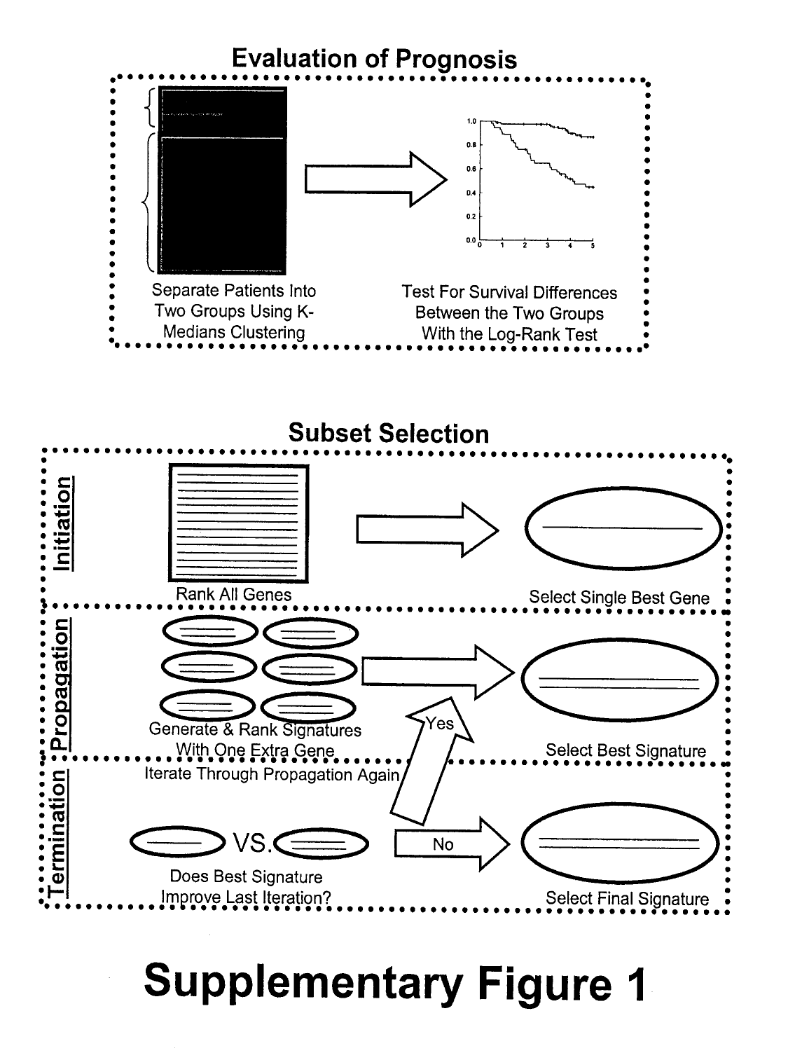

Several smaller expression studies of non-small cell lung cancer were also available but, because of their limited number of patients, were not useful as validation datasets. To leverage these resources, we combined all patients from the four studies described above, along with datasets from the Mayo Clinic and Washington University (39), and two additional studies of mRNA expression in NSCLC (40, 41). In each of these cases, the raw data (CEL files) was downloaded and pre-processed using the RMA algorithm (42) as implemented in the affy package (43) (v1.6.7) for R (v2.1.1). One dataset (40) included highly-correlated technical replicates for some samples, which were collapsed through ProbeSet-wise averaging. The resulting dataset of 589 patients was then subject to the same nearest-centre classification described above. Survival between the two groups was tested using Cox proportional hazards model with adjustment for stage. The normalized data and clinical annotations for all patients used in this paper are presented in FIG. 5.

### Permutation and Enrichment Analysis

To determine the number of 6-gene classifiers (signatures) that could be generated from our 158-gene training dataset we performed a permutation analysis. We tested the prognostic capability of all combinations of ten million combinations of six genes. For each combination we divided the patients into two groups using k-means clustering and calculated significance using log-rank analysis.

Study of all combinations is not possible for larger subset sizes because of the combinatorial explosion. This analysis was performed in the R statistical environment (v2.6.1) using the survival package (v2.34).

To test each signature we used the clusters defined in our training cohort to classify patients from four additional datasets (36, 37, 40, 41), again using Euclidean distances and log-rank analysis. The normalized data for each of these datasets was extracted for the genes in each signature. Euclidean distances were calculated between each patient and the centre of the two training clusters, and the patient was classified into the nearest cluster. Survival differences between good and poor prognosis clusters were then assessed using log-rank analysis.

Finally, to consider the generalizability of each prognostic signature across all four testing datasets we employed percentile analysis. The distribution of subsets with prognostic significance (χ²>3.84 or p<0.05) in the training dataset was visualized using Gaussian density plots. First, for visualization purposes we calculated and plotted the Gaussian kernel density of prognostic signatures in each validation dataset. Next, we calculated the percentile rank of each signature in each of the four validation datasets. The product of these ranks provides an estimate of the overall validation of a classifier across all four datasets, and we plotted the Gaussian kernel densities of these ranks. The performance of the six-gene mSD-signature was then treated in the same manner and its location marked on plots with an arrow to indicate its performance relative to the distribution of all potential prognostic markers.

Specifically, we focused on those six-gene signatures having a p-value below 0.05 (a strength greater than pre-defined parameter). Enrichment of each gene was studied in the high-strength (p<0.05) subsets using two enrichment statistics. First, the fraction of subsets containing that gene that were statistically significant at p<0.05 by a log-rank test was calculated. Second, this fraction was compared to the fraction that would be expected by chance alone using a bootstrap analysis. A bootstrap analysis involves repeated random-samplings from the original dataset, in this case 1000 random samplings were used to estimate each p-value. Bootstrap analysis is preferred when the distribution of the underlying data is unknown or highly complex.

Genes were ranked by the p-value-based enrichment statistics. To identify genes that have an enrichment above a pre-defined threshold we set our threshold as p<0.01.

### Results

### Classifier Training

To determine the impact of alternative statistical methods on prognostic marker identification we considered our previously published 147-patient, 158-gene RT-PCR NSCLC dataset. This dataset had been analyzed with a risk-score methodology, which identified a three-gene classifier capable of separating patients into groups with significantly different prognoses (19). The majority of signatures developed for NSCLC employed linear or risk-score methods to classify patients (11, 13, 14, 16, 23), which are unable to capture non-linear interactions amongst genes. For example, regulatory networks make substantial use of “or” logic: a cell may respond to hypoxic conditions by up-regulating HIF1A or down-regulating VHL. Such relationships cannot generally be captured by linear methods. We thus developed a novel non-linear semi-supervised method by coupling unsupervised pattern-recognition to gradient descent optimization (i.e. mSD). Referring to FIG. 5, the modified steepest-descent algorithm has two components: a prognosis-prediction component and a feature-selection component. First, given a set of one or more features, mSD estimates prognosis in a semi-supervised way. Patients are clustered using k-medians clustering into two groups and the survival difference between these two groups is measured with the chi-squared output of a log-rank test. Features are ranked according to this chi-squared statistic. Second, features are selected using a gradient-descent approach. The initial feature is chosen based on the univariate ranking of all features. Following this initiation phase, features are added one-by-one by greedy descent. Once a local minimum has been reached, the algorithm terminates.

Applying mSD to a training dataset of 147 NSCLC patients initially generated a prognostic signature comprising six genes: syntaxin 1A (STX1A), hypoxia inducible factor 1A (HIF1A), chaperonin containing TCP1 subunit 3 (CCT3), MHC Class II DPbeta 1 (HLA-DPB1), v-maf musculoaponeurotic fibrosarcoma oncogene homolog K (MAFK), and ring finger protein 5 (RNF5) (as described in U.S. patent application Ser. No. 11/940,707). Table 1 gives additional information on these genes. Specifically, stable (Entrez Gene ID) identifiers and the independent univariate prognostic ability (based on the log-rank test and Cox proportional hazards modeling) are given for each component of the six-gene mSD signature.

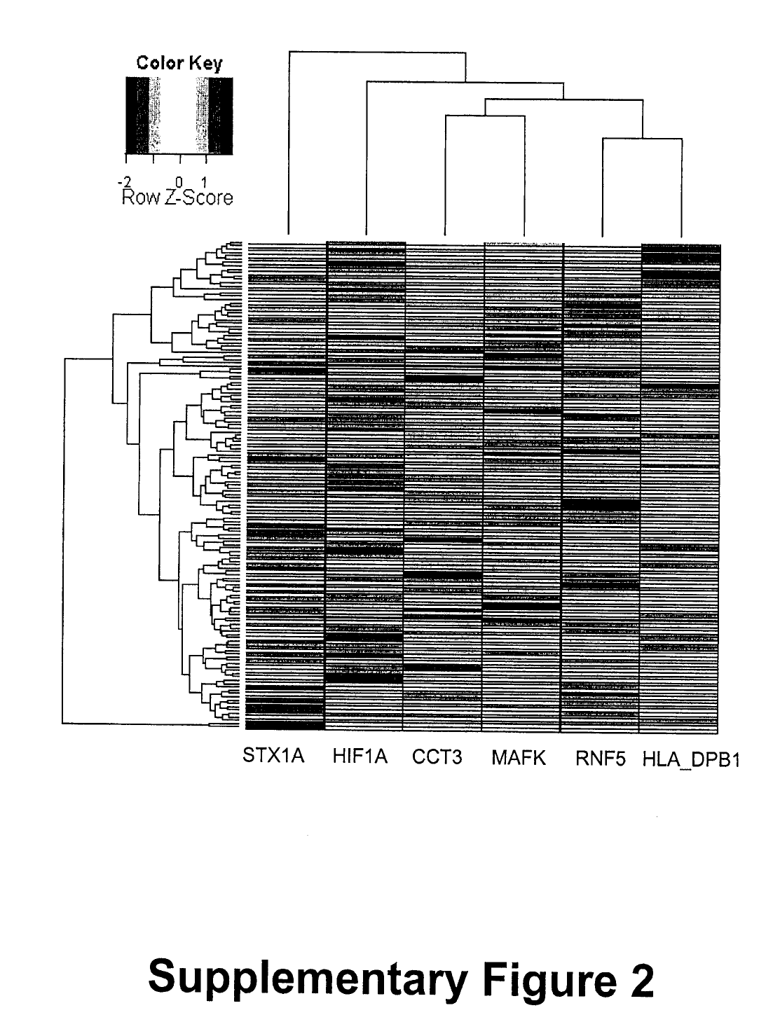

Referring to FIG. 6, we visualized the aforementioned 6-gene mSD signature using unsupervised pattern-recognition and found that the six genes were largely uncorrelated. The expression profiles of the six-genes from the mSD-signature for the 147 patients of the training dataset were subjected to unsupervised pattern-recognition. Agglomerative hierarchical clustering using complete linkage was performed. The columns represent genes and the rows represent individual patients. The six genes all show unique expression patterns, as indicated by the long terminal arms of the column dendrogram. Patients do not fall into one or two large clusters, but rather into a diversity of small, non-linear ones, as indicated by the row dendrogram.

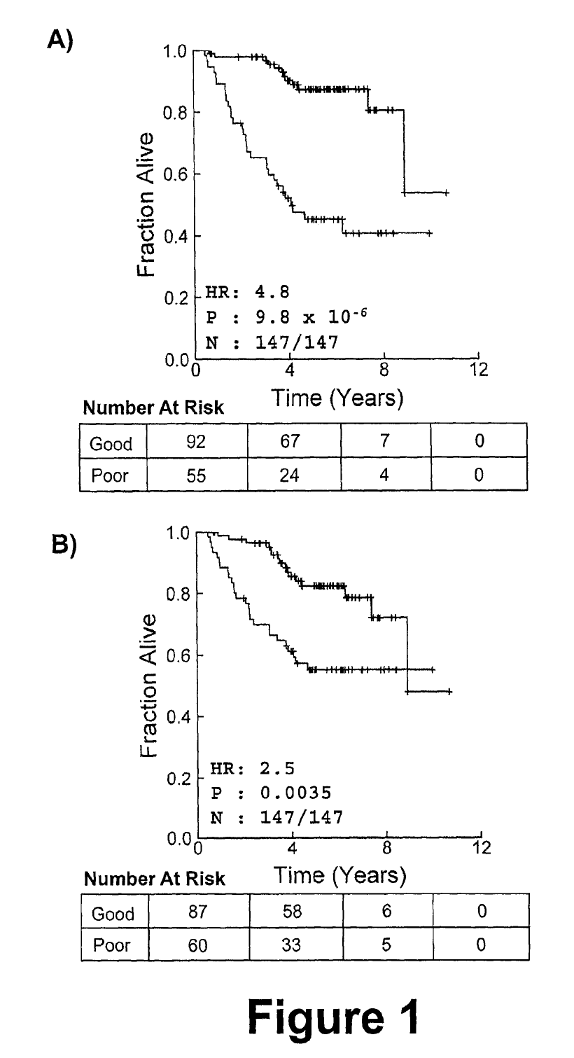

The signature separated the 147 training patients into groups with significantly different survivals (p=2.14×10⁻⁸; log-rank test; FIG. 1A). Both patient prognosis and treatment are strongly affected by clinical stage, and our previous analysis showed it to be a significant covariate in the training dataset (19). Accordingly, we adjusted for the effects of stage using Cox proportional hazards modeling and showed that the 6-gene mSD molecular signature was independent of clinical stage (HR 4.8, p<0.001). We also performed a preliminary validation using leave-one-out cross-validation (24). The aforementioned six-gene signature divided patients into two groups with significantly different outcome during cross-validation (FIG. 1B, HR: 2.5, p=0.0036). Referring to Table 2, the six-gene signature leads to similar patient classifications in the training dataset as our earlier three-gene signature. Table 2 shows the survival, clinical stage, and normalized expression levels for the six-gene signature of all patients considered in any analysis in this study. Patients are identified by the study of origin: UHN, Lau et al.; MI02, Beer et al.; MIT, Bhattacharjee et al.; Duke, Potti et al.; MI06, Raponi et al.; AD1, Larsen et al.; SQ2, Larsen et al.; LuMayo and LuWashU, Lu et al. mSD prediction status is also given for the training (UHN) dataset.

### Classifier Validation

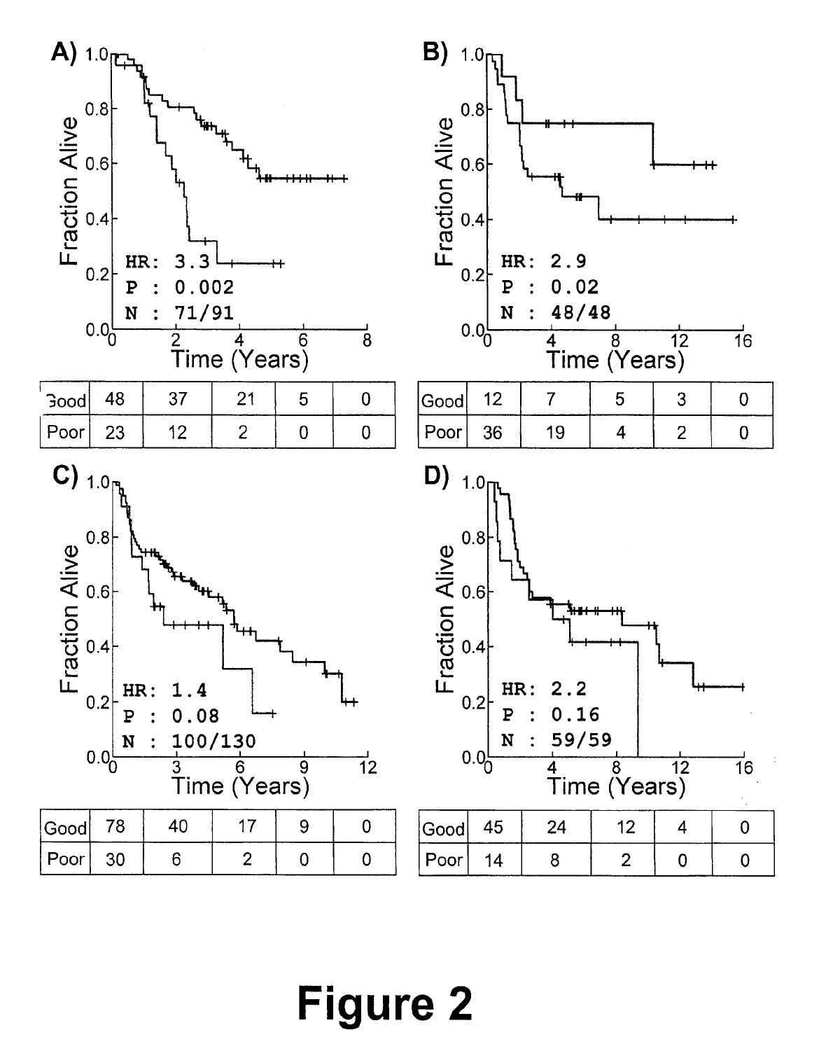

To validate our initial six-gene signature we tested its ability to stratify patients into groups with different prognosis using four independent publicly available datasets from Duke University (25), the University of Michigan (16), and the Prince Charles Hospital (13, 14). These datasets represent two versions of Affymetrix arrays (U133Plus2.0, Duke; U133A, Michigan) and a custom cDNA array (Prince Charles). Two of these studies comprise exclusively squamous cell carcinomas (13, 16), one exclusively adenocarcinomas (14), and one both (25). Each dataset was analyzed separately, as outlined in the supplementary methods. The molecular stratifications are plotted in FIG. 1. The six-gene signature was prognostic in all four independent patient cohorts, with hazard ratios ranging from 1.4 (p=0.08) to 3.3 (p=0.002). The validation on the two datasets from Prince Charles is notable because one gene from our six-gene signature (RNF5) and two of the four normalization genes were not present on the array platform. Despite this missing information, the mSD signature classified patients into groups with significantly different outcomes (FIGS. 2B and 2D). In the two Affymetrix datasets (FIGS. 2A and 2C) approximately 10% of patients had expression profiles equidistant from the two training clusters. These patients were not classified; in practice these equivocal classifications would be assigned to standard clinical practice.

### Pooled Validation

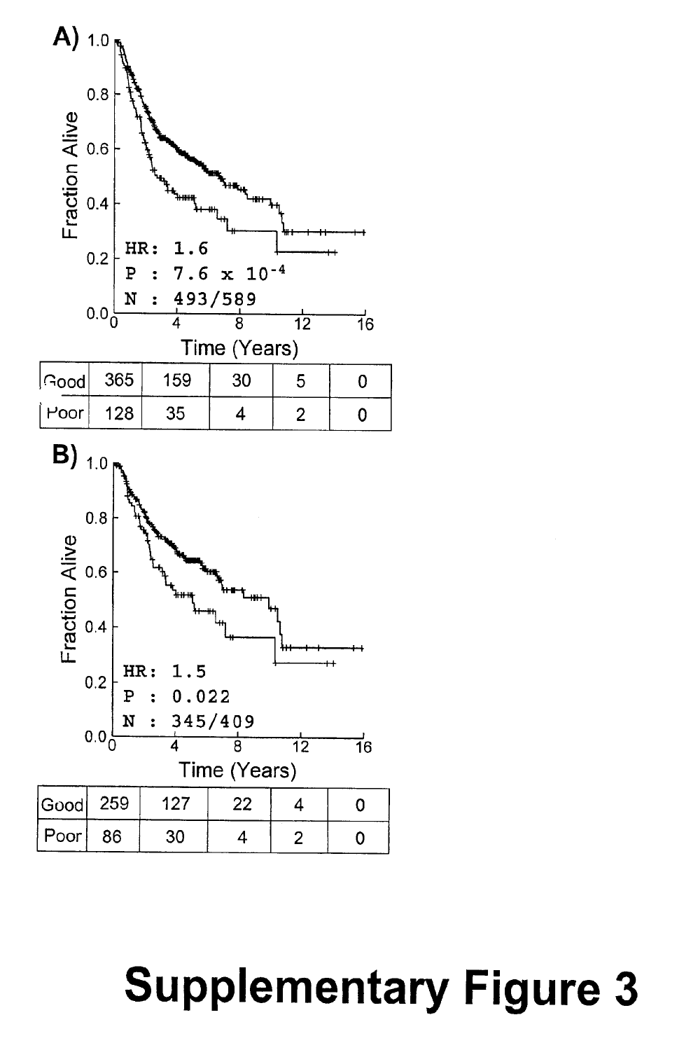

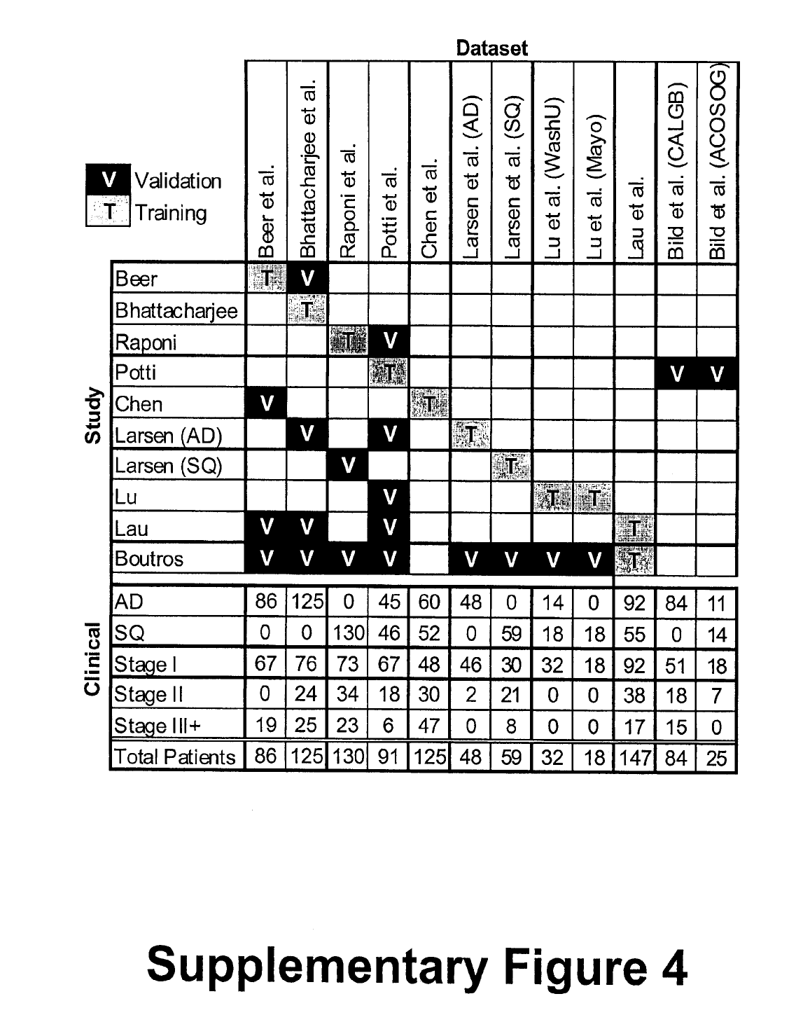

In addition to the four datasets analyzed in FIG. 1, a number of small or older NSCLC datasets exist. We combined the data from the four validation datasets with that from a previous study of adenocarcinomas on the older Hu6800 Affymetrix array (11), a study of adenocarcinomas on the relatively old U95Av2 Affymetrix array (12), and small adenocarcinoma and squamous cell carcinoma datasets on Affymetrix U133A arrays from a pooled study (23). This generated a cohort of 589 patients taken from 8 datasets. This cohort was separated into two groups using the aforementioned six-gene signature (FIG. 7A). The resulting groups showed significant stage-adjusted differences in survival with a hazard ratio of 1.6 (95% CI 1.2-2.2; p=7.6×10⁻⁴). The six-gene signature was also capable of separating Stage I patients from this cohort into two groups with different survival (FIG. 7B), with a hazard ratio of 1.5 (95% CI 1.1 to 2.2; p=0.02). These results for Stage I patients were adjusted for clinical stage (IA vs. IB), demonstrating that our molecular classification improves upon existing staging criteria. The hazard ratios in this pooled analysis are somewhat compressed by the addition of older and less-sensitive microarray platforms, but nevertheless the results are statistically significant consistent in a very large patient cohort. The extensive validation of this initial six-gene signature compares favorably to other published NSCLC signatures (FIG. 8). Table 3 summarizes all validation datasets.

### Permutation and Enrichment Analysis

We identified a six-gene classifier that shows partial overlap with the three-gene classifier identified previously from the same training dataset using risk-score methods. We questioned whether other small prognostic signatures could be identified from this 158-gene dataset. To test this question comprehensively we mapped our 158 genes into four test datasets (11, 12, 16, 25). In total 113 genes were common to these four datasets, and adding additional datasets greatly reduced this number. We restricted subsequent analyses to the 113 genes profiled in all four datasets. We then generated ten million permutations of six genes and tested their prognostic capability in these four datasets. For each subset we calculated its statistical significance using the log-rank test, as before.

A large number of these permutations showed statistical significance. In total 16.4% of all six-gene signatures were significant at p<0.05. This is 3.28-fold greater than the 5% expected by chance alone, and reflects a statistically significant enrichment (p<2.2×10⁻¹⁶; proportion test).

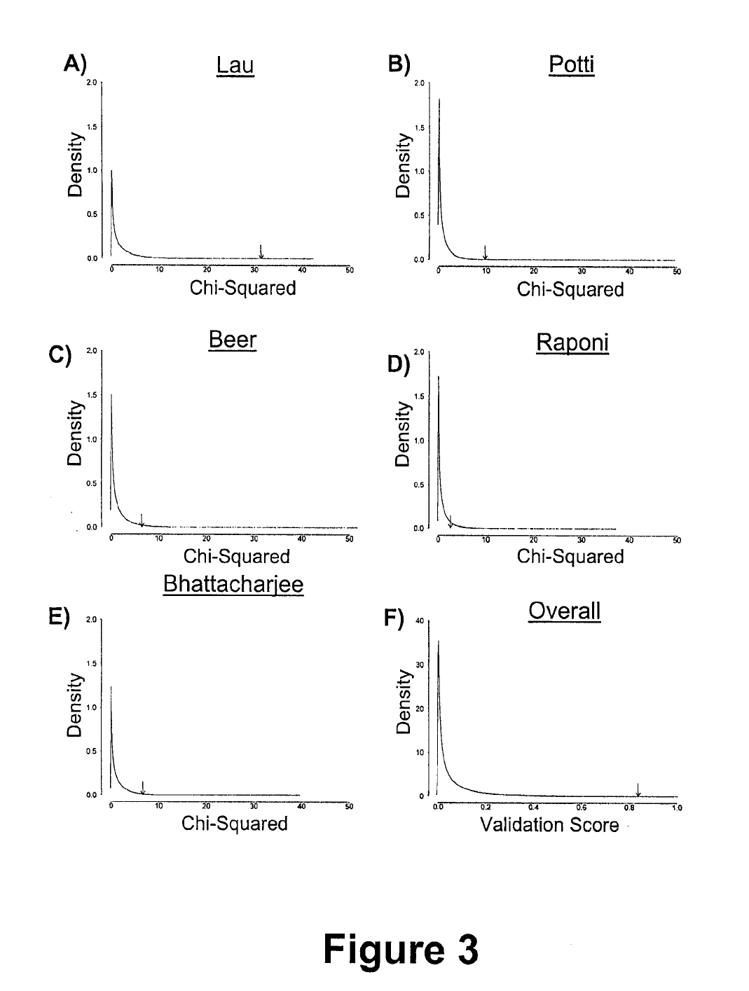

The distribution of all 10,000,000 six-gene signatures is shown in FIG. 3A as a kernel density estimate. Kernel density estimates are an established method of estimating the probability density function of a random variable. They can be thought of as smoothed histograms, where the y-axis reflects the likelihood of observing the value specified by the x-axis. In FIG. 3A the x-axis indicates the chi-squared value from the log-rank analysis. The higher the chi-squared the smaller (more significant) the p-value for differential prognosis between the two predicted groups. Thus, more effective prognostic signatures lie to the right of the plot.

We next compared the validation of the aforementioned 6-gene mSD signature to that of ten million random 6-gene signatures. For each test dataset (11, 12, 16, 25) the distribution of validation rates was again plotted as kernel density estimates. For each kernel density estimate in the training dataset we marked the performance of the six-gene mSD signature in that dataset with an arrow (FIGS. 3B-E). The mSD signature performs well in each of the four datasets, but with some variability. The lower bound was the squamous cell carcinoma dataset reported by Raponi et al. where our classifier was amongst the top 10.4% of all signatures. The upper bound was the dataset reported by Potti and coworkers where it was amongst the top 0.14% of all signatures. Summary data from all permutation analyses are presented in Table 4.

These data demonstrate the efficacy of the aforementioned initial six-gene signature in four distinct testing datasets. While said 6-gene signature performed amongst the top 10% of all signatures in each test dataset, it was not the single best signature in any single dataset. Rather, its strength is its validation in four independent datasets. To compare the validation of this 6-gene signature across all four test datasets we calculated its percentile ranking in each dataset and took the product of these rankings. The resulting validation score provides a measure of the inter-dataset reproducibility of a signature. Only 1,789 of the 10,000,000 signatures tested perform better than the mSD signature across all four validation datasets. Thus the mSD signature was superior to 99.98% of signatures tested (FIG. 3F). The small difference in performance of the mSD signature in the training and testing datasets (99.999% vs. 99.982%) indicates minimal over-fitting on our training dataset.

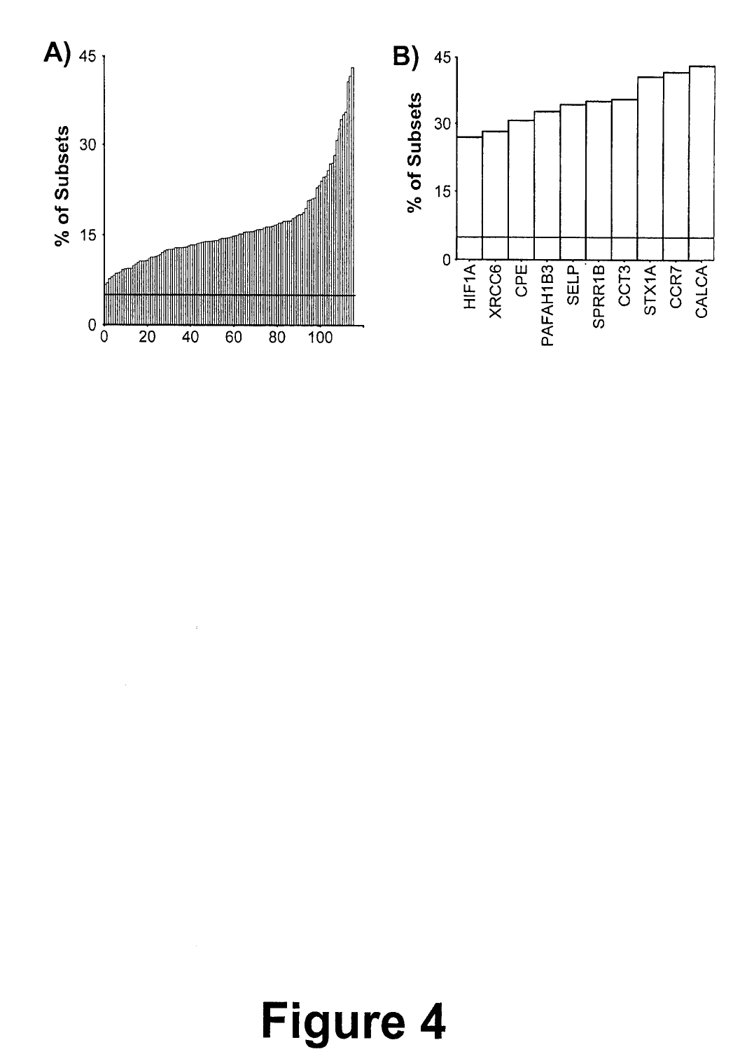

Having used our large permutation dataset to rank the aforementioned initial six-gene prognostic signature, we next tested if specific genes were enriched in prognostic signatures. For each gene, we calculated the percentage of signatures containing it that were statistically significant (p<0.05, log-rank test). At this threshold we expect 5% of signatures to be significant by chance alone. When we plotted the percentages for the 113 gene set (FIG. 4A), most genes were enriched over this baseline, with enrichment values ranging from 6.7% to 43.1%. This likely reflects the enrichment of our test dataset for putative prognostic genes (19).

Table 5 provides the enrichment values for all 113 genes. At an enrichment above a threshold set at p<0.01, 16-genes remain in our final signature. This choice of threshold is further supported by the clear inflection point that is evident both in the enrichment plot (FIG. 4A) and in the list of p-values (Table 5) between the 16th and 17th gene, where p-values drop by an order of magnitude (from 2.13e-4 to 6.70e-2). This inflection point, combined with matching the traditional p-value thresholds of p<0.05 and p<0.01, provides support for the threshold that creates a final gene signature selected from these 16 genes.

FIG. 4B shows further focus on the ten most highly enriched genes. Both genes shared by the aforementioned 6-gene mSD signature and the previously identified risk-score 3-gene signature are present on this list (STX1A, 3^{rd}, and HIF1A, 10^{th}), as are one additional gene from the mSD signature (CCT3, 4^{th}) and one additional gene from the risk-score signature (CCR7, 2^{nd}). Genes on this list are highly effective in prognostic signatures, independent of the other genes they are combined with, and may therefore represent unique aspects of disease initiation or progression.

### Summary

The observed lack of overlap in typically reported prognostic signatures for NSCLC likely results from the use of different statistical techniques. To address this, we trained two distinctive algorithms on a single dataset to determine if identical signatures would be found. For training, we selected a real-time PCR dataset of 158 genes assessed in 147 patients, which we had used previously to identify a three-gene signature using linear risk-score methods (19). To provide a counterpoint to this linear analysis we then developed a semi-supervised algorithm by coupling unsupervised pattern-recognition and gradient descent algorithms (i.e. mSD).

The application of mSD to the same 147-patient training dataset identified a six-gene signature. This signature stratified NSCLC patients into two groups with different outcomes in four independent public datasets (FIG. 1). These datasets included three different array platforms and both squamous cell carcinoma and adenocarcinoma patients. Beyond these validation datasets, a number of other smaller or older studies exist. We combined four such datasets with the four validation datasets to generate a cohort of 589 patients drawn from 8 published studies. The initial six-gene signature performed well, both on the entire cohort (FIG. 2A) and when Stage I patients are considered separately (FIG. 2B). This suggests that said signature may identify a cohort of Stage I patients who have the potential to benefit from adjuvant therapy. Importantly, all validations include adjustments for clinical stage, indicating that our signature is independent of traditional staging criteria, which remain the standard method for determining treatment and predicting outcome, although other factors such as age and grade also play roles.

Clinical implementation of signatures should be straight-forward. For each patient, RT-PCR analysis would be performed for the identified prognostic genes in conjunction with a number of (i.e. 4) house-keeping genes for normalization purposes. Following normalization, Euclidean distances will determine if a patient's profile most resembles good or poor prognosis tumors—a similar approach to that adopted in two major breast-cancer studies (26, 27). Such signature(s) can be used even if some of the PCR reactions fail or data is otherwise unavailable, as shown by successful validation of the aforementioned 6-gene signature in two cDNA microarray datasets where one signature and one normalization gene were not present on the array platform (13, 14).

We have validated the aforementioned six-gene signature in eight of the eleven most recent NSCLC microarray studies (FIG. 8). The eight included studies are themselves quite heterogeneous, with differences in both clinical and technical covariates. Clinically, the studies had varying patient-inclusion criteria, with some studies including patients of only some stages (11, 23) or histologies (11-14). Technically, studies varied in the fraction of tumour sample included in each sample, the protocols used to extract RNA and the microarray platforms used to assess mRNA levels. The ability of the aforementioned six-gene signature to handle these many confounding factors may reflect both our secondary-validation design (19) and the non-linear nature of the mSD algorithm. The three omitted studies include one where the raw array data has not yet been deposited in a public database (18) and two where identifiers to link the expression data to clinical covariates do not appear to have been provided (15). This extensive validation was only possible because of the public availability of a large number of previous studies, highlighting the benefit of earlier work in the field.

Two genes (STX1A and HIF1A) are common to both the previously described three-(19) and aforementioned six-gene signatures. This partial overlap led us to hypothesize that additional small prognostic signatures could be identified from our training dataset. To test this, we trained ten million sets of six genes in our PCR dataset and tested each in four independent validation datasets. In both the training and testing datasets the aforementioned six-gene signature is superior to 99.98% of prognostic signatures (FIG. 3F). This provides justification and verification of the universality of our method for identifying and evaluating prognostic signatures and of the underlying approaches (and algorithms) used to generate the signatures.

These results demonstrate that very large numbers of potential prognostic signatures exist. Our permutation study focused on 113 genes that were profiled in five separate studies. This small dataset can generate approximately 2.5-billion unique six-gene signatures. If, as our results suggest, 0.02% of these can be verified in multiple independent validation cohorts, then a minimum of 500,000 verifiable six-gene prognostic signatures exist. This large number may explain the poor gene-wise overlap observed in prognostic signatures from different groups (19). It will be critical to determine if this conclusion can be generalized to other datasets and sizes of prognostic signature.

A detailed comparison of verifiable prognostic signatures might reveal common features. Our initial univariate shows that some specific genes were highly enriched in statistically significant prognostic signatures (FIG. 4B). In particular, signatures containing calcitonin-related polypeptide alpha were statistically significant 43% of the time, implicating it in disease etiology. Overall, three genes in the mSD signature were enriched in prognostic signatures. Additional study of verifiable prognostic signatures might reveal other such insights. For example, certain pathways might be captured by all signatures, but represented by a number different of genes. Gene-gene interactions could be determined from pairs of genes co-occurring at a high frequency.

Our approach may provide a template for future studies to develop reproducible, mRNA-based signatures for cancer and other complex diseases. We started by using a high-quality training dataset enriched for prognostic markers. By keeping this dataset small we minimize the problems of over-fitting that arise from using thousands of genes. Next, we used a non-linear algorithm that dynamically learned patient groupings (i.e. a semi-supervised algorithm). Finally, we extensively validated our results, using cross-validation, multiple external datasets, and permutation-type analyses. Application of this protocol to the development of other signatures should be fruitful.

In summary, the present application encompasses a novel, semi-supervised algorithm (utilized in combination with a novel permutation analysis) which was used to demonstrate that a single training dataset can yield multiple prognostic signatures. By way of example, an initial (and previously described; i.e. U.S. patent application Ser. No. 11/940,707)) was validated in multiple testing datasets. Additionally, the application further teaches an approach for the identification and verification of a multiplicity of diverse and distinct NSCLC prognostic gene signatures, as exemplified by those signatures comprising at least three of CALCA, CCR7, STX1A, CCT3, SPRR1B, SELP, PAFAH1B3, CPE, XRCC6, HIF1A, MARCH6, PLOD2, NAP1L1, SFTPC, KRT5 and STC1.

Although preferred embodiments of the invention have been described herein, it will be understood by those skilled in the art that variations may be made thereto without departing from the spirit of the invention or the scope of the appended claims.

### REFERENCES

1. Tsuboi, M., Ohira, T., Saji, H., Miyajima, K., Kajiwara, N., Uchida, O., Usuda, J. & Kato, H. (2007) Ann Thorac Cardiovasc Surg 13, 73-7.
2. Mountain, C. F. (2002) Clin Chest Med 23, 103-21.
3. Mountain, C. F. (1997) Chest 111, 1710-7.
4. Jones, K. L. & Buzdar, A. U. (2004) Endocr Relat Cancer 11, 391-406.
5. Zaniboni, A. & Labianca, R. (2004) Ann Oncol 15, 1310-8.
6. Gramont, A. (2005) Semin Oncol 32, 11-4.
7. Group”, N.-s. C. L. C. C. (1995) BMJ 311, 899-909.
8. Winton, T., Livingston, R., Johnson, D., Rigas, J., Johnston, M., Butts, C., Cormier, Y., Goss, G., Inculet, R., Vallieres, E., Fry, W., Bethune, D., Ayoub, J., Ding, K., Seymour, L., Graham, B., Tsao, M. S., Gandara, D., Kesler, K., Demmy, T. & Shepherd, F. (2005) N Engl J Med 352, 2589-97.
9. Douillard, J. Y., Rosell, R., De Lena, M., Carpagnano, F., Ramlau, R., Gonzales-Larriba, J. L., Grodzki, T., Pereira, J. R., Le Groumellec, A., Lorusso, V., Clary, C., Tones, A. J., Dahabreh, J., Souquet, P. J., Astudillo, J., Fournel, P., Artal-Cortes, A., Jassem, J., Koubkova, L., His, P., Riggi, M. & Hurteloup, P. (2006) The lancet oncology 7, 719-27.
10. Kato, H., Ichinose, Y., Ohta, M., Hata, E., Tsubota, N., Tada, H., Watanabe, Y., Wada, H., Tsuboi, M., Hamajima, N. & Ohta, M. (2004) N Engl J Med 350, 1713-21.
11. Beer, D. G., Kardia, S. L., Huang, C. C., Giordano, T. J., Levin, A. M., Misek, D. E., Lin, L., Chen, G., Gharib, T. G., Thomas, D. G., Lizyness, M. L., Kuick, R., Hayasaka, S., Taylor, J. M., Iannettoni, M. D., Orringer, M. B. & Hanash, S. (2002) Nat Med 8, 816-24.
12. Bhattacharjee, A., Richards, W. G., Staunton, J., Li, C., Monti, S., Vasa, P., Ladd, C., Beheshti, J., Bueno, R., Gillette, M., Loda, M., Weber, G., Mark, E. J., Lander, E. S., Wong, W., Johnson, B. E., Golub, T. R., Sugarbaker, D. J. & Meyerson, M. (2001) Proc Natl Acad Sci USA 98, 13790-5.
13. Larsen, J. E., Pavey, S. J., Passmore, L. H., Bowman, R., Clarke, B. E., Hayward, N. K. & Fong, K. M. (2007) Carcinogenesis 28, 760-6.
14. Larsen, J. E., Pavey, S. J., Passmore, L. H., Bowman, R. V., Hayward, N. K. & Fong, K. M. (2007) Clin Cancer Res 13, 2946-54.
15. Potti, A., Mukherjee, S., Petersen, R., Dressman, H. K., Bild, A., Koontz, J., Kratzke, R., Watson, M. A., Kelley, M., Ginsburg, G. S., West, M., Harpole, D. H., Jr. & Nevins, J. R. (2006) N Engl J Med 355, 570-80.
16. Raponi, M., Zhang, Y., Yu, J., Chen, G., Lee, G., Taylor, J. M., Macdonald, J., Thomas, D., Moskaluk, C., Wang, Y. & Beer, D. G. (2006) Cancer Res 66, 7466-72.
17. Sun, Z., Wigle, D. A. & Yang, P. (2008) J Clin Oncol 26, 877-83.
18. Chen, H. Y., Yu, S. L., Chen, C. H., Chang, G. C., Chen, C. Y., Yuan, A., Cheng, C. L., Wang, C. H., Terng, H. J., Kao, S. F., Chan, W. K., Li, H. N., Liu, C. C., Singh, S., Chen, W. J., Chen, J. J. & Yang, P. C. (2007) N Engl J Med 356, 11-20.
19. Lau, S. K., Boutros, P. C., Pintilie, M., Blackhall, F. H., Zhu, C. Q., Strumpf, D., Johnston, M. R., Darling, G., Keshavjee, S., Waddell, T. K., Liu, N., Lau, D., Penn, L. Z., Shepherd, F. A., Jurisica, I., Der, S. D. & Tsao, M. S. (2007) Clin Oncol 25, 5562-9.
20. Ein-Dor, L., Zuk, O. & Domany, E. (2006)Proc Natl Acad Sci USA 103, 5923-8.
21. Bachtiary, B., Boutros, P. C., Pintilie, M., Shi, W., Bastianutto, C., Li, J. H., Schwock, J., Zhang, W., Penn, L. Z., Jurisica, I., Fyles, A. & Liu, F. F. (2006) Clin Cancer Res 12, 5632-40.
22. Blackhall, F. H., Pintilie, M., Wigle, D. A., Jurisica, I., Liu, N., Radulovich, N., Johnston, M. R., Keshavjee, S. & Tsao, M. S. (2004) Neoplasia 6, 761-7.
23. Lu, Y., Lemon, W., Liu, P. Y., Yi, Y., Morrison, C., Yang, P., Sun, Z., Szoke, J., Gerald, W. L., Watson, M., Govindan, R. & You, M. (2006) PLoS Med 3, e467.
24. Simon, R., Radmacher, M. D., Dobbin, K. & McShane, L. M. (2003) J Natl Cancer Inst 95, 14-8.
25. Bild, A. H., Yao, G., Chang, J. T., Wang, Q., Potti, A., Chasse, D., Joshi, M. B., Harpole, D., Lancaster, J. M., Berchuck, A., Olson, J. A., Jr., Marks, J. R., Dressman, H. K., West, M. & Nevins, J. R. (2006) Nature 439, 353-7.
26. van de Vijver, M. J., He, Y. D., van't Veer, L. J., Dai, H., Hart, A. A., Voskuil, D. W., Schreiber, G. J., Peterse, J. L., Roberts, C., Marton, M. J., Parrish, M., Atsma, D., Witteveen, A., Glas, A., Delahaye, L., van der Velde, T., Bartelink, H., Rodenhuis, S., Rutgers, E. T., Friend, S. H. & Bernards, R. (2002) N Engl J Med 347, 1999-2009.
27. van't Veer, L. J., Dai, H., van de Vijver, M. J., He, Y. D., Hart, A. A., Mao, M., Peterse, H. L., van der Kooy, K., Marton, M. J., Witteveen, A. T., Schreiber, G. J., Kerkhoven, R. M., Roberts, C., Linsley, P. S., Bernards, R. & Friend, S. H. (2002) Nature 415, 530-6.
28. Barsyte-Lovejoy, D., Lau, S. K., Boutros, P. C., Khosravi, F., Jurisica, I., Andrulis, I. L., Tsao, M. S. & Penn, L. Z. (2006) Cancer Res 66, 5330-7.
29. Duda, R. O., Hart, P. E. & Stork, D. G. (2001) Pattern classcation (Wiley, New York).
30. Boutros P C & Okey A B (2005) Unsupervised pattern recognition: an introduction to the whys and wherefores of clustering microarray data. Brief Bioinform. 6(4):331-343.
31. Anonymous (1995) Chemotherapy in non-small cell lung cancer: a meta-analysis using updated data on individual patients from 52 randomised clinical trials. Non-small Cell Lung Cancer Collaborative Group. (Translated from eng) Bmj 311(7010):899-909 (in eng).
32. de Hoon M J, Imoto S, Nolan J, & Miyano S (2004) Open source clustering software. Bioinformatics 20(9): 1453-1454.
33. Lau S K, et al. (2007) Three-gene prognostic classifier for early-stage non small-cell lung cancer. J Clin Oncol 25(35):5562-5569.
34. Larsen J E, et al. (2007) Gene expression signature predicts recurrence in lung adenocarcinoma. (Translated from eng) Clin Cancer Res 13(10):2946-2954 (in eng).
35. Larsen J E, et al. (2007) Expression profiling defines a recurrence signature in lung squamous cell carcinoma. (Translated from eng) Carcinogenesis 28(3):760-766 (in eng).
36. Bild A H, et al. (2006) Oncogenic pathway signatures in human cancers as a guide to targeted therapies. Nature 439(7074):353-357.
37. Raponi M, et al. (2006) Gene expression signatures for predicting prognosis of squamous cell and adenocarcinomas of the lung. (Translated from eng) Cancer Res. 66(15):7466-7472 (in eng).
38. Barsyte-Lovejoy D, et al. (2006) The c-Myc oncogene directly induces the H19 noncoding RNA by allele-specific binding to potentiate tumorigenesis. (Translated from eng) Cancer Res. 66(10):5330-5337 (in eng).
39. Lu Y, et al. (2006) A gene expression signature predicts survival of patients with stage I non-small cell lung cancer. (Translated from eng) PLoS Med 3(12):e467 (in eng).
40. Bhattacharjee A, et al. (2001) Classification of human lung carcinomas by mRNA expression profiling reveals distinct adenocarcinoma subclasses. Proc. Natl. Acad. Sci. U.S.A. 98(24):13790-13795.
41. Beer D G, et al. (2002) Gene-expression profiles predict survival of patients with lung adenocarcinoma. Nat Med 8(8):816-824.
42. Irizarry R A, et al. (2003) Summaries of Affymetrix GeneChip probe level data. Nucleic Acids Res. 31(4):e15.
43. Gautier L, Cope L, Bolstad B M, & Irizarry R A (2004) affy—analysis of Affymetrix GeneChip data at the probe level. Bioinformatics 20(3):307-315.

TABLE 1
Properties of the Six-Gene Signature

| Gene | Entrez | | | | |
|---|---|---|---|---|---|
| Symbol | Gene ID | Gene annotation | HR* | 95% CI | P |
| STX1A | 6804 | syntaxin 1A (brain) | 1.6 | 1.3-2.1 | <0.001 |
| HIF1A | 3091 | hypoxia-inducible | 1.4 | 1.1-1.7 | 0.007 |
| | | factor 1 alpha | | | |
| CCT3 | 7203 | chaperonin containing | 1.9 | 1.3-2.6 | <0.001 |
| | | TCP1, subunit 3 | | | |
| HLA- | 3115 | MHC Class II, DPbeta | 0.75 | 0.59-1.0 | 0.019 |
| DBPB1 | | 1 | | | |
| MAFK | 7375 | v-maf | 1.1 | 0.82-1.5 | 0.45 |
| | | musculoaponeurotic | | | |
| | | fibrosarcoma oncogene | | | |
| | | homolog K (avian) | | | |
| RNF5 | 6048 | ring finger protein 5 | 1.2 | 0.92-1.6 | 0.18 |

*HR denotes hazard ratios for death; CI denotes confidence interval. P values were determined by the log-rank test. All survival data is from the Lau et al dataset.

TABLE 2

| | | | | | surv | surv | | df | | |
|---|---|---|---|---|---|---|---|---|---|---|
| Study | ID | Histology | stage | stage 2 | time | stat | df time | stat | Ras | STX1A |
| UHN | B007 | AD | 1B | I | 6.153 | 0 | 6.153 | 0 | NA | −2.376 |
| UHN | B013 | AD | 2B | II | 3.970 | 0 | 3.970 | 0 | NA | 2.166 |
| UHN | B019 | SQ | 2B | II | 4.233 | 0 | 4.233 | 0 | NA | −1.021 |
| UHN | B033 | AD | 1B | I | 3.838 | 0 | 3.838 | 0 | NA | −1.342 |
| UHN | B048 | AD | 1B | I | 3.781 | 0 | 3.781 | 0 | NA | 0.205 |
| UHN | B067 | AD | 1B | I | 3.625 | 0 | 3.625 | 0 | NA | −2.509 |
| UHN | B084 | AD | 2B | II | 4.044 | 0 | 4.044 | 0 | NA | 0.378 |
| UHN | L005 | AD | 1A | I | 7.227 | 0 | 7.227 | 0 | NA | 0.089 |
| UHN | L009 | AD | 1B | I | 7.381 | 0 | 7.381 | 0 | NA | −1.498 |
| UHN | L012 | AD | 2B | II | 6.726 | 0 | 6.726 | 0 | NA | −0.318 |
| UHN | L018 | AD | 1B | I | 7.236 | 0 | 7.236 | 0 | NA | −0.695 |
| UHN | L023 | SQ | 2B | II | 4.197 | 1 | 1.112 | 1 | NA | −0.513 |
| UHN | L027 | SQ | 1B | I | 8.241 | 0 | 8.241 | 0 | NA | −1.316 |
| UHN | L028 | SQ | 1B | I | 3.770 | 1 | 3.241 | 1 | NA | −0.132 |
| UHN | L030 | AD | 2B | II | 2.222 | 1 | 1.534 | 1 | NA | 0.744 |
| UHN | L047 | AD | 3A | III+ | 3.395 | 1 | 2.496 | 1 | NA | 0.730 |
| UHN | L049 | AD | 3A | III+ | 6.277 | 1 | 6.230 | 1 | NA | 1.480 |
| UHN | L051 | SQ | 2B | II | 3.438 | 0 | 3.438 | 0 | NA | −1.603 |
| UHN | L052 | AD | 3A | III+ | 4.175 | 1 | 3.948 | 1 | NA | 0.754 |
| UHN | L056 | SQ | 1B | I | 5.995 | 0 | 5.995 | 0 | NA | −1.777 |
| UHN | L058 | AD | 2B | II | 7.915 | 0 | 7.915 | 0 | NA | 1.503 |
| UHN | L059 | AD | 2A | II | 6.151 | 0 | 6.151 | 0 | NA | 0.087 |
| UHN | L061 | SQ | 1B | I | 8.414 | 0 | 8.414 | 0 | NA | 1.401 |
| UHN | L062 | SQ | 1A | I | 7.403 | 0 | 7.403 | 0 | NA | −2.038 |
| UHN | L066 | SQ | 2B | II | 7.479 | 0 | 7.181 | 1 | NA | −1.744 |
| UHN | L078 | SQ | 2B | II | 8.123 | 0 | 8.123 | 0 | NA | −0.484 |
| UHN | L083 | AD | 1B | I | 3.077 | 1 | 0.603 | 1 | NA | 1.108 |
| UHN | L086 | AD | 2A | II | 5.668 | 0 | 5.668 | 0 | NA | 0.046 |
| UHN | L093 | AD | 1B | I | 8.419 | 0 | 8.419 | 0 | NA | 0.057 |
| UHN | L095 | SQ | 3A | III+ | 5.159 | 0 | 5.159 | 0 | NA | −0.304 |
| UHN | L098 | AD | 2B | II | 1.578 | 1 | 1.005 | 1 | NA | 2.050 |
| UHN | L105 | AD | 1A | I | 4.666 | 1 | 1.444 | 1 | NA | 0.983 |
| UHN | L106 | SQ | 2B | II | 5.386 | 0 | 5.386 | 0 | NA | 0.174 |
| UHN | L112 | SQ | 2B | II | 5.082 | 0 | 5.082 | 0 | NA | −2.295 |
| UHN | L115 | SQ | 2B | II | 5.214 | 0 | 5.214 | 0 | NA | −0.025 |
| UHN | L116 | AD | 2B | II | 6.573 | 0 | 6.573 | 0 | NA | −0.447 |
| UHN | L120 | AD | 2A | II | 3.764 | 1 | 2.627 | 1 | NA | −0.707 |
| UHN | L123 | SQ | 2B | II | 6.244 | 0 | 6.244 | 0 | NA | −1.372 |
| UHN | L127 | SQ | 3A | III+ | 4.814 | 0 | 2.685 | 1 | NA | 0.960 |
| UHN | L133 | AD | 1A | I | 4.975 | 0 | 4.036 | 1 | NA | 1.160 |
| UHN | L148 | SQ | 1A | I | 4.885 | 0 | 4.885 | 0 | NA | −0.303 |
| UHN | L164 | AD | 1A | I | 6.181 | 0 | 6.181 | 0 | NA | 0.639 |
| UHN | L174 | AD | 1A | I | 4.088 | 1 | 0.975 | 1 | NA | 1.193 |
| UHN | L175 | AD | 1B | I | 5.699 | 0 | 5.699 | 0 | NA | 0.472 |
| UHN | L182 | SQ | 2A | II | 5.181 | 0 | 5.181 | 0 | NA | −1.234 |
| UHN | L191 | AD | 2A | II | 5.364 | 0 | 5.364 | 0 | NA | 0.555 |
| UHN | L195 | AD | 2B | II | 4.003 | 0 | 4.003 | 0 | NA | −0.713 |
| UHN | L197 | AD | 2A | II | 3.764 | NA | 3.764 | 0 | NA | −0.100 |
| UHN | L201 | SQ | 3A | III+ | 6.082 | 0 | 6.082 | 0 | NA | 1.965 |
| UHN | L212 | AD | 1B | I | 4.082 | 0 | 3.658 | 1 | NA | 0.095 |
| UHN | L214 | AD | 1B | I | 5.762 | 0 | 5.762 | 0 | NA | −0.159 |
| UHN | L218 | SQ | 3A | III+ | 4.153 | 0 | 4.153 | 0 | NA | −0.115 |
| UHN | L222 | AD | 3A | III+ | 3.260 | 0 | 2.112 | 1 | NA | 1.747 |
| UHN | P001 | AD | 1A | I | 7.005 | 0 | 6.252 | 1 | NA | 0.681 |
| UHN | P002 | AD | 2B | II | 3.858 | 1 | 3.025 | 1 | NA | −1.497 |
| UHN | P004 | SQ | 2B | II | 10.679 | 0 | 10.679 | 0 | NA | −0.495 |
| UHN | P006 | AD | 2B | II | 3.066 | 1 | 2.981 | 1 | NA | 1.549 |
| UHN | P009 | SQ | 2A | II | 6.074 | 0 | 6.074 | 0 | NA | 0.007 |
| UHN | P010 | AD | 1B | I | 6.967 | 0 | 6.967 | 0 | NA | −1.163 |
| UHN | P017 | SQ | 1B | I | 5.282 | 0 | 5.282 | 0 | NA | 0.033 |
| UHN | P020 | SQ | 3A | III+ | 1.485 | 1 | 1.359 | 1 | NA | 0.728 |
| UHN | P026 | AD | 1A | I | 5.389 | 0 | 5.389 | 0 | NA | −1.145 |
| UHN | P030 | AD | 1A | I | 4.984 | 0 | 4.984 | 0 | NA | −0.771 |
| UHN | P031 | AD | 1B | I | 0.622 | 1 | 0.444 | 1 | NA | 3.165 |
| UHN | P042 | SQ | 1B | I | 5.362 | 0 | 5.362 | 0 | NA | −1.323 |
| UHN | P043 | AD | 2A | II | 2.101 | 1 | 0.986 | 1 | NA | 0.338 |
| UHN | P046 | AD | 3A | III+ | 3.860 | 1 | 2.197 | 1 | NA | 1.945 |
| UHN | P080 | AD | 1B | I | 8.904 | 1 | 1.663 | 1 | NA | −0.239 |
| UHN | P081 | AD | 2B | II | 9.953 | 0 | 4.430 | 1 | NA | 1.370 |
| UHN | P085 | AD | 1B | I | 4.989 | 0 | 4.989 | 0 | NA | −0.179 |
| UHN | P086 | AD | 1A | I | 6.268 | 0 | 4.216 | 1 | NA | 1.360 |
| UHN | P089 | SQ | 1B | I | 3.992 | 0 | 3.992 | 0 | NA | 2.796 |
| UHN | P091 | SQ | 1B | I | 5.885 | 0 | 5.885 | 0 | NA | 1.175 |
| UHN | P092 | SQ | 1A | I | 6.219 | 0 | 6.219 | 0 | NA | −0.525 |
| UHN | P093 | SQ | 1B | I | 1.375 | 1 | 1.014 | 1 | NA | −0.626 |
| UHN | P100 | AD | 1A | I | 5.203 | 0 | 5.203 | 0 | NA | −0.061 |
| UHN | P106 | SQ | 2B | II | 3.156 | 1 | 1.068 | 1 | NA | 0.057 |
| UHN | P108 | AD | 3A | III+ | 1.353 | 1 | 0.852 | 1 | NA | 0.964 |
| UHN | P114 | AD | 3A | III+ | 0.918 | 1 | 0.110 | 1 | NA | 0.112 |
| UHN | P118 | AD | 3A | III+ | 8.447 | 0 | 8.447 | 0 | NA | 0.764 |
| UHN | P119 | AD | 2B | II | 3.422 | 0 | 3.422 | 0 | NA | 0.098 |
| UHN | P123 | AD | 1B | I | 0.685 | 1 | 0.575 | 1 | NA | 0.344 |
| UHN | P124 | AD | 3A | III+ | 3.173 | 1 | 3.132 | 1 | NA | −2.434 |
| UHN | P130 | AD | 1A | I | 8.921 | 0 | 8.921 | 0 | NA | −0.398 |
| UHN | P131 | AD | 1A | I | 3.877 | 1 | 3.230 | 1 | NA | −0.844 |
| UHN | P132 | AD | 1B | I | 2.208 | 1 | 1.258 | 1 | NA | 2.610 |
| UHN | P133 | SQ | 1B | I | 3.501 | 1 | 0.748 | 1 | NA | 0.000 |
| UHN | P135 | AD | 1B | I | 0.879 | 1 | 0.400 | 1 | NA | 0.232 |
| UHN | P136 | AD | 3A | III+ | 4.449 | 0 | 4.449 | 0 | NA | 0.619 |
| UHN | P140 | SQ | 1A | I | 3.874 | 0 | 3.874 | 0 | NA | −0.992 |
| UHN | P143 | AD | 1B | I | 5.490 | 0 | 5.490 | 0 | NA | 1.041 |
| UHN | P147 | AD | 1B | I | 2.063 | 1 | 1.767 | 1 | NA | 0.981 |
| UHN | P149 | SQ | 1A | I | 5.197 | 0 | 5.197 | 0 | NA | −1.224 |
| UHN | P152 | SQ | 1B | I | 0.953 | 1 | 0.953 | 1 | NA | 1.029 |
| UHN | P158 | AD | 1B | I | 2.411 | 1 | 1.416 | 1 | NA | 4.673 |
| UHN | P159 | SQ | 1B | I | 3.082 | 1 | 1.186 | 1 | NA | −0.272 |
| UHN | P163 | AD | 1B | I | 5.542 | 0 | 5.542 | 0 | NA | −0.702 |
| UHN | P164 | AD | 1A | I | 6.066 | 0 | 6.066 | 0 | NA | −0.201 |
| UHN | P166 | AD | 2B | II | 0.978 | 1 | 0.616 | 1 | NA | 1.905 |
| UHN | P167 | AD | 1B | I | 8.441 | 0 | 8.441 | 0 | NA | 1.485 |
| UHN | P168 | SQ | 1B | I | 3.775 | 0 | 1.570 | 1 | NA | 1.907 |
| UHN | P169 | AD | 1B | I | 0.586 | 1 | 0.381 | 1 | NA | 0.566 |
| UHN | P171 | AD | 2B | II | 1.666 | 1 | 1.534 | 1 | NA | 0.717 |
| UHN | P173 | AD | 1B | I | 3.575 | 0 | 3.575 | 0 | NA | −0.003 |
| UHN | P174 | SQ | 1B | I | 7.693 | 0 | 7.693 | 0 | NA | 0.150 |
| UHN | P177 | SQ | 1A | I | 2.663 | 0 | 1.211 | 1 | NA | −1.499 |
| UHN | P181 | SQ | 1B | I | 2.707 | 0 | 2.707 | 0 | NA | −1.376 |
| UHN | P185 | AD | 1A | I | 8.419 | 0 | 8.419 | 0 | NA | −1.095 |
| UHN | P186 | AD | 2B | II | 0.490 | 1 | 0.321 | 1 | NA | 0.412 |
| UHN | P188 | AD | 1A | I | 5.951 | 0 | 5.951 | 0 | NA | −0.952 |
| UHN | P189 | SQ | 2B | II | 2.937 | 0 | 2.463 | 1 | NA | −0.900 |
| UHN | P191 | AD | 1B | I | 7.400 | 1 | 5.537 | 1 | NA | 0.436 |
| UHN | P196 | SQ | 1A | I | 5.951 | 0 | 5.951 | 0 | NA | −1.065 |
| UHN | P201 | AD | 1A | I | 7.753 | 0 | 7.600 | 1 | NA | −1.518 |
| UHN | P204 | SQ | 1A | I | 4.395 | 0 | 4.395 | 0 | NA | −1.147 |
| UHN | P205 | AD | 1B | I | 7.784 | 0 | 7.784 | 0 | NA | 0.800 |
| UHN | P209 | SQ | 1A | I | 6.405 | 0 | 6.405 | 0 | NA | 1.129 |
| UHN | P210 | AD | 2B | II | 1.570 | 1 | 1.332 | 1 | NA | 1.772 |
| UHN | P214 | AD | 1B | I | 5.649 | 0 | 3.696 | 1 | NA | −0.527 |
| UHN | P215 | AD | 2B | II | 1.337 | 1 | 1.074 | 1 | NA | 2.324 |
| UHN | P218 | SQ | 1B | I | 2.241 | 1 | 1.997 | 1 | NA | 0.953 |
| UHN | P221 | AD | 1A | I | 5.049 | 0 | 5.049 | 0 | NA | 2.257 |
| UHN | P223 | AD | 1B | I | 4.455 | 1 | 2.170 | 1 | NA | −1.407 |
| UHN | P224 | AD | 1A | I | 6.888 | 0 | 6.888 | 0 | NA | −0.760 |
| UHN | P226 | AD | 1B | I | 1.921 | 0 | 1.921 | 0 | NA | −0.026 |
| UHN | P227 | AD | 3A | III+ | 3.099 | 0 | 3.099 | 0 | NA | −1.064 |
| UHN | P228 | SQ | 1A | I | 4.970 | 0 | 4.970 | 0 | NA | −0.733 |
| UHN | P230 | AD | 1B | I | 6.145 | 0 | 6.145 | 0 | NA | 0.389 |
| UHN | P238 | SQ | 1A | I | 0.778 | 0 | 0.778 | 0 | NA | −1.056 |
| UHN | P239 | SQ | 1A | I | 7.364 | 0 | 7.364 | 0 | NA | −1.095 |
| UHN | P240 | SQ | 1B | I | 7.647 | 0 | 7.647 | 0 | NA | 0.377 |
| UHN | P241 | AD | 1B | I | 5.800 | 0 | 5.800 | 0 | NA | −2.140 |
| UHN | P243 | SQ | 2B | II | 6.340 | 0 | 4.145 | 1 | NA | −0.943 |
| UHN | P245 | AD | 1A | I | 6.433 | 0 | 6.433 | 0 | NA | −0.021 |
| UHN | P248 | AD | 1A | I | 0.726 | 0 | 0.726 | 0 | NA | −1.575 |
| UHN | P250 | AD | 1B | I | 6.362 | 0 | 2.101 | 1 | NA | −1.487 |
| UHN | P253 | AD | 1A | I | 6.104 | 0 | 6.104 | 0 | NA | 2.219 |
| UHN | P254 | AD | 1B | I | 4.468 | 0 | 2.342 | 1 | NA | −2.930 |
| UHN | P257 | SQ | 1B | I | 2.488 | 0 | 2.488 | 0 | NA | −0.660 |
| UHN | P274 | AD | 1A | I | 4.307 | 0 | 4.307 | 0 | NA | −1.301 |
| UHN | P275 | AD | 1B | I | 6.564 | 0 | 6.564 | 0 | NA | 0.936 |
| UHN | P278 | SQ | 1B | I | 3.444 | 1 | 3.362 | 1 | NA | −1.630 |
| UHN | P284 | AD | 3A | III+ | 0.781 | 0 | 0.353 | 1 | NA | 0.015 |
| UHN | P287 | SQ | 1B | I | 4.748 | 0 | 4.748 | 0 | NA | −1.582 |
| UHN | P295 | SQ | 1B | I | 1.997 | 0 | 1.997 | 0 | NA | 2.093 |
| UHN | P302 | SQ | 1B | I | 4.997 | 0 | 4.997 | 0 | NA | −0.307 |
| UHN | P313 | SQ | 1B | I | 5.644 | 0 | 5.644 | 0 | NA | 0.251 |
| MI02 | AD10 | AD | 1A | I | 7.008 | 1 | NA | NA | NA | 0.022 |
| MI02 | AD2 | AD | 1A | I | 7.650 | 0 | NA | NA | 0 | −0.103 |
| MI02 | AD3 | AD | 1B | I | 7.808 | 0 | NA | NA | 0 | −0.503 |
| MI02 | AD5 | AD | 1B | I | 9.017 | 0 | NA | NA | 1 | −0.340 |
| MI02 | AD6 | AD | 1B | I | 2.883 | 1 | NA | NA | 1 | 0.221 |
| MI02 | AD7 | AD | 1A | I | 5.675 | 0 | NA | NA | 0 | −0.347 |
| MI02 | AD8 | AD | 1B | I | 2.850 | 0 | NA | NA | 0 | 0.030 |
| MI02 | L01 | AD | 1B | I | 3.917 | 0 | NA | NA | 0 | 0.046 |
| MI02 | L02 | AD | 1A | I | 3.258 | 0 | NA | NA | 0 | 0.234 |
| MI02 | L04 | AD | 1B | I | 3.817 | 1 | NA | NA | 0 | 0.264 |
| MI02 | L05 | AD | 1A | I | 9.217 | 0 | NA | NA | 0 | −0.276 |
| MI02 | L06 | AD | 1A | I | 7.658 | 0 | NA | NA | 1 | 0.314 |
| MI02 | L08 | AD | 1A | I | 8.992 | 0 | NA | NA | 1 | −0.147 |
| MI02 | L09 | AD | 1A | I | 8.225 | 0 | NA | NA | 1 | 0.001 |
| MI02 | L100 | AD | 1A | I | 3.650 | 0 | NA | NA | 0 | −0.001 |
| MI02 | L101 | AD | 1A | I | 3.333 | 0 | NA | NA | 0 | 0.027 |
| MI02 | L102 | AD | 1A | I | 3.333 | 0 | NA | NA | 0 | 1.059 |
| MI02 | L103 | AD | 1A | I | 2.567 | 0 | NA | NA | 0 | −0.079 |
| MI02 | L104 | AD | 1A | I | 2.033 | 0 | NA | NA | 0 | 0.364 |
| MI02 | L105 | AD | 1A | I | 2.358 | 0 | NA | NA | 1 | −0.235 |
| MI02 | L106 | AD | 1A | I | 2.108 | 0 | NA | NA | 0 | −0.405 |
| MI02 | L107 | AD | 1A | I | 1.083 | 0 | NA | NA | 1 | 0.372 |
| MI02 | L108 | AD | 1A | I | 1.625 | 0 | NA | NA | 1 | 0.370 |
| MI02 | L11 | AD | 1B | I | 2.892 | 1 | NA | NA | 1 | 0.211 |
| MI02 | L111 | AD | 1A | I | 0.125 | 0 | NA | NA | 1 | 0.156 |
| MI02 | L12 | AD | 1A | I | 7.100 | 0 | NA | NA | 0 | −0.124 |
| MI02 | L13 | AD | 1A | I | 6.625 | 1 | NA | NA | 1 | 0.003 |
| MI02 | L17 | AD | 1B | I | 6.975 | 0 | NA | NA | 1 | −0.171 |
| MI02 | L18 | AD | 1A | I | 4.017 | 0 | NA | NA | 0 | −0.269 |
| MI02 | L19 | AD | 3A | III+ | 0.800 | 1 | NA | NA | 1 | −0.056 |
| MI02 | L20 | AD | 1B | I | 1.658 | 1 | NA | NA | 0 | 0.141 |
| MI02 | L22 | AD | 1A | I | 1.042 | 0 | NA | NA | 0 | 0.011 |
| MI02 | L23 | AD | 3A | III+ | 1.258 | 0 | NA | NA | 1 | 0.177 |
| MI02 | L24 | AD | 1A | I | 0.133 | 0 | NA | NA | 0 | −0.053 |
| MI02 | L25 | AD | 1B | I | 1.208 | 0 | NA | NA | 1 | −0.013 |
| MI02 | L26 | AD | 1B | I | 1.475 | 0 | NA | NA | 1 | −0.219 |
| MI02 | L27 | AD | 1A | I | 1.758 | 0 | NA | NA | 0 | 0.200 |
| MI02 | L30 | AD | 1A | I | 1.683 | 0 | NA | NA | 0 | 0.059 |
| MI02 | L31 | AD | 1A | I | 2.100 | 0 | NA | NA | 0 | 0.149 |
| MI02 | L33 | AD | 3B | III+ | 2.450 | 0 | NA | NA | 0 | 0.251 |
| MI02 | L34 | AD | 3A | III+ | 1.242 | 1 | NA | NA | 0 | −0.362 |
| MI02 | L35 | AD | 3A | III+ | 2.350 | 1 | NA | NA | 1 | −0.406 |
| MI02 | L36 | AD | 3A | III+ | 0.600 | 1 | NA | NA | 1 | −0.004 |
| MI02 | L37 | AD | 3A | III+ | 0.217 | 1 | NA | NA | 1 | −0.510 |
| MI02 | L38 | AD | 3B | III+ | 0.833 | 0 | NA | NA | 1 | −0.127 |
| MI02 | L40 | AD | 3A | III+ | 1.675 | 1 | NA | NA | 0 | −0.140 |
| MI02 | L41 | AD | 1B | I | 0.700 | 1 | NA | NA | 1 | 0.030 |
| MI02 | L42 | AD | 1A | I | 5.283 | 0 | NA | NA | 0 | 0.184 |
| MI02 | L43 | AD | 1B | I | 6.542 | 0 | NA | NA | 0 | −0.644 |
| MI02 | L45 | AD | 1A | I | 2.467 | 1 | NA | NA | 1 | 0.114 |
| MI02 | L46 | AD | 1B | I | 6.867 | 0 | NA | NA | 1 | −0.200 |
| MI02 | L47 | AD | 1B | I | 5.042 | 0 | NA | NA | 1 | −0.100 |
| MI02 | L48 | AD | 1A | I | 6.483 | 0 | NA | NA | 0 | −0.039 |
| MI02 | L49 | AD | 1A | I | 5.892 | 0 | NA | NA | 1 | −0.285 |
| MI02 | L50 | AD | 1A | I | 1.583 | 1 | NA | NA | 1 | 0.083 |
| MI02 | L52 | AD | 1A | I | 5.450 | 0 | NA | NA | 0 | 0.392 |
| MI02 | L53 | AD | 3A | III+ | 1.383 | 1 | NA | NA | 0 | 0.324 |
| MI02 | L54 | AD | 3A | III+ | 0.333 | 1 | NA | NA | 1 | 1.008 |
| MI02 | L56 | AD | 1A | I | 5.150 | 0 | NA | NA | 0 | −0.064 |
| MI02 | L57 | AD | 1B | I | 4.567 | 0 | NA | NA | 1 | −0.083 |
| MI02 | L59 | AD | 3A | III+ | 4.550 | 0 | NA | NA | 1 | −0.020 |
| MI02 | L61 | AD | 1B | I | 1.717 | 1 | NA | NA | 0 | 0.238 |
| MI02 | L62 | AD | 3A | III+ | 4.367 | 0 | NA | NA | 0 | 0.015 |
| MI02 | L64 | AD | 1B | I | 4.008 | 0 | NA | NA | 0 | −0.051 |
| MI02 | L65 | AD | 1A | I | 4.408 | 0 | NA | NA | 0 | −0.074 |
| MI02 | L76 | AD | 1A | I | 7.308 | 0 | NA | NA | 1 | −0.108 |
| MI02 | L78 | AD | 1A | I | 3.042 | 0 | NA | NA | 1 | 0.083 |
| MI02 | L79 | AD | 1B | I | 0.725 | 1 | NA | NA | 0 | 0.185 |
| MI02 | L80 | AD | 1B | I | 0.842 | 1 | NA | NA | 1 | 0.539 |
| MI02 | L81 | AD | 1A | I | 3.000 | 0 | NA | NA | 0 | 1.636 |
| MI02 | L82 | AD | 1A | I | 2.842 | 0 | NA | NA | 0 | −0.199 |
| MI02 | L83 | AD | 1B | I | 2.550 | 0 | NA | NA | 0 | 0.143 |
| MI02 | L84 | AD | 1B | I | 2.683 | 0 | NA | NA | 0 | 0.148 |
| MI02 | L85 | AD | 1A | I | 2.233 | 0 | NA | NA | 1 | 0.118 |
| MI02 | L86 | AD | 1A | I | 0.842 | 0 | NA | NA | 0 | −0.068 |
| MI02 | L87 | AD | 1A | I | 0.867 | 0 | NA | NA | 0 | −0.297 |
| MI02 | L88 | AD | 1A | I | 0.692 | 0 | NA | NA | 1 | 0.561 |
| MI02 | L89 | AD | 3A | III+ | 1.017 | 0 | NA | NA | 1 | 0.892 |
| MI02 | L90 | AD | 1A | I | 0.483 | 1 | NA | NA | 0 | 1.021 |
| MI02 | L91 | AD | 3A | III+ | 0.508 | 0 | NA | NA | 0 | −0.231 |
| MI02 | L92 | AD | 3B | III+ | 0.708 | 0 | NA | NA | 0 | 0.411 |
| MI02 | L94 | AD | 3A | III+ | 0.200 | 1 | NA | NA | 0 | 0.187 |
| MI02 | L95 | AD | 3A | III+ | 0.450 | 1 | NA | NA | 1 | 0.183 |
| MI02 | L96 | AD | 3A | III+ | 1.767 | 1 | NA | NA | 1 | 0.201 |
| MI02 | L97 | AD | 1A | I | 0.408 | 0 | NA | NA | 1 | −0.405 |
| MI02 | L99 | AD | 1B | I | 0.375 | 0 | NA | NA | 1 | 0.525 |
| MIT | AD111 | AD | 1A | I | 6.033 | 0 | NA | NA | NA | 0.057 |
| MIT | AD114 | AD | 1A | I | 5.517 | 0 | NA | NA | NA | 0.326 |
| MIT | AD119 | AD | 1B | I | 6.383 | 0 | NA | NA | NA | 0.017 |
| MIT | AD123 | AD | 2B | II | 6.167 | 0 | NA | NA | NA | −0.014 |
| MIT | AD131 | AD | 1A | I | 6.333 | 0 | NA | NA | NA | −0.065 |
| MIT | AD136 | AD | 1B | I | 2.617 | 0 | NA | NA | NA | 0.098 |
| MIT | AD162 | AD | 1B | I | 3.475 | 0 | NA | NA | NA | −0.339 |
| MIT | AD167 | AD | 1B | I | 3.475 | 0 | NA | NA | NA | 0.082 |
| MIT | AD170 | AD | 1A | I | 6.533 | 0 | NA | NA | NA | −0.139 |
| MIT | AD172 | AD | 2B | II | 5.558 | 0 | NA | NA | NA | 0.605 |
| MIT | AD183 | AD | 1A | I | 3.517 | 0 | NA | NA | NA | −0.082 |
| MIT | AD186 | AD | 1A | I | 7.033 | 0 | NA | NA | NA | 0.436 |
| MIT | AD202 | AD | 4 | III+ | 4.917 | 0 | NA | NA | NA | 0.129 |
| MIT | AD203 | AD | 1A | I | 8.842 | 0 | NA | NA | NA | 0.395 |
| MIT | AD210 | AD | 1A | I | 4.942 | 0 | NA | NA | NA | 0.223 |
| MIT | AD212 | AD | 1B | I | 4.917 | 0 | NA | NA | NA | −0.417 |
| MIT | AD218 | AD | 2B | II | 5.150 | 0 | NA | NA | NA | 0.126 |
| MIT | AD221 | AD | 4 | III+ | 1.275 | 0 | NA | NA | NA | 0.279 |
| MIT | AD224 | AD | 1A | I | 4.542 | 0 | NA | NA | NA | 0.218 |
| MIT | AD226 | AD | 1A | I | 5.042 | 0 | NA | NA | NA | 0.358 |
| MIT | AD230 | AD | 1A | I | 4.725 | 0 | NA | NA | NA | −0.344 |
| MIT | AD232 | AD | 1A | I | 4.692 | 0 | NA | NA | NA | 0.092 |
| MIT | AD234 | AD | 2B | II | 2.842 | 0 | NA | NA | NA | 0.136 |
| MIT | AD239 | AD | 1B | I | 4.875 | 0 | NA | NA | NA | 0.08 |
| MIT | AD240 | AD | 1A | I | 3.625 | 0 | NA | NA | NA | 0.07 |
| MIT | AD243 | AD | 1A | I | 4.175 | 0 | NA | NA | NA | 0.039 |
| MIT | AD247 | AD | 1A | I | 5.925 | 0 | NA | NA | NA | −0.256 |
| MIT | AD250 | AD | 1A | I | 7.583 | 0 | NA | NA | NA | −0.116 |
| MIT | AD253 | AD | 4 | III+ | 4.933 | 0 | NA | NA | NA | 0.071 |
| MIT | AD255 | AD | 1B | I | 3.733 | 0 | NA | NA | NA | −0.403 |
| MIT | AD261 | AD | 1A | I | 4.800 | 0 | NA | NA | NA | −0.187 |
| MIT | AD267 | AD | 1B | I | 4.667 | 0 | NA | NA | NA | −0.527 |
| MIT | AD268 | AD | 1B | I | 4.175 | 0 | NA | NA | NA | −0.07 |
| MIT | AD294 | AD | 1A | I | 3.375 | 0 | NA | NA | NA | 0.018 |
| MIT | AD295 | AD | 1A | I | 3.792 | 0 | NA | NA | NA | −0.567 |
| MIT | AD305 | AD | 2A | II | 7.400 | 0 | NA | NA | NA | −0.243 |
| MIT | AD308 | AD | 1B | I | 6.583 | 0 | NA | NA | NA | −0.218 |
| MIT | AD311 | AD | 1B | I | 4.208 | 0 | NA | NA | NA | −0.096 |
| MIT | AD315 | AD | 2B | II | 4.725 | 0 | NA | NA | NA | 0.45 |
| MIT | AD317 | AD | 1B | I | 8.258 | 0 | NA | NA | NA | 0 |
| MIT | AD318 | AD | 1B | I | 6.917 | 0 | NA | NA | NA | 0.052 |
| MIT | AD320 | AD | 1A | I | 7.158 | 0 | NA | NA | NA | 0.374 |
| MIT | AD327 | AD | 1B | I | 6.825 | 0 | NA | NA | NA | 0.574 |
| MIT | AD331 | AD | 1A | I | 4.408 | 0 | NA | NA | NA | 0.015 |
| MIT | AD335 | AD | 2B | II | 3.908 | 0 | NA | NA | NA | −0.21 |
| MIT | AD337 | AD | 4 | III+ | 2.442 | 0 | NA | NA | NA | −0.098 |
| MIT | AD338 | AD | 1B | I | 6.283 | 0 | NA | NA | NA | 0.426 |
| MIT | AD346 | AD | 1A | I | 1.442 | 0 | NA | NA | NA | −0.321 |
| MIT | AD347 | AD | 1B | I | 0.042 | 0 | NA | NA | NA | −0.166 |
| MIT | AD353 | AD | 1B | I | 1.142 | 0 | NA | NA | NA | −0.308 |
| MIT | AD356 | AD | 1B | I | 4.100 | 0 | NA | NA | NA | −0.422 |
| MIT | AD367 | AD | 1B | I | 6.342 | 0 | NA | NA | NA | −0.204 |
| MIT | AD368 | AD | 1B | I | 5.217 | 0 | NA | NA | NA | −0.025 |
| MIT | AD379 | AD | 2B | II | 2.950 | 0 | NA | NA | NA | −0.197 |
| MIT | AD043 | AD | 4 | III+ | 1.175 | 1 | NA | NA | NA | 0.054 |
| MIT | AD115 | AD | 2B | II | 1.825 | 1 | NA | NA | NA | −0.004 |
| MIT | AD118 | AD | 1A | I | 4.133 | 1 | NA | NA | NA | −0.119 |
| MIT | AD120 | AD | 1B | I | 3.242 | 1 | NA | NA | NA | −0.108 |
| MIT | AD122 | AD | 2B | II | 2.825 | 1 | NA | NA | NA | 0.055 |
| MIT | AD127 | AD | 3A | III+ | 0.683 | 1 | NA | NA | NA | −0.005 |
| MIT | AD130 | AD | 2B | II | 0.592 | 1 | NA | NA | NA | 0.056 |
| MIT | AD157 | AD | 4 | III+ | 0.342 | 1 | NA | NA | NA | 0.103 |
| MIT | AD158 | AD | 1B | I | 3.392 | 1 | NA | NA | NA | 0.183 |
| MIT | AD159 | AD | 2B | II | 1.642 | 1 | NA | NA | NA | 0.569 |
| MIT | AD163 | AD | 2B | II | 7.225 | 1 | NA | NA | NA | 0.254 |
| MIT | AD164 | AD | 2B | II | 1.250 | 1 | NA | NA | NA | 0.192 |
| MIT | AD169 | AD | 1B | I | 1.667 | 1 | NA | NA | NA | 0.003 |
| MIT | AD173 | AD | 2B | II | 1.858 | 1 | NA | NA | NA | 0.355 |
| MIT | AD177 | AD | 3A | III+ | 0.233 | 1 | NA | NA | NA | −0.207 |
| MIT | AD178 | AD | 1A | I | 2.417 | 1 | NA | NA | NA | −0.029 |
| MIT | AD179 | AD | 1B | I | 2.025 | 1 | NA | NA | NA | 0.105 |
| MIT | AD185 | AD | 2B | II | 1.750 | 1 | NA | NA | NA | 0.279 |
| MIT | AD187 | AD | 1A | I | 7.192 | 1 | NA | NA | NA | 0.701 |
| MIT | AD188 | AD | 1B | I | 1.800 | 1 | NA | NA | NA | 0.225 |
| MIT | AD201 | AD | 3A | III+ | 1.025 | 1 | NA | NA | NA | 0.445 |
| MIT | AD207 | AD | 1B | I | 5.567 | 1 | NA | NA | NA | −0.051 |
| MIT | AD208 | AD | 4 | III+ | 1.250 | 1 | NA | NA | NA | 0.353 |
| MIT | AD213 | AD | 1A | I | 4.067 | 1 | NA | NA | NA | −0.278 |
| MIT | AD225 | AD | 1B | I | 0.217 | 1 | NA | NA | NA | −0.281 |
| MIT | AD228 | AD | 1B | I | 3.433 | 1 | NA | NA | NA | 0.13 |
| MIT | AD236 | AD | 1B | I | 1.183 | 1 | NA | NA | NA | −0.262 |
| MIT | AD238 | AD | 1A | I | 2.092 | 1 | NA | NA | NA | 0.356 |
| MIT | AD241 | AD | 4 | III+ | 2.225 | 1 | NA | NA | NA | −0.29 |
| MIT | AD249 | AD | 1A | I | 2.583 | 1 | NA | NA | NA | 0.093 |
| MIT | AD252 | AD | 1A | I | 1.375 | 1 | NA | NA | NA | 0.057 |
| MIT | AD258 | AD | 1B | I | 1.025 | 1 | NA | NA | NA | 0.158 |
| MIT | AD259 | AD | 2B | II | 1.708 | 1 | NA | NA | NA | −0.242 |
| MIT | AD260 | AD | 1B | I | 1.750 | 1 | NA | NA | NA | −0.296 |
| MIT | AD262 | AD | 3B | III+ | 1.383 | 1 | NA | NA | NA | −0.182 |
| MIT | AD266 | AD | 1A | I | 3.492 | 1 | NA | NA | NA | −0.307 |
| MIT | AD269 | AD | 1A | I | 4.025 | 1 | NA | NA | NA | −0.185 |
| MIT | AD275 | AD | 2B | II | 1.125 | 1 | NA | NA | NA | −0.04 |
| MIT | AD276 | AD | 3A | III+ | 0.375 | 1 | NA | NA | NA | 0.152 |
| MIT | AD277 | AD | 1A | I | 0.683 | 1 | NA | NA | NA | −0.202 |
| MIT | AD283 | AD | 1A | I | 3.933 | 1 | NA | NA | NA | −0.423 |
| MIT | AD285 | AD | 4 | III+ | 2.450 | 1 | NA | NA | NA | 0.119 |
| MIT | AD287 | AD | 3B | III+ | 0.617 | 1 | NA | NA | NA | −0.572 |
| MIT | AD296 | AD | 2A | II | 0.775 | 1 | NA | NA | NA | 0.044 |
| MIT | AD299 | AD | 1A | I | 3.158 | 1 | NA | NA | NA | 0.414 |
| MIT | AD301 | AD | 1B | I | 0.650 | 1 | NA | NA | NA | 0.406 |
| MIT | AD302 | AD | 3B | III+ | 4.817 | 1 | NA | NA | NA | 0.16 |
| MIT | AD304 | AD | 1B | I | 0.683 | 1 | NA | NA | NA | 0.328 |
| MIT | AD309 | AD | 1B | I | 3.133 | 1 | NA | NA | NA | 0.937 |
| MIT | AD313 | AD | 1A | I | 2.108 | 1 | NA | NA | NA | −0.046 |
| MIT | AD314 | AD | 4 | III+ | 2.467 | 1 | NA | NA | NA | −0.063 |
| MIT | AD323 | AD | 2B | II | 0.567 | 1 | NA | NA | NA | 0.041 |
| MIT | AD330 | AD | 2A | II | 0.608 | 1 | NA | NA | NA | 0.054 |
| MIT | AD332 | AD | I | I | 0.500 | 1 | NA | NA | NA | 0.406 |
| MIT | AD334 | AD | 4 | III+ | 0.008 | 1 | NA | NA | NA | 0.83 |
| MIT | AD336 | AD | 1B | I | 1.758 | 1 | NA | NA | NA | 0.182 |
| MIT | AD340 | AD | 4 | III+ | 1.558 | 1 | NA | NA | NA | −0.087 |
| MIT | AD341 | AD | 2B | II | 4.675 | 1 | NA | NA | NA | −0.091 |
| MIT | AD350 | AD | 4 | III+ | 2.925 | 1 | NA | NA | NA | 0.178 |
| MIT | AD351 | AD | 2A | II | 2.025 | 1 | NA | NA | NA | 1.707 |
| MIT | AD352 | AD | 4 | III+ | 0.350 | 1 | NA | NA | NA | −0.554 |
| MIT | AD361 | AD | 1B | I | 0.533 | 1 | NA | NA | NA | −0.173 |
| MIT | AD362 | AD | 1B | I | 5.958 | 1 | NA | NA | NA | 0.103 |
| MIT | AD363 | AD | 1B | I | 0.875 | 1 | NA | NA | NA | −0.409 |
| MIT | AD366 | AD | 3A | III+ | 0.783 | 1 | NA | NA | NA | 0.223 |
| MIT | AD370 | AD | 2B | II | 2.167 | 1 | NA | NA | NA | −0.391 |
| MIT | AD374 | AD | 1B | I | 0.733 | 1 | NA | NA | NA | −0.248 |
| MIT | AD375 | AD | 1B | I | 1.950 | 1 | NA | NA | NA | −0.192 |
| MIT | AD382 | AD | 3A | III+ | 2.508 | 1 | NA | NA | NA | 0.126 |
| MIT | AD383 | AD | 3A | III+ | 2.717 | 1 | NA | NA | NA | 0.225 |
| MIT | AD384 | AD | 4 | III+ | 1.267 | 1 | NA | NA | NA | −0.039 |
| Duke | 97-949 | NA | 1A | I | 4.819 | 0 | NA | NA | NA | −0.517 |
| Duke | 98-292 | NA | 1A | I | 5.503 | 0 | NA | NA | NA | −0.217 |
| Duke | 98-679 | NA | 1A | I | 4.986 | 0 | NA | NA | NA | 0.488 |
| Duke | 99-77 | NA | 2B | II | 1.164 | 0 | NA | NA | NA | 0.119 |
| Duke | 99-55 | NA | 3A | III+ | 0.967 | 1 | NA | NA | NA | 0.856 |
| Duke | 98-985 | NA | 1A | I | 2.900 | 0 | NA | NA | NA | 0.513 |
| Duke | 98-821 | NA | 3A | III+ | 2.973 | 0 | NA | NA | NA | 0.31 |
| Duke | 98-853 | NA | 1A | I | 0.431 | 0 | NA | NA | NA | 0.202 |
| Duke | 99-927 | NA | 1B | I | 2.925 | 0 | NA | NA | NA | −0.129 |
| Duke | 00-10 | NA | 2A | II | 1.206 | 1 | NA | NA | NA | 0.75 |
| Duke | 98-506 | NA | 2B | II | 5.925 | 0 | NA | NA | NA | −0.359 |
| Duke | 99-1033 | NA | 1A | I | 3.614 | 0 | NA | NA | NA | 0.653 |
| Duke | 98-320 | NA | 1B | I | 1.417 | 1 | NA | NA | NA | 0.14 |
| Duke | 98-711 | NA | 1B | I | 5.064 | 0 | NA | NA | NA | 0.129 |
| Duke | 98-401 | NA | 2A | II | 5.698 | 0 | NA | NA | NA | −0.525 |
| Duke | 96-3 | NA | 1B | I | 2.817 | 1 | NA | NA | NA | −0.296 |
| Duke | 97-1026 | NA | 2B | II | 1.092 | 1 | NA | NA | NA | −0.259 |
| Duke | 98-933 | NA | 1B | I | 2.342 | 1 | NA | NA | NA | 0.41 |
| Duke | 96-475 | NA | 1B | I | 7.273 | 0 | NA | NA | NA | 0.162 |
| Duke | 99-671 | NA | 1A | I | 4.878 | 0 | NA | NA | NA | −0.316 |
| Duke | 98-683 | NA | 1A | I | 2.798 | 1 | NA | NA | NA | 0.913 |
| Duke | 97-403 | NA | 1B | I | 0.723 | 1 | NA | NA | NA | 0.069 |
| Duke | 97-587 | NA | 1B | I | 3.273 | 1 | NA | NA | NA | 0.633 |
| Duke | 98-543 | NA | 1A | I | 2.008 | 0 | NA | NA | NA | −0.257 |
| Duke | 99-692 | NA | 1A | I | 2.658 | 1 | NA | NA | NA | −0.305 |
| Duke | 98-657 | NA | 1A | I | 3.300 | 1 | NA | NA | NA | 1.07 |
| Duke | 99-440 | NA | 1A | I | 2.933 | 0 | NA | NA | NA | 0.194 |
| Duke | 99-728 | NA | 1A | I | 4.053 | 0 | NA | NA | NA | 0.653 |
| Duke | 98-1146 | NA | 2B | II | 3.567 | 1 | NA | NA | NA | −0.437 |
| Duke | 98-771 | NA | 1A | I | 5.694 | 0 | NA | NA | NA | 0.499 |
| Duke | 98-1216 | NA | 2A | II | 1.411 | 1 | NA | NA | NA | 1.629 |
| Duke | 98-1014 | NA | 1B | I | 1.692 | 1 | NA | NA | NA | 0.195 |
| Duke | 99-830 | NA | 2A | II | 1.875 | 1 | NA | NA | NA | −0.295 |
| Duke | 00-11 | NA | 4 | III+ | 0.442 | 1 | NA | NA | NA | 0.056 |
| Duke | 98-152 | NA | 2B | II | 6.111 | 0 | NA | NA | NA | −0.251 |
| Duke | 98-1293 | NA | 1A | I | 4.950 | 0 | NA | NA | NA | −0.233 |
| Duke | 98-1296 | NA | 1A | I | 5.294 | 0 | NA | NA | NA | −0.163 |
| Duke | 98-375 | NA | 2B | II | 1.178 | 1 | NA | NA | NA | 0.314 |
| Duke | 98-967 | NA | 2B | II | 1.778 | 1 | NA | NA | NA | 0.065 |
| Duke | 99-1017 | NA | 1B | I | 4.525 | 0 | NA | NA | NA | −0.493 |
| Duke | 00-315 | NA | 1A | I | 3.767 | 0 | NA | NA | NA | 0.414 |
| Duke | 00-151 | NA | 1B | I | 0.528 | 1 | NA | NA | NA | −0.446 |
| Duke | 99-1067 | NA | 2B | II | 3.773 | 1 | NA | NA | NA | −0.245 |
| Duke | 99-301 | NA | 3A | III+ | 0.794 | 1 | NA | NA | NA | 1.045 |
| Duke | 99-137 | NA | 3A | III+ | 1.881 | 1 | NA | NA | NA | 0.33 |
| Duke | 98-1063 | NA | 2B | II | 1.598 | 1 | NA | NA | NA | −0.24 |
| Duke | 98-343 | NA | 1A | I | 4.125 | 0 | NA | NA | NA | −0.118 |
| Duke | 98-186 | NA | 1A | I | 4.119 | 1 | NA | NA | NA | −0.73 |
| Duke | 98-691 | NA | 1A | I | 0.408 | 1 | NA | NA | NA | 0.407 |
| Duke | 98-723 | NA | 1A | I | 1.039 | 1 | NA | NA | NA | −0.338 |
| Duke | 98-197 | NA | 1B | I | 5.906 | 0 | NA | NA | NA | 0 |
| Duke | 98-828 | NA | 1A | I | 3.650 | 0 | NA | NA | NA | −0.325 |
| Duke | 97-1027 | NA | 3A | III+ | 0.089 | 1 | NA | NA | NA | 0.081 |
| Duke | 00-327 | NA | 1B | I | 0.811 | 1 | NA | NA | NA | −0.621 |
| Duke | 98-438 | NA | 1B | I | 4.614 | 1 | NA | NA | NA | −0.3 |
| Duke | 98-1277 | NA | 1A | I | 4.661 | 0 | NA | NA | NA | −0.41 |
| Duke | 00-703 | NA | 1A | I | 3.553 | 0 | NA | NA | NA | −0.602 |
| Duke | 00-440 | NA | 1B | I | 2.406 | 1 | NA | NA | NA | 0.046 |
| Duke | 98-956 | NA | 1A | I | 4.956 | 0 | NA | NA | NA | −0.232 |
| Duke | 00-909 | NA | 1 | I | 0.931 | 1 | NA | NA | NA | −0.302 |
| Duke | 97-666 | NA | 1B | I | 4.273 | 1 | NA | NA | NA | 0.824 |
| Duke | 97-608 | NA | 1B | I | 6.764 | 0 | NA | NA | NA | −0.114 |
| Duke | 97-829 | NA | 2B | II | 1.028 | 1 | NA | NA | NA | −0.066 |
| Duke | 00-550 | NA | 1 | I | 2.786 | 0 | NA | NA | NA | −0.189 |
| Duke | 99-706 | NA | 1B | I | 4.936 | 0 | NA | NA | NA | −0.115 |
| Duke | 98-417 | NA | 1A | I | 2.267 | 1 | NA | NA | NA | 0.114 |
| Duke | 96-264 | NA | 1B | I | 6.911 | 0 | NA | NA | NA | −0.33 |
| Duke | 97-792 | NA | 2A | II | 6.219 | 0 | NA | NA | NA | −0.655 |
| Duke | 96-353 | NA | 1B | I | 2.364 | 1 | NA | NA | NA | 0.142 |
| Duke | 00-145 | NA | 1A | I | 4.269 | 0 | NA | NA | NA | 0.121 |
| Duke | 00-253 | NA | 1B | I | 1.028 | 0 | NA | NA | NA | −0.811 |
| Duke | 00-334 | NA | 1A | I | 3.125 | 0 | NA | NA | NA | 0.16 |
| Duke | 00-398 | NA | 1A | I | 2.428 | 1 | NA | NA | NA | 1.207 |
| Duke | 00-452 | NA | 1B | I | 2.817 | 1 | NA | NA | NA | 0.096 |
| Duke | 00-479 | NA | 1 | I | 0.158 | 1 | NA | NA | NA | 0.319 |
| Duke | 00-827 | NA | 1 | I | 1.106 | 1 | NA | NA | NA | −0.627 |
| Duke | 00-941 | NA | 1 | I | 2.028 | 1 | NA | NA | NA | 0.492 |
| Duke | 00-1059 | NA | 1 | I | 1.969 | 1 | NA | NA | NA | −0.037 |
| Duke | 00-1072 | NA | 2 | II | 3.473 | 0 | NA | NA | NA | −0.013 |
| Duke | 00-1082 | NA | 1 | I | 3.469 | 0 | NA | NA | NA | 1.474 |
| Duke | 01-181 | NA | 1A | I | 2.594 | 0 | NA | NA | NA | −0.344 |
| Duke | 01-189 | NA | 2B | II | 3.014 | 0 | NA | NA | NA | −0.166 |
| Duke | 01-236 | NA | 1B | I | 0.219 | 0 | NA | NA | NA | 0.028 |
| Duke | 01-331 | NA | 2B | II | 2.011 | 1 | NA | NA | NA | 1.609 |
| Duke | 01-646 | NA | 1B | I | 1.653 | 1 | NA | NA | NA | 0.411 |
| Duke | 01-284 | NA | 1A | I | 0.228 | 0 | NA | NA | NA | −0.01 |
| Duke | 01-369 | NA | 1B | I | 2.128 | 0 | NA | NA | NA | −0.875 |
| Duke | 01-424 | NA | 1A | I | 2.119 | 0 | NA | NA | NA | −0.111 |
| Duke | 01-534 | NA | 1B | I | 2.594 | 1 | NA | NA | NA | −0.228 |
| Duke | 01-139 | NA | 1A | I | 3.319 | 0 | NA | NA | NA | 0.683 |
| Duke | 97-930 | NA | 1B | I | 3.300 | 1 | NA | NA | NA | 0.173 |
| MI06 | LS-1 | SQ | 2B | II | 1.25 | 1 | NA | NA | NA | −0.099 |
| MI06 | LS-10 | SQ | 1B | I | 0.80833 | 1 | NA | NA | NA | −0.061 |
| MI06 | LS-100 | SQ | 1B | I | 1.69167 | 0 | NA | NA | NA | 0.442 |
| MI06 | LS-101 | SQ | 2B | II | 2.95 | 0 | NA | NA | NA | 0.066 |
| MI06 | LS-102 | SQ | 1B | I | 2.46667 | 0 | NA | NA | NA | −0.464 |
| MI06 | LS-103 | SQ | 2B | II | 2.36667 | 1 | NA | NA | NA | −0.655 |
| MI06 | LS-104 | SQ | 2B | II | 0.43333 | 1 | NA | NA | NA | 0.4 |
| MI06 | LS-105 | SQ | 2A | II | 2.40833 | 0 | NA | NA | NA | −2.473 |
| MI06 | LS-106 | SQ | 3A | III+ | 2.275 | 0 | NA | NA | NA | 0.309 |
| MI06 | LS-107 | SQ | 1B | I | 0.80833 | 1 | NA | NA | NA | 0.625 |
| MI06 | LS-108 | SQ | 1A | I | 2.41667 | 0 | NA | NA | NA | 0.679 |
| MI06 | LS-109 | SQ | 1B | I | 2.21667 | 0 | NA | NA | NA | −0.047 |
| MI06 | LS-111 | SQ | 1B | I | 1.38333 | 1 | NA | NA | NA | 0.152 |
| MI06 | LS-113 | SQ | 1B | I | 2.00833 | 0 | NA | NA | NA | 0.617 |
| MI06 | LS-114 | SQ | 1B | I | 1.95833 | 0 | NA | NA | NA | 0.824 |
| MI06 | LS-115 | SQ | 1B | I | 1.975 | 0 | NA | NA | NA | −0.351 |
| MI06 | LS-116 | SQ | 2B | II | 0.51667 | 0 | NA | NA | NA | 0.901 |
| MI06 | LS-117 | SQ | 1B | I | 4.98333 | 0 | NA | NA | NA | −0.369 |
| MI06 | LS-118 | SQ | 3A | III+ | 0.30833 | 1 | NA | NA | NA | 0.249 |
| MI06 | LS-119 | SQ | 2A | II | 1.70833 | 1 | NA | NA | NA | −0.273 |
| MI06 | LS-12 | SQ | 1B | I | 9.1 | 0 | NA | NA | NA | −0.112 |
| MI06 | LS-120 | SQ | 3B | III+ | 3.21667 | 0 | NA | NA | NA | 0.266 |
| MI06 | LS-121 | SQ | 2B | II | 2.89167 | 0 | NA | NA | NA | 0.301 |
| MI06 | LS-122 | SQ | 1A | I | 0.86667 | 1 | NA | NA | NA | 0.172 |
| MI06 | LS-123 | SQ | 1A | I | 2.60833 | 0 | NA | NA | NA | 0.485 |
| MI06 | LS-124 | SQ | 1B | I | 2.64167 | 0 | NA | NA | NA | 0.134 |
| MI06 | LS-125 | SQ | 1B | I | 0.78333 | 1 | NA | NA | NA | 0.044 |
| MI06 | LS-126 | SQ | 3A | III+ | 2.375 | 1 | NA | NA | NA | −0.05 |
| MI06 | LS-127 | SQ | 3A | III+ | 0.61667 | 1 | NA | NA | NA | 0.204 |
| MI06 | LS-128 | SQ | 1A | I | 1.35 | 1 | NA | NA | NA | −0.262 |
| MI06 | LS-129 | SQ | 1B | I | 2.85 | 0 | NA | NA | NA | −0.183 |
| MI06 | LS-13 | SQ | 1B | I | 0.80833 | 1 | NA | NA | NA | −0.011 |
| MI06 | LS-130 | SQ | 2B | II | 3.25 | 0 | NA | NA | NA | −0.036 |
| MI06 | LS-131 | SQ | 1A | I | 1.99167 | 0 | NA | NA | NA | 1.04 |
| MI06 | LS-132 | SQ | 3B | III+ | 0.71667 | 1 | NA | NA | NA | 0.802 |
| MI06 | LS-133 | SQ | 2B | II | 2.51667 | 0 | NA | NA | NA | −0.187 |
| MI06 | LS-134 | SQ | 1A | I | 0.675 | 1 | NA | NA | NA | −0.216 |
| MI06 | LS-135 | SQ | 2B | II | 1.55833 | 0 | NA | NA | NA | 0.14 |
| MI06 | LS-136 | SQ | 2B | II | 6.50833 | 0 | NA | NA | NA | −0.611 |
| MI06 | LS-138 | SQ | 2B | II | 9.44167 | 0 | NA | NA | NA | 0.142 |
| MI06 | LS-139 | SQ | 1A | I | 2.4 | 1 | NA | NA | NA | 0.009 |
| MI06 | LS-14 | SQ | 1B | I | 1.68333 | 1 | NA | NA | NA | 0.525 |
| MI06 | LS-140 | SQ | 1B | I | 3.8 | 1 | NA | NA | NA | 0.033 |
| MI06 | LS-15 | SQ | 2B | II | 3.1 | 1 | NA | NA | NA | 0.208 |
| MI06 | LS-16 | SQ | 1B | I | 9.95833 | 1 | NA | NA | NA | −0.52 |
| MI06 | LS-17 | SQ | 3A | III+ | 10.0167 | 0 | NA | NA | NA | −0.332 |
| MI06 | LS-18 | SQ | 3A | III+ | 10.075 | 0 | NA | NA | NA | −1.819 |
| MI06 | LS-19 | SQ | 3A | III+ | 0.4 | 1 | NA | NA | NA | −0.18 |
| MI06 | LS-2 | SQ | 1B | I | 11.975 | 0 | NA | NA | NA | −0.047 |
| MI06 | LS-20 | SQ | 2A | II | 10.6333 | 0 | NA | NA | NA | −0.294 |
| MI06 | LS-21 | SQ | 3B | III+ | 8.46667 | 1 | NA | NA | NA | −0.1 |
| MI06 | LS-22 | SQ | 3B | III+ | 0.49167 | 1 | NA | NA | NA | −0.071 |
| MI06 | LS-23 | SQ | 3A | III+ | 8.65 | 0 | NA | NA | NA | 0.873 |
| MI06 | LS-24 | SQ | 3B | III+ | 9.275 | 0 | NA | NA | NA | −0.156 |
| MI06 | LS-25 | SQ | 1A | I | 5.73333 | 0 | NA | NA | NA | −0.074 |
| MI06 | LS-26 | SQ | 1B | I | 5.71667 | 1 | NA | NA | NA | 0.033 |
| MI06 | LS-27 | SQ | 1B | I | 0.50833 | 1 | NA | NA | NA | 0.134 |
| MI06 | LS-28 | SQ | 1A | I | 0.975 | 1 | NA | NA | NA | −0.261 |
| MI06 | LS-29 | SQ | 1A | I | 5.19167 | 1 | NA | NA | NA | 0.139 |
| MI06 | LS-30 | SQ | 1B | I | 7.80833 | 0 | NA | NA | NA | −0.529 |
| MI06 | LS-31 | SQ | 1A | I | 10.775 | 1 | NA | NA | NA | 0.29 |
| MI06 | LS-32 | SQ | 1B | I | 5.34167 | 1 | NA | NA | NA | −0.345 |
| MI06 | LS-33 | SQ | 3A | III+ | 0.675 | 1 | NA | NA | NA | 0.312 |
| MI06 | LS-34 | SQ | 3A | III+ | 5.85833 | 1 | NA | NA | NA | −0.081 |
| MI06 | LS-35 | SQ | 1B | I | 4.05833 | 0 | NA | NA | NA | −0.068 |
| MI06 | LS-36 | SQ | 1B | I | 3.28333 | 1 | NA | NA | NA | 0.324 |
| MI06 | LS-37 | SQ | 1B | I | 7.525 | 0 | NA | NA | NA | 0.219 |
| MI06 | LS-38 | SQ | 1B | I | 3.89167 | 0 | NA | NA | NA | 0.075 |
| MI06 | LS-39 | SQ | 3B | III+ | 0.33333 | 1 | NA | NA | NA | −0.081 |
| MI06 | LS-40 | SQ | 1A | I | 5.725 | 1 | NA | NA | NA | −0.084 |
| MI06 | LS-41 | SQ | 1A | I | 6.16667 | 0 | NA | NA | NA | 0.339 |
| MI06 | LS-42 | SQ | 1A | I | 2.59167 | 1 | NA | NA | NA | −0.023 |
| MI06 | LS-43 | SQ | 1A | I | 6.475 | 0 | NA | NA | NA | −0.395 |
| MI06 | LS-44 | SQ | 1B | I | 0.85833 | 1 | NA | NA | NA | 0.067 |
| MI06 | LS-45 | SQ | 1B | I | 2.25 | 1 | NA | NA | NA | −0.218 |
| MI06 | LS-46 | SQ | 1B | I | 5.39167 | 0 | NA | NA | NA | 0.048 |
| MI06 | LS-47 | SQ | 1A | I | 2.04167 | 1 | NA | NA | NA | 0.012 |
| MI06 | LS-48 | SQ | 1B | I | 5.275 | 0 | NA | NA | NA | −0.147 |
| MI06 | LS-49 | SQ | 1B | I | 4.05 | 1 | NA | NA | NA | −0.285 |
| MI06 | LS-5 | SQ | 3A | III+ | 0.73333 | 1 | NA | NA | NA | 0.21 |
| MI06 | LS-50 | SQ | 1A | I | 4.775 | 0 | NA | NA | NA | 0.154 |
| MI06 | LS-51 | SQ | 1A | I | 5.23333 | 0 | NA | NA | NA | −0.763 |
| MI06 | LS-52 | SQ | 1B | I | 0.85 | 1 | NA | NA | NA | 0.693 |
| MI06 | LS-53 | SQ | 1A | I | 4.5 | 0 | NA | NA | NA | 0.146 |
| MI06 | LS-54 | SQ | 1B | I | 5.2 | 0 | NA | NA | NA | 0.089 |
| MI06 | LS-55 | SQ | 3A | III+ | 1.925 | 1 | NA | NA | NA | 0.799 |
| MI06 | LS-56 | SQ | 2B | II | 2.24167 | 1 | NA | NA | NA | −0.542 |
| MI06 | LS-57 | SQ | 1B | I | 4.51667 | 0 | NA | NA | NA | 0.671 |
| MI06 | LS-58 | SQ | 1B | I | 1.36667 | 1 | NA | NA | NA | 1.243 |
| MI06 | LS-59 | SQ | 2B | II | 8.775 | 0 | NA | NA | NA | 0.272 |
| MI06 | LS-6 | SQ | 1B | I | 1.00833 | 1 | NA | NA | NA | −0.019 |
| MI06 | LS-60 | SQ | 3A | III+ | 7.95833 | 1 | NA | NA | NA | 0.234 |
| MI06 | LS-61 | SQ | 2B | II | 11.8583 | 0 | NA | NA | NA | 0.931 |
| MI06 | LS-62 | SQ | 3A | III+ | 9.54167 | 1 | NA | NA | NA | −0.554 |
| MI06 | LS-63 | SQ | 1B | I | 10.0833 | 0 | NA | NA | NA | −0.614 |
| MI06 | LS-64 | SQ | 2B | II | 5.18333 | 1 | NA | NA | NA | 0.647 |
| MI06 | LS-65 | SQ | 2B | II | 4.96667 | 0 | NA | NA | NA | 0.006 |
| MI06 | LS-66 | SQ | 2B | II | 7.875 | 1 | NA | NA | NA | −0.216 |
| MI06 | LS-67 | SQ | 2B | II | 5.34167 | 1 | NA | NA | NA | −0.789 |
| MI06 | LS-68 | SQ | 2B | II | 10.9583 | 0 | NA | NA | NA | −0.024 |
| MI06 | LS-69 | SQ | 1B | I | 6.575 | 1 | NA | NA | NA | 0.279 |
| MI06 | LS-70 | SQ | 1A | I | 6.74167 | 1 | NA | NA | NA | 0.071 |
| MI06 | LS-71 | SQ | 2B | II | 6.50833 | 0 | NA | NA | NA | −1.115 |
| MI06 | LS-72 | SQ | 1B | I | 0.61667 | 1 | NA | NA | NA | −0.385 |
| MI06 | LS-73 | SQ | 2B | II | 1.825 | 0 | NA | NA | NA | 0.23 |
| MI06 | LS-74 | SQ | 1B | I | 2.75833 | 1 | NA | NA | NA | −0.064 |
| MI06 | LS-75 | SQ | 2B | II | 4.21667 | 0 | NA | NA | NA | −0.063 |
| MI06 | LS-77 | SQ | 3A | III+ | 0.3 | 1 | NA | NA | NA | 0.529 |
| MI06 | LS-78 | SQ | 3A | III+ | 4.525 | 1 | NA | NA | NA | −0.498 |
| MI06 | LS-79 | SQ | 2B | II | 0.9 | 1 | NA | NA | NA | 0.421 |
| MI06 | LS-8 | SQ | 1B | I | 11.3417 | 0 | NA | NA | NA | −0.344 |
| MI06 | LS-80 | SQ | 2B | II | 0.33333 | 1 | NA | NA | NA | −0.545 |
| MI06 | LS-81 | SQ | 1B | I | 4.29167 | 0 | NA | NA | NA | 0.165 |
| MI06 | LS-82 | SQ | 1A | I | 4.11667 | 0 | NA | NA | NA | 0.571 |
| MI06 | LS-83 | SQ | 2A | II | 2.89167 | 1 | NA | NA | NA | 0.277 |
| MI06 | LS-85 | SQ | 1A | I | 3.95 | 0 | NA | NA | NA | −0.231 |
| MI06 | LS-86 | SQ | 1B | I | 3.71667 | 0 | NA | NA | NA | 0.059 |
| MI06 | LS-87 | SQ | 2A | II | 0.18333 | 1 | NA | NA | NA | −0.222 |
| MI06 | LS-88 | SQ | 2B | II | 0.69167 | 1 | NA | NA | NA | −1.936 |
| MI06 | LS-89 | SQ | 1A | I | 3.65833 | 0 | NA | NA | NA | 0.448 |
| MI06 | LS-9 | SQ | 2B | II | 0.275 | 1 | NA | NA | NA | −0.489 |
| MI06 | LS-90 | SQ | 1A | I | 3.675 | 0 | NA | NA | NA | −0.006 |
| MI06 | LS-91 | SQ | 2B | II | 3.41667 | 0 | NA | NA | NA | −0.028 |
| MI06 | LS-92 | SQ | 1A | I | 2.84167 | 0 | NA | NA | NA | −0.748 |
| MI06 | LS-94 | SQ | 3A | III+ | 1.15 | 1 | NA | NA | NA | −0.687 |
| MI06 | LS-95 | SQ | 1B | I | 0.88333 | 1 | NA | NA | NA | 0.504 |
| MI06 | LS-96 | SQ | 1A | I | 2.16667 | 0 | NA | NA | NA | 0.225 |
| MI06 | LS-97 | SQ | 2A | II | 0.64167 | 1 | NA | NA | NA | 0.309 |
| MI06 | LS-98 | SQ | 1B | I | 1.075 | 1 | NA | NA | NA | −1.708 |
| MI06 | LS-99 | SQ | 1A | I | 2.93333 | 0 | NA | NA | NA | −0.183 |
| AD1 | Sample_A1 | AD | 1B | I | 10.4008 | 0 | NA | NA | NA | −0.078 |
| AD1 | Sample_A2 | AD | 1A | I | 10.3433 | 1 | NA | NA | NA | 0.181 |
| AD1 | Sample_A3 | AD | 1A | I | 14.0725 | 0 | NA | NA | NA | −0.145 |
| AD1 | Sample_A4 | AD | 1A | I | 15.3425 | 0 | NA | NA | NA | −0.054 |
| AD1 | Sample_A5 | AD | 1A | I | 12.9058 | 0 | NA | NA | NA | −0.091 |
| AD1 | Sample_A6 | AD | 1B | I | 12.3617 | 0 | NA | NA | NA | 0.357 |
| AD1 | Sample_A8 | AD | 1B | I | 11.0775 | 0 | NA | NA | NA | 0.189 |
| AD1 | Sample_A9 | AD | 1B | I | 6.94583 | 1 | NA | NA | NA | −0.235 |
| AD1 | Sample_A10 | AD | 1A | I | 5.76833 | 0 | NA | NA | NA | 0.079 |
| AD1 | Sample_A11 | AD | 1A | I | 9.47333 | 0 | NA | NA | NA | 0.043 |
| AD1 | Sample_A12 | AD | 1A | I | 7.71 | 0 | NA | NA | NA | −0.196 |
| AD1 | Sample_A13 | AD | 1B | I | 5.87 | 0 | NA | NA | NA | 0.083 |
| AD1 | Sample_A14 | AD | 1A | I | 5.88083 | 0 | NA | NA | NA | −0.178 |
| AD1 | Sample_A15 | AD | 1B | I | 5.81833 | 0 | NA | NA | NA | 0.214 |
| AD1 | Sample_A16 | AD | 1A | I | 5.54667 | 0 | NA | NA | NA | −0.046 |
| AD1 | Sample_A17 | AD | 1A | I | 5.60417 | 0 | NA | NA | NA | −0.17 |
| AD1 | Sample_A18 | AD | 1A | I | 5.87583 | 0 | NA | NA | NA | 0.003 |
| AD1 | Sample_A19 | AD | 1B | I | 4.82417 | 0 | NA | NA | NA | 0.352 |
| AD1 | Sample_A20 | AD | 1B | I | 4.67583 | 1 | NA | NA | NA | 0.311 |
| AD1 | Sample_A21 | AD | 1A | I | 4.53917 | 0 | NA | NA | NA | −0.181 |
| AD1 | Sample_A22 | AD | 1B | I | 4.42167 | 0 | NA | NA | NA | 0 |
| AD1 | Sample_A23 | AD | 1B | I | 4.2325 | 0 | NA | NA | NA | 0.022 |
| AD1 | Sample_A24 | AD | 1A | I | 4.45 | 0 | NA | NA | NA | 0.032 |
| AD1 | Sample_A25 | AD | 1B | I | 3.83583 | 0 | NA | NA | NA | 0.352 |
| AD1 | Sample_A26 | AD | 1B | I | 3.69917 | 0 | NA | NA | NA | −0.029 |
| AD1 | Sample_A27 | AD | 1B | I | 13.67 | 0 | NA | NA | NA | 0.172 |
| AD1 | Sample_A28 | AD | 1B | I | 0.5475 | 1 | NA | NA | NA | NA |
| AD1 | Sample_A29 | AD | 1B | I | 2.02833 | 1 | NA | NA | NA | −0.149 |
| AD1 | Sample_A30 | AD | 1B | I | 1.81833 | 1 | NA | NA | NA | 0.058 |
| AD1 | Sample_A31 | AD | 1B | I | 4.55583 | 1 | NA | NA | NA | 0.023 |
| AD1 | Sample_A32 | AD | 1B | I | 0.66 | 1 | NA | NA | NA | −6E−04 |
| AD1 | Sample_A33 | AD | 2B | II | 2.05333 | 1 | NA | NA | NA | −0.126 |
| AD1 | Sample_A34 | AD | 1B | I | 0.35083 | 1 | NA | NA | NA | −0.205 |
| AD1 | Sample_A35 | AD | 1A | I | 2.52667 | 1 | NA | NA | NA | −0.11 |
| AD1 | Sample_A36 | AD | 1A | I | 1.125 | 1 | NA | NA | NA | 0.25 |
| AD1 | Sample_A37 | AD | 1B | I | 1.18583 | 1 | NA | NA | NA | −0.499 |
| AD1 | Sample_A38 | AD | 1B | I | 1.16917 | 1 | NA | NA | NA | 0.134 |
| AD1 | Sample_A39 | AD | 1B | I | 1.28667 | 1 | NA | NA | NA | 0.131 |
| AD1 | Sample_A40 | AD | 1B | I | 5.36333 | 0 | NA | NA | NA | −0.018 |
| AD1 | Sample_A41 | AD | 1B | I | 2.20667 | 1 | NA | NA | NA | 0.103 |
| AD1 | Sample_A42 | AD | 1B | I | 2.18167 | 1 | NA | NA | NA | −0.242 |
| AD1 | Sample_A43 | AD | 1A | I | 2.06167 | 1 | NA | NA | NA | −0.003 |
| AD1 | Sample_A44 | AD | 1B | I | 2.15167 | 1 | NA | NA | NA | −0.292 |
| AD1 | Sample_A45 | AD | 2B | II | 0.68417 | 1 | NA | NA | NA | 0.032 |
| AD1 | Sample_A46 | AD | 1B | I | 1.07333 | 1 | NA | NA | NA | −0.151 |
| AD1 | Sample_A47 | AD | 1B | I | 2.25833 | 1 | NA | NA | NA | −0.038 |
| AD1 | Sample_A48 | AD | 1B | I | 0.9525 | 1 | NA | NA | NA | 0.374 |
| AD1 | Sample_A49 | AD | 1B | I | 2.795 | 0 | NA | NA | NA | 0.048 |
| SQ2 | Sample_N1 | SQ | 1B | I | 5.0925 | 1 | NA | NA | NA | 0.106 |
| SQ2 | Sample_N2 | SQ | 1A | I | 12.8025 | 1 | NA | NA | NA | 0.042 |
| SQ2 | Sample_N3 | SQ | 1B | I | 9.34667 | 1 | NA | NA | NA | −0.243 |
| SQ2 | Sample_N4 | SQ | 1A | I | 15.8958 | 0 | NA | NA | NA | 0 |
| SQ2 | Sample_N5 | SQ | 1B | I | 10.4967 | 1 | NA | NA | NA | 0.121 |
| SQ2 | Sample_N6 | SQ | 1B | I | 10.6667 | 1 | NA | NA | NA | −0.032 |
| SQ2 | Sample_N7 | SQ | 1B | I | 10.8608 | 0 | NA | NA | NA | 0.121 |
| SQ2 | Sample_N8 | SQ | 1B | I | 6.105 | 0 | NA | NA | NA | 0.003 |
| SQ2 | Sample_N9 | SQ | 1B | I | 10.3733 | 0 | NA | NA | NA | −0.011 |
| SQ2 | Sample_N10 | SQ | 3B | III+ | 8.06333 | 0 | NA | NA | NA | −0.004 |
| SQ2 | Sample_N11 | SQ | 1B | I | 6.68583 | 0 | NA | NA | NA | 0.006 |
| SQ2 | Sample_N12 | SQ | 2B | II | 10.0342 | 0 | NA | NA | NA | 0.037 |
| SQ2 | Sample_N13 | SQ | 1B | I | 8.345 | 1 | NA | NA | NA | −0.144 |
| SQ2 | Sample_N14 | SQ | 1A | I | 8.29833 | 0 | NA | NA | NA | 0.14 |
| SQ2 | Sample_N15 | SQ | 1A | I | 6.83917 | 0 | NA | NA | NA | 0.19 |
| SQ2 | Sample_N16 | SQ | 1B | I | 7.745 | 0 | NA | NA | NA | 0.185 |
| SQ2 | Sample_N17 | SQ | 1B | I | 13.1283 | 0 | NA | NA | NA | 0.203 |
| SQ2 | Sample_N18 | SQ | 1A | I | 8.23833 | 0 | NA | NA | NA | 0.182 |
| SQ2 | Sample_N19 | SQ | 1B | I | 7.67167 | 0 | NA | NA | NA | −0.008 |
| SQ2 | Sample_N20 | SQ | 1B | I | 3.8825 | 1 | NA | NA | NA | −0.175 |
| SQ2 | Sample_N21 | SQ | 1B | I | 5.8375 | 0 | NA | NA | NA | 0.104 |
| SQ2 | Sample_N22 | SQ | 1A | I | 5.02417 | 0 | NA | NA | NA | −0.115 |
| SQ2 | Sample_N23 | SQ | 3B | III+ | 5.24833 | 0 | NA | NA | NA | 0.299 |
| SQ2 | Sample_N24 | SQ | 1B | I | 5.38333 | 0 | NA | NA | NA | −0.1 |
| SQ2 | Sample_N25 | SQ | 1B | I | 3.89583 | 0 | NA | NA | NA | 0.13 |
| SQ2 | Sample_N26 | SQ | 2A | II | 13.4542 | 0 | NA | NA | NA | −0.035 |
| SQ2 | Sample_N27 | SQ | 3A | III+ | 5.125 | 1 | NA | NA | NA | 0.077 |
| SQ2 | Sample_N28 | SQ | 2B | II | 5.65083 | 0 | NA | NA | NA | 0.14 |
| SQ2 | Sample_N29 | SQ | 2B | II | 6.14917 | 0 | NA | NA | NA | 0.125 |
| SQ2 | Sample_N30 | SQ | 2B | II | 5.7275 | 0 | NA | NA | NA | 0.023 |
| SQ2 | Sample_N31 | SQ | 2B | II | 5.2125 | 0 | NA | NA | NA | 0.046 |
| SQ2 | Sample_N32 | SQ | 3A | III+ | 4.7 | 0 | NA | NA | NA | 0.21 |
| SQ2 | Sample_R1 | SQ | 2B | II | 0.43 | 1 | NA | NA | NA | −0.039 |
| SQ2 | Sample_R2 | SQ | 1B | I | 1.48417 | 1 | NA | NA | NA | 0.214 |
| SQ2 | Sample_R3 | SQ | 1A | I | 4.0275 | 1 | NA | NA | NA | 0.103 |
| SQ2 | Sample_R4 | SQ | 1B | I | 1.61 | 1 | NA | NA | NA | −0.054 |
| SQ2 | Sample_R5 | SQ | 1A | I | 1.6725 | 1 | NA | NA | NA | −0.098 |
| SQ2 | Sample_R6 | SQ | 1B | I | 2.55417 | 1 | NA | NA | NA | −0.155 |
| SQ2 | Sample_R7 | SQ | 1B | I | 1.31667 | 1 | NA | NA | NA | 0.181 |
| SQ2 | Sample_R8 | SQ | 1B | I | 0.79917 | 1 | NA | NA | NA | 0.076 |
| SQ2 | Sample_R9 | SQ | 2B | II | 0.76083 | 1 | NA | NA | NA | −0.017 |
| SQ2 | Sample_R10 | SQ | 2B | II | 2.0175 | 1 | NA | NA | NA | −0.186 |
| SQ2 | Sample_R11 | SQ | 3A | III+ | 2.2125 | 1 | NA | NA | NA | −0.042 |
| SQ2 | Sample_R12 | SQ | 2B | II | 1.85667 | 1 | NA | NA | NA | 0.237 |
| SQ2 | Sample_R13 | SQ | 2B | II | 1.38833 | 1 | NA | NA | NA | −0.213 |
| SQ2 | Sample_R14 | SQ | 2B | II | 2.46167 | 1 | NA | NA | NA | 0.231 |
| SQ2 | Sample_R15 | SQ | 2B | II | 0.59417 | 1 | NA | NA | NA | −0.038 |
| SQ2 | Sample_R16 | SQ | 2B | II | 0.5425 | 1 | NA | NA | NA | −0.172 |
| SQ2 | Sample_R17 | SQ | 2B | II | 1.73 | 1 | NA | NA | NA | −0.033 |
| SQ2 | Sample_R18 | SQ | 3A | III+ | 1.845 | 1 | NA | NA | NA | −0.06 |
| SQ2 | Sample_R19 | SQ | 3A | III+ | 1.6675 | 1 | NA | NA | NA | −0.034 |
| SQ2 | Sample_S1 | SQ | 2B | II | 1.59583 | 1 | NA | NA | NA | −0.06 |
| SQ2 | Sample_S2 | SQ | 2B | II | 5.1775 | 0 | NA | NA | NA | −0.139 |
| SQ2 | Sample_S3 | SQ | 2B | II | 0.63833 | 1 | NA | NA | NA | 0.201 |
| SQ2 | Sample_S4 | SQ | 2B | II | 2.565 | 1 | NA | NA | NA | −0.108 |
| SQ2 | Sample_S5 | SQ | 2B | II | 2.765 | 1 | NA | NA | NA | −0.135 |
| SQ2 | Sample_S6 | SQ | 4 | III+ | 1.39667 | 1 | NA | NA | NA | −0.031 |
| SQ2 | Sample_S7 | SQ | 2A | II | 2.57333 | 1 | NA | NA | NA | 0.083 |
| SQ2 | Sample_S8 | SQ | 1B | I | 1.36083 | 1 | NA | NA | NA | −0.355 |
| LuMayo | 40430 | SQ | 1B | I | 2.27242 | 1 | NA | NA | NA | −0.116 |
| LuMayo | 41923 | SQ | 1A | I | 5.02122 | 0 | NA | NA | NA | −0.536 |
| LuMayo | 41932 | SQ | 1B | I | 4.3833 | 0 | NA | NA | NA | 1.377 |
| LuMayo | 42081 | SQ | 1B | I | 5.40726 | 0 | NA | NA | NA | 0.195 |
| LuMayo | 42613 | SQ | 1B | I | 1.77413 | 1 | NA | NA | NA | −0.024 |
| LuMayo | 42616 | SQ | 1A | I | 5.37714 | 0 | NA | NA | NA | 0.039 |
| LuMayo | 44656 | SQ | 1B | I | 4.83504 | 0 | NA | NA | NA | −0.23 |
| LuMayo | 44661 | SQ | 1B | I | 0.74743 | 1 | NA | NA | NA | 0.432 |
| LuMayo | 44680 | SQ | 1A | I | 4.50924 | 0 | NA | NA | NA | −0.208 |
| LuMayo | 44693 | SQ | 1B | I | 1.89733 | 1 | NA | NA | NA | −0.491 |
| LuMayo | 48521 | SQ | 1B | I | 5.07871 | 0 | NA | NA | NA | 0.024 |
| LuMayo | 48536 | SQ | 1B | I | 5.07871 | 0 | NA | NA | NA | 0.46 |
| LuMayo | 48549 | SQ | 1A | I | 4.4271 | 0 | NA | NA | NA | −0.268 |
| LuMayo | 48556 | SQ | 1B | I | 5.52225 | 0 | NA | NA | NA | 0.292 |
| LuMayo | 57774 | SQ | 1A | I | 3.38672 | 1 | NA | NA | NA | 0.284 |
| LuMayo | 76981 | SQ | 1B | I | 1.80424 | 1 | NA | NA | NA | 0.253 |
| LuMayo | 86011 | SQ | 1A | I | 1.69747 | 1 | NA | NA | NA | −0.326 |
| LuMayo | 86043 | SQ | 1A | I | 0.87611 | 1 | NA | NA | NA | −0.463 |
| LuWashU | 3196 | AD | 1B | I | 3.37577 | 0 | NA | NA | NA | 0.279 |
| LuWashU | 3197 | AD | 1B | I | 3.55647 | 1 | NA | NA | NA | −0.271 |
| LuWashU | 3200 | AD | 1B | I | 0.91992 | 1 | NA | NA | NA | 0.702 |
| LuWashU | 3202 | AD | 1B | I | 4.96099 | 0 | NA | NA | NA | −0.042 |
| LuWashU | 3205 | AD | 1B | I | 3.19233 | 0 | NA | NA | NA | 0.532 |
| LuWashU | 3210 | AD | 1B | I | 1.80151 | 1 | NA | NA | NA | 0.48 |
| LuWashU | 3211 | AD | 1B | I | 5.04312 | 0 | NA | NA | NA | 0.465 |
| LuWashU | 3213 | AD | 1B | I | 5.45654 | 0 | NA | NA | NA | −0.071 |
| LuWashU | 3218 | AD | 1B | I | 4.95277 | 0 | NA | NA | NA | 1.081 |
| LuWashU | 3223 | AD | 1B | I | 2.70226 | 0 | NA | NA | NA | 0.004 |
| LuWashU | 3226 | AD | 1B | I | 2.20671 | 1 | NA | NA | NA | 0.53 |
| LuWashU | 3227 | AD | 1B | I | 2.20671 | 1 | NA | NA | NA | −0.568 |
| LuWashU | 3229 | AD | 1B | I | 0.14784 | 1 | NA | NA | NA | 0.095 |
| LuWashU | 3230 | AD | 1B | I | 6.23135 | 0 | NA | NA | NA | 0.501 |
| LuWashU | 3198 | SQ | 1B | I | 2.3436 | 0 | NA | NA | NA | 0.544 |
| LuWashU | 3199 | SQ | 1B | I | 6.62286 | 0 | NA | NA | NA | −0.254 |
| LuWashU | 3201 | SQ | 1B | I | 2.26694 | 0 | NA | NA | NA | 0.081 |
| LuWashU | 3203 | SQ | 1B | I | 1.51951 | 0 | NA | NA | NA | −0.192 |
| LuWashU | 3204 | SQ | 1B | I | 2.89117 | 1 | NA | NA | NA | −0.435 |
| LuWashU | 3206 | SQ | 1B | I | 3.38398 | 0 | NA | NA | NA | −0.038 |
| LuWashU | 3208 | SQ | 1B | I | 5.15537 | 0 | NA | NA | NA | −0.229 |
| LuWashU | 3209 | SQ | 1B | I | 0.92539 | 0 | NA | NA | NA | 1.441 |
| LuWashU | 3214 | SQ | 1B | I | 0.84052 | 1 | NA | NA | NA | −0.115 |
| LuWashU | 3215 | SQ | 1B | I | 1.13621 | 0 | NA | NA | NA | 0.037 |
| LuWashU | 3216 | SQ | 1B | I | 4.78576 | 0 | NA | NA | NA | −0.169 |
| LuWashU | 3217 | SQ | 1B | I | 5.81246 | 0 | NA | NA | NA | 0.256 |
| LuWashU | 3220 | SQ | 1B | I | 4.51198 | 0 | NA | NA | NA | −0.121 |
| LuWashU | 3221 | SQ | 1B | I | 6.40657 | 0 | NA | NA | NA | −0.026 |
| LuWashU | 3224 | SQ | 1B | I | 5.84805 | 0 | NA | NA | NA | −0.211 |
| LuWashU | 3225 | SQ | 1B | I | 3.94798 | 0 | NA | NA | NA | −0.233 |
| LuWashU | 3228 | SQ | 1B | I | 4.44627 | 0 | NA | NA | NA | −0.004 |
| LuWashU | 3231 | SQ | 1B | I | 4.67899 | 0 | NA | NA | NA | −0.343 |

| Study | ID | HIF1A | CCT3 | MAFK | HLADPB1 | RNF5 | mSD |
|---|---|---|---|---|---|---|---|
| UHN | B007 | −0.909 | −0.340 | 0.895 | −0.578 | 0.272 | 1 |
| UHN | B013 | 1.524 | 0.130 | −0.081 | 0.390 | −0.769 | 0 |
| UHN | B019 | 0.249 | −0.160 | 0.555 | −1.203 | −0.273 | 1 |
| UHN | B033 | −2.516 | 1.141 | NA | −0.013 | 0.346 | 1 |
| UHN | B048 | −0.931 | 1.061 | NA | −0.135 | −0.117 | 1 |
| UHN | B067 | NA | −1.037 | −0.452 | −0.760 | 0.563 | 1 |
| UHN | B084 | −0.439 | 0.892 | 0.519 | 0.126 | 0.033 | 1 |
| UHN | L005 | 0.104 | 0.081 | 0.156 | 0.186 | −1.176 | 1 |
| UHN | L009 | 0.745 | −0.620 | −0.372 | 1.696 | −0.477 | 1 |
| UHN | L012 | 1.191 | 0.831 | 1.645 | −0.428 | −1.333 | 0 |
| UHN | L018 | −1.248 | −0.444 | 0.163 | 0.538 | −0.243 | 1 |
| UHN | L023 | 0.369 | 0.257 | −0.650 | −0.490 | −0.373 | 1 |
| UHN | L027 | −0.018 | −0.036 | 0.546 | 0.118 | −0.684 | 1 |
| UHN | L028 | 1.119 | 0.807 | −0.707 | −2.090 | −0.243 | 0 |
| UHN | L030 | 1.030 | −0.440 | 0.571 | −0.455 | 0.260 | 0 |
| UHN | L047 | 0.330 | 1.009 | −0.116 | −4.254 | 0.984 | 0 |
| UHN | L049 | 0.476 | −1.522 | 0.263 | −1.186 | −1.036 | 0 |
| UHN | L051 | −0.233 | −0.277 | −0.696 | −1.390 | −0.419 | 1 |
| UHN | L052 | 0.605 | 0.351 | −0.665 | −0.965 | 1.228 | 0 |
| UHN | L056 | 0.750 | −0.746 | NA | 0.565 | −0.205 | 1 |
| UHN | L058 | 0.000 | 0.282 | 0.270 | 0.061 | −1.850 | 0 |
| UHN | L059 | NA | 0.271 | 1.355 | 0.893 | −0.502 | 1 |
| UHN | L061 | −0.141 | 1.507 | 1.119 | 0.157 | 0.063 | 0 |
| UHN | L062 | 0.027 | −0.754 | 0.731 | −1.056 | −0.618 | 1 |
| UHN | L066 | −8.024 | 0.147 | 1.149 | 0.582 | 0.065 | 1 |
| UHN | L078 | 0.958 | −0.287 | −1.143 | −3.552 | −0.601 | 0 |
| UHN | L083 | −0.622 | 0.172 | −2.221 | −0.032 | −0.078 | 1 |
| UHN | L086 | −0.083 | 0.132 | 0.007 | 0.163 | −0.833 | 1 |
| UHN | L093 | −0.493 | −0.676 | 1.244 | −1.833 | −0.202 | 1 |
| UHN | L095 | NA | −0.012 | 0.384 | −1.914 | −0.158 | 0 |
| UHN | L098 | 1.589 | 0.686 | 0.835 | −2.131 | −0.674 | 0 |
| UHN | L105 | 0.866 | −0.733 | −0.057 | 0.944 | 0.847 | 0 |
| UHN | L106 | −1.251 | 0.194 | −5.661 | 0.525 | −0.391 | 1 |
| UHN | L112 | −1.256 | 0.477 | −0.864 | −2.690 | 0.046 | 1 |
| UHN | L115 | 0.642 | 0.285 | −0.804 | −0.077 | −0.189 | 1 |
| UHN | L116 | 0.253 | −0.347 | 0.354 | 0.309 | 0.622 | 1 |
| UHN | L120 | −0.099 | −0.542 | NA | 0.164 | 2.362 | 1 |
| UHN | L123 | 0.338 | −0.604 | −0.035 | −0.471 | 0.543 | 1 |
| UHN | L127 | 1.181 | −0.171 | 0.316 | −1.289 | −4.817 | 0 |
| UHN | L133 | 2.165 | −0.607 | NA | −0.934 | 5.498 | 0 |
| UHN | L148 | −0.341 | −0.166 | 1.296 | −1.097 | 0.341 | 1 |
| UHN | L164 | 0.281 | 0.352 | −0.323 | 2.178 | 1.637 | 1 |
| UHN | L174 | −0.361 | 1.294 | NA | −2.207 | 0.390 | 0 |
| UHN | L175 | −1.783 | 0.259 | −0.625 | 0.672 | 0.768 | 1 |
| UHN | L182 | −0.723 | −1.297 | −1.921 | −1.379 | −1.055 | 1 |
| UHN | L191 | 0.660 | −1.624 | −0.169 | −1.574 | −1.041 | 0 |
| UHN | L195 | 0.537 | −0.204 | −1.200 | −1.851 | −0.235 | 1 |
| UHN | L197 | −0.056 | 0.181 | −1.103 | −0.097 | −0.639 | 1 |
| UHN | L201 | 1.431 | 1.462 | NA | −1.188 | −2.179 | 0 |
| UHN | L212 | −0.163 | −0.010 | −2.586 | 0.415 | −0.165 | 1 |
| UHN | L214 | −0.128 | −0.490 | 0.205 | −1.942 | −0.292 | 1 |
| UHN | L218 | 1.362 | 0.241 | −1.079 | −1.584 | −0.785 | 0 |
| UHN | L222 | −2.963 | 0.233 | NA | 0.090 | −0.061 | 1 |
| UHN | P001 | −1.282 | −1.075 | −0.205 | −0.053 | −0.118 | 1 |
| UHN | P002 | −1.171 | −1.093 | −0.552 | −0.287 | −0.260 | 1 |
| UHN | P004 | −9.886 | 0.785 | 0.229 | −0.184 | −0.102 | 1 |
| UHN | P006 | −0.279 | 0.000 | −0.462 | −0.152 | 0.000 | 0 |
| UHN | P009 | 1.096 | 0.611 | 0.784 | 0.525 | −0.886 | 0 |
| UHN | P010 | 5.562 | −1.343 | 0.717 | 0.070 | 0.467 | 0 |
| UHN | P017 | −0.503 | 0.608 | −5.755 | 0.401 | 0.006 | 1 |
| UHN | P020 | 0.698 | 2.274 | NA | −0.341 | −0.015 | 0 |
| UHN | P026 | −0.421 | 0.015 | 1.138 | 0.421 | 0.603 | 1 |
| UHN | P030 | −1.949 | −1.120 | 0.395 | 1.191 | −0.041 | 1 |
| UHN | P031 | 1.920 | 2.160 | 0.621 | 0.095 | −0.015 | 0 |
| UHN | P042 | 0.135 | −0.097 | 0.527 | 0.557 | 0.684 | 1 |
| UHN | P043 | 1.036 | −0.305 | 0.299 | 0.426 | 0.433 | 0 |
| UHN | P046 | 1.304 | 0.458 | 1.047 | 1.231 | 0.241 | 0 |
| UHN | P080 | −0.467 | 0.118 | −0.485 | −0.334 | 0.918 | 1 |
| UHN | P081 | −0.291 | −0.363 | 1.053 | 0.933 | 0.436 | 0 |
| UHN | P085 | −1.347 | −0.079 | NA | 1.515 | −0.744 | 1 |
| UHN | P086 | NA | −0.988 | 1.166 | 1.012 | −1.308 | 0 |
| UHN | P089 | 2.044 | 2.092 | 1.663 | −1.347 | −0.263 | 0 |
| UHN | P091 | 1.018 | −0.129 | NA | 0.844 | 0.096 | 0 |
| UHN | P092 | 0.254 | −0.336 | 0.716 | 0.482 | 0.502 | 1 |
| UHN | P093 | 1.085 | −0.023 | −0.879 | −2.366 | −0.192 | 0 |
| UHN | P100 | 0.014 | −0.147 | 0.559 | 0.206 | 0.771 | 1 |
| UHN | P106 | 0.950 | 0.486 | −0.244 | −1.378 | 0.477 | 0 |
| UHN | P108 | 3.410 | 1.595 | 2.524 | 1.482 | 0.172 | 0 |
| UHN | P114 | −1.341 | −0.484 | −1.059 | 0.095 | 0.012 | 1 |
| UHN | P118 | NA | −0.312 | 0.332 | 1.862 | 0.793 | 1 |
| UHN | P119 | −0.866 | 0.556 | 1.778 | 2.299 | 0.757 | 1 |
| UHN | P123 | −0.368 | 1.059 | 0.058 | 0.725 | 1.121 | 1 |
| UHN | P124 | −1.405 | −0.784 | 0.622 | 0.430 | 0.626 | 1 |
| UHN | P130 | −0.452 | −0.138 | NA | 0.901 | 0.347 | 1 |
| UHN | P131 | 0.741 | −0.549 | 0.014 | −0.143 | −0.146 | 1 |
| UHN | P132 | −0.005 | −0.006 | 1.300 | −0.136 | −0.788 | 0 |
| UHN | P133 | 1.443 | 0.436 | 1.685 | 0.950 | 1.935 | 0 |
| UHN | P135 | 0.415 | 0.145 | 0.142 | −0.141 | −0.125 | 0 |
| UHN | P136 | 0.254 | −0.247 | −0.162 | 1.151 | 1.101 | 1 |
| UHN | P140 | −0.317 | −0.751 | −1.092 | 0.660 | −0.370 | 1 |
| UHN | P143 | 0.815 | 1.551 | NA | 0.565 | 0.809 | 0 |
| UHN | P147 | 0.085 | 0.796 | NA | 1.777 | 0.154 | 0 |
| UHN | P149 | −0.634 | 0.359 | −0.330 | 1.533 | 0.778 | 1 |
| UHN | P152 | −0.844 | 1.359 | −0.797 | −0.271 | 1.082 | 0 |
| UHN | P158 | 0.629 | 2.918 | NA | −2.021 | 0.581 | 0 |
| UHN | P159 | 1.874 | 0.801 | −0.689 | −0.937 | −0.315 | 0 |
| UHN | P163 | −0.838 | −0.940 | 0.138 | 1.743 | 0.243 | 1 |
| UHN | P164 | −0.459 | 0.213 | −0.681 | 0.823 | 0.174 | 1 |
| UHN | P166 | 2.020 | 0.427 | 0.102 | −1.087 | −1.289 | 0 |
| UHN | P167 | NA | 1.345 | NA | 1.873 | 1.185 | 0 |
| UHN | P168 | 1.300 | 1.424 | 2.181 | −2.148 | 0.772 | 0 |
| UHN | P169 | −1.234 | 1.763 | −0.347 | −1.540 | 1.385 | 0 |
| UHN | P171 | 0.450 | 2.661 | 1.299 | −0.951 | 0.965 | 0 |
| UHN | P173 | −0.143 | 1.654 | 0.703 | −0.545 | 0.736 | 0 |
| UHN | P174 | −0.826 | −0.357 | −0.890 | 0.053 | 0.079 | 1 |
| UHN | P177 | 0.429 | −0.345 | −1.740 | −0.841 | 0.950 | 1 |
| UHN | P181 | 1.065 | −0.400 | −0.062 | −0.772 | −0.863 | 1 |
| UHN | P185 | −0.655 | 0.007 | −0.810 | −0.257 | 0.074 | 1 |
| UHN | P186 | 0.524 | −0.034 | 2.139 | −1.400 | −0.772 | 0 |
| UHN | P188 | −0.509 | −0.287 | −0.204 | 1.710 | 0.781 | 1 |
| UHN | P189 | −0.011 | 0.231 | −0.027 | −0.905 | −0.699 | 1 |
| UHN | P191 | −0.378 | −0.575 | −0.991 | −0.166 | −1.059 | 1 |
| UHN | P196 | −0.749 | −0.099 | NA | 0.567 | −0.373 | 1 |
| UHN | P201 | −0.469 | −0.664 | 0.799 | 0.205 | −0.270 | 1 |
| UHN | P204 | 0.464 | 0.388 | NA | 1.166 | −0.520 | 1 |
| UHN | P205 | 0.870 | 0.482 | 0.667 | 0.091 | 0.374 | 0 |
| UHN | P209 | 1.195 | 1.722 | NA | −0.131 | 0.290 | 0 |
| UHN | P210 | 2.622 | 1.125 | −0.025 | 1.039 | 0.015 | 0 |
| UHN | P214 | 0.383 | 0.962 | NA | 0.689 | 0.410 | 1 |
| UHN | P215 | 2.139 | −0.298 | NA | 0.756 | 0.170 | 0 |
| UHN | P218 | 0.901 | 1.750 | 0.122 | −1.328 | 0.296 | 0 |
| UHN | P221 | 0.923 | 0.003 | −0.216 | 0.482 | 0.018 | 0 |
| UHN | P223 | −1.758 | −0.303 | 1.031 | −0.013 | 0.936 | 1 |
| UHN | P224 | −2.922 | −0.255 | −0.007 | 0.064 | 1.078 | 1 |
| UHN | P226 | −0.109 | −0.950 | −0.719 | 0.573 | −0.380 | 1 |
| UHN | P227 | −1.306 | 0.591 | −0.906 | −2.344 | 0.683 | 1 |
| UHN | P228 | 1.427 | −0.143 | −0.294 | −0.502 | −0.443 | 1 |
| UHN | P230 | −0.968 | 0.932 | NA | −0.310 | 1.403 | 1 |
| UHN | P238 | −0.703 | 0.281 | −1.328 | 0.904 | 0.167 | 1 |
| UHN | P239 | 0.747 | −0.575 | −2.191 | −0.542 | −1.279 | 1 |
| UHN | P240 | 0.285 | 0.366 | −0.137 | 1.497 | 0.287 | 1 |
| UHN | P241 | −1.483 | −0.882 | −0.292 | 0.000 | 0.064 | 1 |
| UHN | P243 | −1.047 | −0.274 | 1.446 | 1.914 | −0.285 | 1 |
| UHN | P245 | −0.478 | −0.407 | 1.210 | 1.472 | 1.029 | 1 |
| UHN | P248 | −0.857 | −0.449 | −0.153 | −0.370 | 0.214 | 1 |
| UHN | P250 | −3.205 | −0.547 | 0.844 | 1.808 | −0.234 | 1 |
| UHN | P253 | −2.739 | 0.079 | NA | −0.672 | 0.134 | 1 |
| UHN | P254 | −0.211 | −1.192 | −0.812 | 0.218 | −0.640 | 1 |
| UHN | P257 | −0.426 | −0.962 | −0.142 | −0.433 | −0.886 | 1 |
| UHN | P274 | −1.506 | −1.105 | −0.424 | 1.323 | −0.418 | 1 |
| UHN | P275 | −0.351 | −0.005 | −0.945 | 0.905 | −0.543 | 1 |
| UHN | P278 | 1.186 | −1.258 | −0.604 | 0.044 | −1.287 | 1 |
| UHN | P284 | 0.338 | 0.036 | 0.225 | 0.567 | −0.186 | 1 |
| UHN | P287 | 1.107 | 0.664 | NA | −0.360 | 1.099 | 1 |
| UHN | P295 | 0.703 | 1.588 | 2.053 | −0.980 | −0.134 | 0 |
| UHN | P302 | −0.656 | 1.781 | NA | −0.980 | −0.045 | 1 |
| UHN | P313 | −0.778 | −0.305 | 0.421 | −1.116 | 0.126 | 1 |
| MI02 | AD10 | −0.462 | −0.284 | NA | 0.601 | 0.000 | NA |
| MI02 | AD2 | 0.088 | 0.144 | NA | −0.662 | 0.001 | NA |
| MI02 | AD3 | 0.446 | 0.307 | NA | −0.332 | −0.025 | NA |
| MI02 | AD5 | −0.035 | −0.096 | NA | 0.947 | 0.053 | NA |
| MI02 | AD6 | −0.477 | −0.524 | NA | −0.293 | 0.165 | NA |
| MI02 | AD7 | 0.198 | 0.498 | NA | 0.468 | −0.140 | NA |
| MI02 | AD8 | −0.301 | −0.675 | NA | −0.268 | 0.239 | NA |
| MI02 | L01 | 0.178 | −0.299 | NA | −1.490 | −0.026 | NA |
| MI02 | L02 | 0.996 | −0.375 | NA | 1.013 | 0.176 | NA |
| MI02 | L04 | 0.277 | 0.261 | NA | −0.603 | −0.096 | NA |
| MI02 | L05 | −0.316 | 0.093 | NA | 0.048 | 0.375 | NA |
| MI02 | L06 | 0.579 | 0.712 | NA | −0.537 | 0.104 | NA |
| MI02 | L08 | −0.096 | 0.170 | NA | 0.390 | −0.084 | NA |
| MI02 | L09 | 0.794 | 0.135 | NA | 0.521 | −0.258 | NA |
| MI02 | L100 | 0.190 | −1.103 | NA | 0.810 | 0.291 | NA |
| MI02 | L101 | −0.431 | −0.812 | NA | 0.565 | 0.192 | NA |
| MI02 | L102 | 0.449 | −0.384 | NA | −0.310 | 1.019 | NA |
| MI02 | L103 | −0.409 | −0.566 | NA | −0.256 | 0.146 | NA |
| MI02 | L104 | −0.254 | −0.396 | NA | 0.216 | 0.269 | NA |
| MI02 | L105 | −0.362 | 0.678 | NA | 0.773 | 0.280 | NA |
| MI02 | L106 | −0.073 | 0.052 | NA | 0.950 | −0.215 | NA |
| MI02 | L107 | −0.115 | −0.864 | NA | −0.007 | −0.111 | NA |
| MI02 | L108 | 0.140 | 0.173 | NA | −1.244 | 0.444 | NA |
| MI02 | L11 | −0.536 | −0.475 | NA | −0.544 | 0.166 | NA |
| MI02 | L111 | −0.191 | 0.060 | NA | −0.134 | 0.170 | NA |
| MI02 | L12 | −0.493 | −0.222 | NA | −0.366 | 0.231 | NA |
| MI02 | L13 | −0.104 | −0.463 | NA | 0.308 | 0.000 | NA |
| MI02 | L17 | 0.386 | 0.209 | NA | −1.176 | −0.120 | NA |
| MI02 | L18 | −0.683 | 0.280 | NA | 0.049 | 0.053 | NA |
| MI02 | L19 | −0.233 | 0.001 | NA | 0.426 | −0.341 | NA |
| MI02 | L20 | −0.181 | −1.006 | NA | −0.359 | 0.283 | NA |
| MI02 | L22 | −0.087 | −1.085 | NA | −0.429 | 0.485 | NA |
| MI02 | L23 | 0.322 | 0.849 | NA | 0.468 | −0.278 | NA |
| MI02 | L24 | 0.319 | 0.283 | NA | 0.303 | −0.082 | NA |
| MI02 | L25 | −0.042 | 0.295 | NA | 0.215 | 0.466 | NA |
| MI02 | L26 | 0.387 | 1.136 | NA | −0.740 | 0.020 | NA |
| MI02 | L27 | −0.267 | 1.667 | NA | −1.621 | 0.600 | NA |
| MI02 | L30 | −0.461 | −0.788 | NA | 0.323 | 0.332 | NA |
| MI02 | L31 | 0.472 | −0.314 | NA | 0.284 | 0.032 | NA |
| MI02 | L33 | 0.048 | 1.428 | NA | −1.156 | 0.386 | NA |
| MI02 | L34 | −0.123 | 0.495 | NA | 0.666 | −0.102 | NA |
| MI02 | L35 | 1.124 | 0.268 | NA | −0.156 | −0.479 | NA |
| MI02 | L36 | 0.337 | 0.929 | NA | −0.458 | −0.321 | NA |
| MI02 | L37 | 0.127 | 1.172 | NA | −0.825 | −0.206 | NA |
| MI02 | L38 | 0.322 | −0.239 | NA | 0.403 | −0.371 | NA |
| MI02 | L40 | 0.002 | 1.185 | NA | −1.570 | −0.198 | NA |
| MI02 | L41 | −0.096 | 0.835 | NA | −0.484 | −0.175 | NA |
| MI02 | L42 | −0.255 | −0.536 | NA | −0.069 | 0.264 | NA |
| MI02 | L43 | −0.196 | 0.528 | NA | −0.555 | −0.007 | NA |
| MI02 | L45 | 0.014 | 0.839 | NA | 0.350 | −0.285 | NA |
| MI02 | L46 | −0.133 | −0.008 | NA | −0.239 | −0.073 | NA |
| MI02 | L47 | 0.180 | 0.733 | NA | −0.313 | −0.181 | NA |
| MI02 | L48 | 0.044 | 0.013 | NA | −0.525 | 0.250 | NA |
| MI02 | L49 | 0.178 | −0.300 | NA | 0.019 | 0.058 | NA |
| MI02 | L50 | −0.101 | −0.225 | NA | −0.266 | −0.129 | NA |
| MI02 | L52 | −0.386 | −0.459 | NA | −0.810 | 0.290 | NA |
| MI02 | L53 | −0.083 | −1.016 | NA | 0.007 | 0.067 | NA |
| MI02 | L54 | 0.825 | −0.007 | NA | −0.789 | −0.453 | NA |
| MI02 | L56 | −0.049 | 0.731 | NA | −0.152 | −0.303 | NA |
| MI02 | L57 | 1.366 | 0.788 | NA | 0.202 | −0.086 | NA |
| MI02 | L59 | 0.218 | 1.698 | NA | −0.682 | 0.065 | NA |
| MI02 | L61 | 0.078 | −0.031 | NA | −1.232 | 0.468 | NA |
| MI02 | L62 | −0.002 | 0.138 | NA | −0.132 | 0.223 | NA |
| MI02 | L64 | 0.339 | −0.106 | NA | −0.566 | 0.308 | NA |
| MI02 | L65 | −0.024 | 0.809 | NA | 0.450 | −0.103 | NA |
| MI02 | L76 | −0.253 | 0.721 | NA | −2.462 | 0.839 | NA |
| MI02 | L78 | −0.097 | −0.266 | NA | 0.017 | −0.021 | NA |
| MI02 | L79 | 0.094 | 1.250 | NA | −0.417 | 0.269 | NA |
| MI02 | L80 | 0.116 | 1.187 | NA | −1.652 | 0.292 | NA |
| MI02 | L81 | 1.093 | −0.107 | NA | 0.174 | 1.678 | NA |
| MI02 | L82 | −0.015 | −0.340 | NA | 0.271 | −0.234 | NA |
| MI02 | L83 | 0.297 | 0.109 | NA | −0.916 | −0.014 | NA |
| MI02 | L84 | −0.224 | −0.221 | NA | 0.923 | 0.031 | NA |
| MI02 | L85 | −0.008 | 0.896 | NA | −1.333 | 0.159 | NA |
| MI02 | L86 | −0.273 | −0.285 | NA | 0.527 | −0.011 | NA |
| MI02 | L87 | 0.136 | 0.367 | NA | 0.274 | 0.061 | NA |
| MI02 | L88 | 1.111 | 0.349 | NA | 0.932 | −1.018 | NA |
| MI02 | L89 | 0.732 | −0.153 | NA | 0.291 | −1.649 | NA |
| MI02 | L90 | 0.913 | 0.247 | NA | 0.608 | −0.090 | NA |
| MI02 | L91 | 0.236 | 0.370 | NA | −0.930 | −0.215 | NA |
| MI02 | L92 | 0.038 | 0.382 | NA | −1.412 | 0.423 | NA |
| MI02 | L94 | 0.070 | 0.988 | NA | −0.513 | −0.127 | NA |
| MI02 | L95 | −0.029 | 0.420 | NA | −0.271 | −0.180 | NA |
| MI02 | L96 | −0.004 | −0.583 | NA | 0.233 | 0.204 | NA |
| MI02 | L97 | −0.394 | −0.001 | NA | 0.319 | −0.055 | NA |
| MI02 | L99 | 0.062 | −0.449 | NA | −0.851 | 0.771 | NA |
| MIT | AD111 | −0.39 | 0.115 | 0.029 | 0.193942 | −0.23 | NA |
| MIT | AD114 | 0.271 | 0.314 | −0.07 | 0.563618 | −0.13 | NA |
| MIT | AD119 | −0.34 | −0.56 | −0.01 | 0.85794 | −0.35 | NA |
| MIT | AD123 | 0.111 | −0.16 | −0.17 | 0.682795 | −0.18 | NA |
| MIT | AD131 | −0.12 | 0.574 | −0.22 | −1.44481 | 0.025 | NA |
| MIT | AD136 | 0.221 | −0.21 | −0.05 | 0.422367 | 0.075 | NA |
| MIT | AD162 | 0.223 | 0 | −0.15 | 0.242173 | −0.27 | NA |
| MIT | AD167 | −0.36 | 0.422 | 0.202 | −0.00429 | 0.021 | NA |
| MIT | AD170 | −0.2 | 0.579 | −0.06 | −0.72557 | −0.04 | NA |
| MIT | AD172 | −0.03 | 0.13 | 0.377 | 0.204315 | 0.337 | NA |
| MIT | AD183 | −0.21 | 0.605 | −0.03 | −0.08333 | −0.07 | NA |
| MIT | AD186 | −0.31 | 1.493 | 0.729 | −1.29805 | 0.137 | NA |
| MIT | AD202 | −0.42 | −0.81 | 0.319 | −0.11378 | 0.152 | NA |
| MIT | AD203 | −0.38 | −0.04 | 0.445 | 0.390427 | 0.25 | NA |
| MIT | AD210 | −0.1 | −0.05 | 0.46 | 0.131801 | −0.03 | NA |
| MIT | AD212 | 0.669 | −0.29 | −0.12 | 0.663692 | −0.26 | NA |
| MIT | AD218 | −0.56 | −0.72 | 0.329 | −0.9192 | 0.18 | NA |
| MIT | AD221 | −0.64 | −0.55 | 0.273 | −0.45563 | 0.01 | NA |
| MIT | AD224 | −0.01 | 0.205 | 0.341 | 0.204124 | 0.309 | NA |
| MIT | AD226 | −0.45 | −0.81 | 0.297 | 0.712732 | 0.542 | NA |
| MIT | AD230 | −0.55 | 0.121 | −0.28 | −0.28401 | −0.28 | NA |
| MIT | AD232 | −0.55 | −0.67 | 0.189 | 0.450015 | 0.335 | NA |
| MIT | AD234 | 0.152 | −0.56 | 0.125 | −1.08505 | 0.084 | NA |
| MIT | AD239 | −0.14 | −0.11 | 0.578 | −0.65691 | 0.039 | NA |
| MIT | AD240 | −0.41 | −0.56 | 0.143 | 0.87961 | 0.154 | NA |
| MIT | AD243 | −0.19 | −1.06 | 0.101 | 1.409709 | 0.052 | NA |
| MIT | AD247 | 0.287 | −0.45 | −0.34 | 0.842517 | −0.07 | NA |
| MIT | AD250 | 0.314 | −0.28 | 0.012 | 0.099629 | −0.1 | NA |
| MIT | AD253 | 0.218 | 0.195 | 0.044 | 0.663907 | −0.07 | NA |
| MIT | AD255 | 0.278 | 0.033 | −0.34 | 0.450156 | −0.31 | NA |
| MIT | AD261 | 0.928 | −0.4 | −0.23 | 0.134347 | −0.18 | NA |
| MIT | AD267 | −0.77 | −0.6 | −0.4 | 1.706393 | −0.25 | NA |
| MIT | AD268 | 0.242 | 0.929 | 0.074 | −0.52087 | 0.039 | NA |
| MIT | AD294 | 0.091 | −0.85 | −0.14 | 1.241865 | 9E−04 | NA |
| MIT | AD295 | 0.554 | 0.002 | −0.26 | −0.27159 | −0.5 | NA |
| MIT | AD305 | 0.55 | −0.01 | −0.55 | 0.590131 | 0.107 | NA |
| MIT | AD308 | 0.671 | 0.217 | 0.037 | 0.632728 | −0.04 | NA |
| MIT | AD311 | 0.854 | −0.26 | 0.151 | 0.328915 | 0.12 | NA |
| MIT | AD315 | 0.961 | 0.325 | 0.062 | 0.022571 | 0.006 | NA |
| MIT | AD317 | −0.13 | −0.39 | 0.138 | 2.051241 | −0.01 | NA |
| MIT | AD318 | −0.24 | −0.22 | 0.218 | 0.177935 | 0.303 | NA |
| MIT | AD320 | −0.4 | 0.165 | 0.153 | −1.62951 | 0.213 | NA |
| MIT | AD327 | −0.12 | 0.174 | 0.366 | −0.19861 | 0.102 | NA |
| MIT | AD331 | 0.356 | 0.527 | 0.56 | −1.52274 | −0.11 | NA |
| MIT | AD335 | 0.297 | 0.096 | −0.27 | −1.50253 | −0.24 | NA |
| MIT | AD337 | 0.688 | −0.02 | −0.2 | 0.579281 | −0.14 | NA |
| MIT | AD338 | −0.04 | −0.79 | 0.347 | 0.758845 | 0.482 | NA |
| MIT | AD346 | 0.189 | −0.88 | 0.009 | 0.570113 | −0.16 | NA |
| MIT | AD347 | −0.52 | −0.43 | 0.128 | 0.9021 | 0.063 | NA |
| MIT | AD353 | −0.46 | 0.242 | 0.035 | 1.20298 | −0.12 | NA |
| MIT | AD356 | 0.086 | −0.29 | −0.44 | 1.713857 | −0.07 | NA |
| MIT | AD367 | 0.25 | 0.476 | −0.07 | −0.98474 | −0.02 | NA |
| MIT | AD368 | −0.21 | 0.583 | 0.737 | −0.25694 | 0.025 | NA |
| MIT | AD379 | −0.39 | −0.21 | 0.478 | −0.62942 | −0.29 | NA |
| MIT | AD043 | −0.79 | −0.22 | −0.28 | −0.65403 | −0.02 | NA |
| MIT | AD115 | 0.176 | 0.229 | 0.083 | −0.0796 | −0.04 | NA |
| MIT | AD118 | 0.739 | 0.027 | −0.42 | 0.004901 | −0.37 | NA |
| MIT | AD120 | 0.515 | −0.48 | 0.484 | −0.87317 | −0.16 | NA |
| MIT | AD122 | −0.52 | −0.48 | 0.025 | 0.470954 | −0.15 | NA |
| MIT | AD127 | 0.319 | −0.35 | −0.24 | 0.631518 | 0.074 | NA |
| MIT | AD130 | −0.46 | 0.192 | 0.068 | −0.81572 | 0.257 | NA |
| MIT | AD157 | −0.34 | −0.07 | −0.2 | 0.357903 | −0.3 | NA |
| MIT | AD158 | 0.786 | 0.177 | 0.194 | −1.01954 | 0.177 | NA |
| MIT | AD159 | 0.827 | 0.812 | 0.205 | −0.24666 | 0.087 | NA |
| MIT | AD163 | −0.54 | 0.655 | 0.426 | −0.63086 | −0.02 | NA |
| MIT | AD164 | 1.194 | −0.09 | −0.31 | 0.669098 | −0.2 | NA |
| MIT | AD169 | −0.2 | −0.34 | 0.276 | 0.110231 | 0.125 | NA |
| MIT | AD173 | −0.1 | 0.511 | 0.344 | −0.39972 | 0.282 | NA |
| MIT | AD177 | −0.15 | 0.069 | −0.08 | 0.392346 | −0.18 | NA |
| MIT | AD178 | −0.53 | 0.378 | 0.417 | −1.26796 | −0.01 | NA |
| MIT | AD179 | 0.256 | 0.328 | 0.371 | −0.29943 | 0.094 | NA |
| MIT | AD185 | 0.253 | 0.538 | 0.108 | −1.82272 | 0.039 | NA |
| MIT | AD187 | 0.37 | 0.209 | −0.07 | 0.495898 | 0.069 | NA |
| MIT | AD188 | −0.46 | 0.59 | 0.182 | 0.120879 | 0.424 | NA |
| MIT | AD201 | 0.507 | 0.791 | 0.374 | −0.74763 | −0.16 | NA |
| MIT | AD207 | −0.28 | −0.39 | 0.297 | 0.650388 | 0.101 | NA |
| MIT | AD208 | −0.16 | −0.06 | 0.453 | −0.22581 | 0.359 | NA |
| MIT | AD213 | −0.48 | −0.3 | −0.17 | 0.97115 | 0.08 | NA |
| MIT | AD225 | 0.141 | −0.39 | −0.25 | 0.674158 | −0.24 | NA |
| MIT | AD228 | −0.37 | 0.135 | 0.317 | −0.55952 | 0.028 | NA |
| MIT | AD236 | 0.709 | 0.435 | −0.18 | −0.47393 | −0.08 | NA |
| MIT | AD238 | 0.009 | −0.06 | 0.006 | 1.017882 | 0.272 | NA |
| MIT | AD241 | −0.31 | 0.276 | −0.16 | 0.504429 | 0.009 | NA |
| MIT | AD249 | 0.495 | 0.594 | −0.08 | −0.3981 | 0.133 | NA |
| MIT | AD252 | 0.474 | 0.441 | −0.05 | 0 | 0.096 | NA |
| MIT | AD258 | 0.383 | −0.05 | 0.039 | 0.010844 | −0.1 | NA |
| MIT | AD259 | 0.592 | −0.78 | −0.23 | 0.589045 | −0.1 | NA |
| MIT | AD260 | 0.499 | −0.09 | −0.44 | 0.826039 | −0.13 | NA |
| MIT | AD262 | −0.07 | −0.82 | 0 | 1.00825 | −0.13 | NA |
| MIT | AD266 | −0.17 | −0.75 | −0.25 | 0.660582 | 0.01 | NA |
| MIT | AD269 | 0.02 | −0.59 | −0.08 | 1.307848 | −0.22 | NA |
| MIT | AD275 | 1.036 | 0.099 | −0.34 | −0.92995 | −0.48 | NA |
| MIT | AD276 | 0.279 | 0.707 | 0.135 | 0.196825 | 0.025 | NA |
| MIT | AD277 | 0.053 | 1.024 | 0.479 | −0.30603 | 0.134 | NA |
| MIT | AD283 | −0.09 | −0.6 | −0.24 | −0.13893 | −0.39 | NA |
| MIT | AD285 | −0.6 | −0.45 | −0.02 | 0.523891 | 0.008 | NA |
| MIT | AD287 | −0.13 | −0.17 | −0.87 | −0.17785 | −0.63 | NA |
| MIT | AD296 | 0.021 | 0.49 | 0.05 | 0.201074 | −0.13 | NA |
| MIT | AD299 | 0.541 | 0.549 | −0.23 | 0.230953 | −0 | NA |
| MIT | AD301 | −0.13 | 0.539 | −0.01 | −0.47023 | 0.023 | NA |
| MIT | AD302 | 0.27 | −0.41 | −0.04 | −0.01817 | −0.13 | NA |
| MIT | AD304 | 0.011 | 0.031 | −0.12 | −0.19546 | 0.02 | NA |
| MIT | AD309 | 0.383 | −0.28 | 1.088 | 1.584946 | 0.639 | NA |
| MIT | AD313 | −0.19 | 0.201 | 0.328 | 0.41138 | 0.076 | NA |
| MIT | AD314 | −0.25 | −0.17 | −0.16 | 0.150089 | 0.225 | NA |
| MIT | AD323 | 0.627 | −0.07 | −0.09 | 0.749414 | −0.16 | NA |
| MIT | AD330 | −0.19 | 0.383 | 0.129 | 0.576575 | −0.11 | NA |
| MIT | AD332 | 0.259 | 0.285 | −0.05 | −1.06261 | 0.069 | NA |
| MIT | AD334 | 0.857 | −0.12 | 0.152 | −0.17162 | 0.12 | NA |
| MIT | AD336 | 0.145 | 0.232 | 0.079 | 0.059264 | −0.07 | NA |
| MIT | AD340 | −0.59 | −0.53 | 0.169 | −0.40728 | −0.09 | NA |
| MIT | AD341 | −0.18 | 0.006 | 0.083 | −1.52525 | −0.23 | NA |
| MIT | AD350 | −0.14 | −1.12 | 0.046 | 0.154608 | −0.16 | NA |
| MIT | AD351 | −0.32 | 0.648 | 0.606 | −1.98549 | 0.417 | NA |
| MIT | AD352 | −0.58 | −0.27 | −0.45 | −0.14107 | −0.26 | NA |
| MIT | AD361 | 0.252 | 0.228 | −0.24 | −0.12945 | −0.1 | NA |
| MIT | AD362 | −0.32 | −0.28 | 0.169 | −0.80414 | 0.116 | NA |
| MIT | AD363 | −0.18 | −0.71 | −0.37 | 0.668135 | −0.29 | NA |
| MIT | AD366 | 0.107 | 0.29 | 0.56 | −1.22572 | −0.05 | NA |
| MIT | AD370 | 0.87 | −0.14 | −0.33 | −0.19477 | −0.3 | NA |
| MIT | AD374 | 0.908 | −0.15 | −0.2 | −0.11601 | −0.17 | NA |
| MIT | AD375 | −0.17 | −1.11 | −0.16 | −1.46582 | −0.18 | NA |
| MIT | AD382 | −0.24 | 0.662 | 0.153 | −0.32596 | 0.122 | NA |
| MIT | AD383 | 0.997 | −0.5 | −0.18 | −0.11731 | −0.18 | NA |
| MIT | AD384 | −0.49 | −0.3 | 0.033 | −1.05374 | 0.138 | NA |
| Duke | 97-949 | −0.6 | −1.29 | −0.44 | 1.837807 | −0.74 | NA |
| Duke | 98-292 | −0.82 | −0.35 | −0.9 | 0.291761 | −0.2 | NA |
| Duke | 98-679 | −1.34 | −1.08 | −0.91 | 0.903295 | −0.58 | NA |
| Duke | 99-77 | 0.312 | 0.3 | 0.456 | −1.38028 | −0.78 | NA |
| Duke | 99-55 | 0.523 | 0.641 | 1.677 | −2.86746 | −0.38 | NA |
| Duke | 98-985 | −0.74 | −1.43 | 0.785 | 1.149627 | 0.03 | NA |
| Duke | 98-821 | 0.474 | −0.79 | −0.01 | 0.993017 | −0.17 | NA |
| Duke | 98-853 | 0.65 | 0.378 | 0.471 | −2.15327 | 0.197 | NA |
| Duke | 99-927 | 0.67 | 0.012 | 0.064 | −1.50339 | −0.28 | NA |
| Duke | 00-10 | −0.02 | −0.17 | 0.442 | −0.44538 | 0.09 | NA |
| Duke | 98-506 | 0.628 | 0.479 | 0.201 | −0.74527 | −0.57 | NA |
| Duke | 99-1033 | −1.26 | −1.5 | −0.13 | 2.260116 | −0.23 | NA |
| Duke | 98-320 | 0.647 | 0.559 | −0.91 | −2.32832 | 0.419 | NA |
| Duke | 98-711 | 0.021 | 0.752 | 0.606 | −0.57036 | −0.17 | NA |
| Duke | 98-401 | 0.386 | −0.53 | −0.13 | 0.787941 | −0.99 | NA |
| Duke | 96-3 | −1.31 | −0.59 | 0.779 | −0.30914 | −0.07 | NA |
| Duke | 97-1026 | −0.18 | −0.96 | −0.89 | 1.47251 | 0.117 | NA |
| Duke | 98-933 | −0.11 | 0.679 | 0.831 | −0.61133 | −0.26 | NA |
| Duke | 96-475 | 0.1 | 0.806 | −0.18 | 1.026085 | −0.74 | NA |
| Duke | 99-671 | −0.52 | −0.24 | 0.059 | −0.05234 | 0.132 | NA |
| Duke | 98-683 | −0.51 | −0.48 | 0.861 | −0.73058 | −0.84 | NA |
| Duke | 97-403 | 0.22 | −0.26 | 1.355 | 0.116961 | −0.28 | NA |
| Duke | 97-587 | −0.6 | 0.694 | 0.394 | 0.923019 | 0.032 | NA |
| Duke | 98-543 | 0.177 | 0.289 | −0.45 | −1.04054 | −0.21 | NA |
| Duke | 99-692 | −0.44 | −1 | 0.309 | 2.268985 | 0.033 | NA |
| Duke | 98-657 | 0.09 | −0.79 | −0.25 | 0.418497 | −0.14 | NA |
| Duke | 99-440 | 0.002 | 0.375 | −0.97 | −1.77929 | −0.08 | NA |
| Duke | 99-728 | −0.71 | 0.397 | 1.298 | −1.0632 | 0.49 | NA |
| Duke | 98-1146 | −0.6 | −0.16 | −0.23 | 0.628469 | 0.025 | NA |
| Duke | 98-771 | −0.57 | −1.63 | −0.4 | 1.076996 | −0.87 | NA |
| Duke | 98-1216 | 0.125 | −0.13 | 0.473 | 1.038565 | 0 | NA |
| Duke | 98-1014 | 0.675 | −0.13 | 0.848 | −3.08602 | −0.38 | NA |
| Duke | 99-830 | −0.62 | 1.021 | −2.08 | −2.9008 | 0.679 | NA |
| Duke | 00-11 | −0.59 | 0.387 | −0.15 | −1.5186 | 0.464 | NA |
| Duke | 98-152 | −0.29 | 0.172 | −0.58 | −1.23578 | −0.15 | NA |
| Duke | 98-1293 | −0.56 | 0.084 | −0.55 | −0.19295 | −0.59 | NA |
| Duke | 98-1296 | 0.707 | 0.213 | −0.56 | −0.73828 | −0.04 | NA |
| Duke | 98-375 | −0.59 | −0.52 | 0.208 | 0.32386 | −0.66 | NA |
| Duke | 98-967 | −1.1 | −1.55 | 0.376 | 0.409321 | −0.77 | NA |
| Duke | 99-1017 | −0.9 | −0.89 | −0.6 | 1.164087 | −1.08 | NA |
| Duke | 00-315 | 0.575 | 0.103 | 0.661 | −1.00921 | −0.62 | NA |
| Duke | 00-151 | −0.24 | −1.11 | 0.261 | −0.05388 | −0.18 | NA |
| Duke | 99-1067 | 0.011 | 0.166 | −0.18 | −1.21294 | 0.371 | NA |
| Duke | 99-301 | 0.036 | −0.76 | −0.3 | 0.619684 | −0.77 | NA |
| Duke | 99-137 | 0.615 | 0.134 | 2.151 | 0 | 0.178 | NA |
| Duke | 98-1063 | 0.004 | 0.235 | −0.31 | −0.43837 | −0.05 | NA |
| Duke | 98-343 | −0.29 | −0.12 | 0.268 | 0.910324 | −0.24 | NA |
| Duke | 98-186 | −1.14 | −0.3 | −0.42 | −2.09628 | 0.332 | NA |
| Duke | 98-691 | −0.38 | 0.462 | 1.377 | −1.03896 | −0.25 | NA |
| Duke | 98-723 | 0.763 | 0.369 | −0.65 | −1.04263 | −0.12 | NA |
| Duke | 98-197 | −0.13 | −0.81 | 0.226 | 1.377702 | 0.758 | NA |
| Duke | 98-828 | 0.379 | 0.078 | −0.37 | −2.29122 | 0.596 | NA |
| Duke | 97-1027 | 0.587 | 0.117 | −0.47 | 0.26364 | −0.37 | NA |
| Duke | 00-327 | 0.039 | −1.09 | −0.4 | 1.075552 | −0.05 | NA |
| Duke | 98-438 | 0.086 | −0.45 | 0.196 | 1.770386 | 0.458 | NA |
| Duke | 98-1277 | 0.202 | 0.742 | −0.91 | −0.4672 | 0.065 | NA |
| Duke | 00-703 | −0.22 | −0.7 | 0.45 | 1.347204 | 0.189 | NA |
| Duke | 00-440 | 0.094 | 0.399 | −1.22 | −1.85514 | 0.327 | NA |
| Duke | 98-956 | 0.6 | 0.672 | 0.077 | 0.955643 | −0.29 | NA |
| Duke | 00-909 | −0.92 | −1.21 | 1.001 | 0.928347 | −0.68 | NA |
| Duke | 97-666 | 0 | −0.78 | 0.099 | 1.151266 | −0.11 | NA |
| Duke | 97-608 | 0.514 | −0 | −0.12 | 0.491203 | −0.03 | NA |
| Duke | 97-829 | 0.57 | 0.38 | −0.34 | −1.08055 | 0.042 | NA |
| Duke | 00-550 | −0.54 | 0.311 | −1.02 | 0.520247 | 0.063 | NA |
| Duke | 99-706 | −0.07 | 0.294 | 0.035 | −1.19852 | 0.79 | NA |
| Duke | 98-417 | 1.338 | 0.684 | −0.41 | −1.26557 | −0.14 | NA |
| Duke | 96-264 | 0.463 | −0.53 | 0.362 | 2.249927 | 0.436 | NA |
| Duke | 97-792 | 0.425 | −0.33 | −0.03 | −0.55191 | −1.11 | NA |
| Duke | 96-353 | 0.025 | 0.262 | 0.263 | −1.21505 | −0.28 | NA |
| Duke | 00-145 | −0.81 | −0.35 | 0.796 | 0.719545 | 0.412 | NA |
| Duke | 00-253 | −0.11 | −0.06 | −1.49 | −0.31781 | 1.3 | NA |
| Duke | 00-334 | −1.06 | −0.62 | 0.812 | 1.071737 | 0.283 | NA |
| Duke | 00-398 | −0.33 | 1.207 | 0.392 | −0.67666 | 0.138 | NA |
| Duke | 00-452 | 0.437 | 0.693 | −0.63 | 0.567359 | 0.572 | NA |
| Duke | 00-479 | 0.567 | 0.313 | 0.472 | 0.592302 | 0.264 | NA |
| Duke | 00-827 | −0.02 | −0.82 | −1.23 | 0.707033 | 0.379 | NA |
| Duke | 00-941 | −0.58 | 0.199 | 0.708 | −0.57326 | 0.513 | NA |
| Duke | 00-1059 | −0.03 | 0.097 | 0.796 | −1.41237 | 0.323 | NA |
| Duke | 00-1072 | −0.34 | −0.59 | 0.534 | 1.638961 | 0.534 | NA |
| Duke | 00-1082 | −0.49 | −0.64 | 0.255 | 1.541737 | 0.407 | NA |
| Duke | 01-181 | 0.08 | −0.79 | 1.534 | 2.024381 | 0.029 | NA |
| Duke | 01-189 | 0.03 | 0.288 | 0.692 | 0.656979 | −0.2 | NA |
| Duke | 01-236 | −0.76 | 0.163 | −1.95 | −2.66171 | 0.859 | NA |
| Duke | 01-331 | 0.355 | 0.891 | 0.765 | 0.300173 | 0.497 | NA |
| Duke | 01-646 | 0.393 | −0.12 | −0.29 | 1.357886 | 0.03 | NA |
| Duke | 01-284 | −0.2 | 0.277 | −1.2 | −0.59169 | 0.1 | NA |
| Duke | 01-369 | −0.73 | −1.44 | −0.24 | 2.351711 | −0.1 | NA |
| Duke | 01-424 | 0.917 | 0 | −0.78 | −0.19251 | 0.634 | NA |
| Duke | 01-534 | 0.244 | −0.26 | −0.36 | −0.09865 | 0.267 | NA |
| Duke | 01-139 | −0.24 | 1.274 | −0.13 | 0.893 | 0.38 | NA |
| Duke | 97-930 | 0.025 | 1.005 | 0 | −1.9082 | 0.318 | NA |
| MI06 | LS-1 | 0.493 | −0.53 | −0.99 | 1.296624 | 0.842 | NA |
| MI06 | LS-10 | −0.95 | 0.537 | −2.47 | −0.24335 | 0.762 | NA |
| MI06 | LS-100 | 0.322 | 0.132 | −1.93 | 0.409942 | −0.21 | NA |
| MI06 | LS-101 | −0.15 | 0.088 | −1.92 | −0.83692 | −0.1 | NA |
| MI06 | LS-102 | −0.71 | −0.18 | −0.65 | −0.91093 | −0.5 | NA |
| MI06 | LS-103 | 0.042 | 0.674 | 2.98 | 0.019644 | 0.142 | NA |
| MI06 | LS-104 | 0.201 | 0.07 | 0.308 | −0.41521 | −0.28 | NA |
| MI06 | LS-105 | 0.341 | −0 | 0.372 | −0.09948 | 1.208 | NA |
| MI06 | LS-106 | 0.444 | −0.17 | 0.63 | −0.12755 | 0.79 | NA |
| MI06 | LS-107 | 1.104 | 0.483 | 2.876 | −0.25794 | 0.168 | NA |
| MI06 | LS-108 | 0.211 | −0.29 | 0.69 | 0.769267 | 0.034 | NA |
| MI06 | LS-109 | 0.876 | 0.3 | 0.398 | −1.28195 | 0.076 | NA |
| MI06 | LS-111 | 0.995 | 0.52 | 1.328 | −0.56429 | −0.06 | NA |
| MI06 | LS-113 | −0.1 | −0.12 | −0.63 | 0.653446 | −0.16 | NA |
| MI06 | LS-114 | 1 | −0.24 | 1.616 | 0.442505 | 0.003 | NA |
| MI06 | LS-115 | −0.22 | −0.48 | 0.72 | −0.384 | 1.195 | NA |
| MI06 | LS-116 | 0.233 | −0.35 | −2.91 | −0.33351 | −0.91 | NA |
| MI06 | LS-117 | 0.871 | 0.076 | −0.99 | 0.606582 | 0.345 | NA |
| MI06 | LS-118 | −0.19 | 0.131 | −0.01 | −0.99161 | 0.61 | NA |
| MI06 | LS-119 | 1.023 | 0.338 | 0.269 | 0.122699 | 0.108 | NA |
| MI06 | LS-12 | −0.42 | 0.153 | −2.89 | 0.209154 | 0.6 | NA |
| MI06 | LS-120 | 0.248 | −0.11 | −0.36 | 0.735172 | −0.17 | NA |
| MI06 | LS-121 | −0.1 | 1.007 | 1.128 | −1.43229 | 0.007 | NA |
| MI06 | LS-122 | 0.316 | 0.468 | −0.83 | −0.35644 | 0.176 | NA |
| MI06 | LS-123 | 0.617 | −0.4 | 0.986 | 1.717957 | 0.525 | NA |
| MI06 | LS-124 | 0.446 | −0.12 | 0.129 | 0.964845 | 0.335 | NA |
| MI06 | LS-125 | 0.659 | 0.245 | 0.77 | 1.668951 | 1.246 | NA |
| MI06 | LS-126 | −0.33 | 0.214 | 0.268 | 0.674554 | 0.466 | NA |
| MI06 | LS-127 | 0.087 | 0.119 | 1.051 | 1.210976 | 0.506 | NA |
| MI06 | LS-128 | −0.44 | −0.15 | 1.201 | 1.070839 | 0.709 | NA |
| MI06 | LS-129 | −0.11 | 0.36 | −1.65 | −0.85793 | −0.18 | NA |
| MI06 | LS-13 | −0.72 | 0.219 | −2.85 | −0.92294 | 0.44 | NA |
| MI06 | LS-130 | 0.515 | −0.19 | 0.934 | 1.500999 | 0.558 | NA |
| MI06 | LS-131 | 0.133 | 0.833 | 1.062 | 0.593799 | 0.038 | NA |
| MI06 | LS-132 | −1 | −0.19 | −0.36 | 0.290651 | 1.09 | NA |
| MI06 | LS-133 | −0.05 | 1.143 | 0.803 | 0.523098 | 0.83 | NA |
| MI06 | LS-134 | −0.32 | 0.151 | −1.93 | −0.21195 | 0.859 | NA |
| MI06 | LS-135 | 0.115 | −0.33 | −0.71 | 0.508895 | 1.363 | NA |
| MI06 | LS-136 | −0.01 | −0.35 | −1.89 | 1.280201 | 0.027 | NA |
| MI06 | LS-138 | −0.22 | −0.12 | 1.389 | −1.24585 | 0.12 | NA |
| MI06 | LS-139 | 0.852 | 0.315 | 0.572 | 0.58637 | 0.749 | NA |
| MI06 | LS-14 | 0.081 | −0.1 | −0.36 | −0.44674 | 0.333 | NA |
| MI06 | LS-140 | −0.49 | 0.229 | −0.47 | 1.010209 | −0.1 | NA |
| MI06 | LS-15 | 0.508 | −0.38 | −2.97 | −0.41425 | 0.584 | NA |
| MI06 | LS-16 | −0.89 | 0.179 | −2.59 | 1.357967 | 0.433 | NA |
| MI06 | LS-17 | −0.51 | −0.14 | −2.29 | −1.12395 | 1.091 | NA |
| MI06 | LS-18 | −0.87 | 0.59 | −1.83 | −1.94439 | −0.26 | NA |
| MI06 | LS-19 | 0.319 | 0.058 | −3.1 | 0.422529 | −1 | NA |
| MI06 | LS-2 | 0.406 | 0.84 | −2.06 | 0.25877 | 0.726 | NA |
| MI06 | LS-20 | 0.294 | 0.292 | −0.06 | 0.087387 | −0.43 | NA |
| MI06 | LS-21 | 0.39 | −0.21 | −1.5 | 0.200962 | −0.1 | NA |
| MI06 | LS-22 | 0.5 | −0.21 | −2.61 | 1.644532 | −0.31 | NA |
| MI06 | LS-23 | 0.261 | −0.77 | −0.63 | 1.075569 | −0.14 | NA |
| MI06 | LS-24 | −0.28 | 0.647 | 0.16 | −2.1436 | 0.168 | NA |
| MI06 | LS-25 | 0.582 | −0.72 | −1.92 | 1.072402 | −1.11 | NA |
| MI06 | LS-26 | −0.12 | 0.295 | −0.74 | 0.762505 | 0.482 | NA |
| MI06 | LS-27 | −0.38 | 0.099 | 0.758 | −0.86887 | 0.051 | NA |
| MI06 | LS-28 | −0.67 | 0.066 | −3.56 | 0.272814 | −0.69 | NA |
| MI06 | LS-29 | 0.56 | 0.197 | 0.316 | 0.117799 | −0.01 | NA |
| MI06 | LS-30 | −0.18 | 0.266 | −0.02 | −0.18008 | 0.264 | NA |
| MI06 | LS-31 | 0.438 | −0.48 | 0.161 | 1.041374 | −0.25 | NA |
| MI06 | LS-32 | 0.743 | −0.23 | −2.38 | −0.95227 | 1.624 | NA |
| MI06 | LS-33 | 0.007 | −0.4 | 0.634 | 0.212463 | 0.542 | NA |
| MI06 | LS-34 | −0.46 | 0.584 | −1.43 | −1.1083 | 0.485 | NA |
| MI06 | LS-35 | 0.491 | 0.594 | 0.279 | −1.64348 | 0.693 | NA |
| MI06 | LS-36 | −0.2 | −0.91 | −0.37 | −0.53383 | 0.248 | NA |
| MI06 | LS-37 | 0.831 | 0.313 | 0.396 | −0.36098 | 0.366 | NA |
| MI06 | LS-38 | 0.285 | −0.18 | −0.19 | 1.434433 | −0.27 | NA |
| MI06 | LS-39 | 0.909 | 0.443 | −2.03 | −1.33458 | −0.27 | NA |
| MI06 | LS-40 | −0.2 | −0.48 | −1.93 | 0.407861 | −0.48 | NA |
| MI06 | LS-41 | −0.31 | −0.32 | 0.006 | −0.80137 | −0.22 | NA |
| MI06 | LS-42 | −0.78 | −0.41 | 0.348 | −0.95396 | −0.6 | NA |
| MI06 | LS-43 | −0.04 | −0.54 | 0.243 | 0.512445 | −0.35 | NA |
| MI06 | LS-44 | −1.22 | −0.19 | −1.48 | −0.77617 | −1.2 | NA |
| MI06 | LS-45 | 0.59 | −0.4 | 0.269 | −1.10605 | −0.18 | NA |
| MI06 | LS-46 | −0.43 | −0.14 | −1.66 | 0.002708 | −0.51 | NA |
| MI06 | LS-47 | −0.48 | −0.2 | 0.219 | 0.366527 | −0.57 | NA |
| MI06 | LS-48 | −0.63 | 0.542 | 0.71 | −1.89818 | −0.43 | NA |
| MI06 | LS-49 | −0.64 | 0.112 | 1.213 | −0.36804 | −0.63 | NA |
| MI06 | LS-5 | −0.29 | 0.279 | −2.62 | −0.47766 | 1.497 | NA |
| MI06 | LS-50 | −0.75 | 0.572 | 0.454 | −2.21531 | 0.268 | NA |
| MI06 | LS-51 | −1.04 | −0.09 | −2.79 | 0.109888 | −0.61 | NA |
| MI06 | LS-52 | −0.97 | 0.135 | 0.457 | −0.28609 | 0.064 | NA |
| MI06 | LS-53 | −0.23 | −0.15 | −0.83 | 1.374901 | −0.02 | NA |
| MI06 | LS-54 | −0.17 | 0.499 | 0.918 | −1.03554 | −0.49 | NA |
| MI06 | LS-55 | 0.345 | 0.316 | 0.705 | −1.62197 | 0.112 | NA |
| MI06 | LS-56 | 0.126 | −0.11 | 0.5 | 0.899775 | −1.22 | NA |
| MI06 | LS-57 | 0.009 | −0.13 | −0.89 | −0.93807 | 1.129 | NA |
| MI06 | LS-58 | −0.3 | −0.65 | −1.25 | 1.746071 | −0.29 | NA |
| MI06 | LS-59 | 0.193 | 0.278 | −1.04 | 0.239382 | 0.06 | NA |
| MI06 | LS-6 | 0.1 | 0.366 | 0.884 | 0.343867 | −0.04 | NA |
| MI06 | LS-60 | 0.463 | −0.28 | 0.158 | −0.03737 | −0.57 | NA |
| MI06 | LS-61 | 0.463 | −0.18 | −2.27 | 0.132094 | −1.06 | NA |
| MI06 | LS-62 | 0.65 | 0.285 | 1.08 | −0.40381 | −0.04 | NA |
| MI06 | LS-63 | −1.43 | 0.813 | 0.353 | −0.596 | 0.4 | NA |
| MI06 | LS-64 | −0.9 | 0.351 | 0.894 | 0.083324 | 0.059 | NA |
| MI06 | LS-65 | −0.23 | −0.29 | −0.44 | −0.53308 | −0.96 | NA |
| MI06 | LS-66 | 0.38 | 0.272 | −0.43 | −0.10854 | −0.22 | NA |
| MI06 | LS-67 | −0.62 | −0.25 | 0.213 | 0.16171 | −0.12 | NA |
| MI06 | LS-68 | 0.339 | −0.63 | −3.15 | 1.145948 | −0.2 | NA |
| MI06 | LS-69 | 0.51 | −0.18 | −0.31 | −1.18423 | 0.01 | NA |
| MI06 | LS-70 | −0.84 | 0.53 | −0.29 | −0.52718 | 0.395 | NA |
| MI06 | LS-71 | −0.66 | 0.001 | −3 | 1.031878 | −0.55 | NA |
| MI06 | LS-72 | −0.99 | 0.326 | 0.131 | −0.80031 | 0.519 | NA |
| MI06 | LS-73 | −0.13 | −0.4 | −0.38 | −0.74013 | −1.22 | NA |
| MI06 | LS-74 | 0.005 | −0.52 | 0.319 | 0.857927 | −0.5 | NA |
| MI06 | LS-75 | 0.424 | −0.21 | −1.45 | 0.548173 | 0.134 | NA |
| MI06 | LS-77 | −0.14 | −0.27 | 1.137 | −0.17323 | −0.14 | NA |
| MI06 | LS-78 | −1.32 | −0.25 | 0.026 | −2.36656 | −0.66 | NA |
| MI06 | LS-79 | 0.588 | −0.06 | 0.053 | 0.132241 | −0.08 | NA |
| MI06 | LS-8 | 0.446 | −0.7 | −1.38 | −0.00271 | −0.29 | NA |
| MI06 | LS-80 | 0.595 | −0.09 | 0.645 | 0.339086 | 0.101 | NA |
| MI06 | LS-81 | −0.18 | −0.19 | 0.146 | −0.66778 | −0.48 | NA |
| MI06 | LS-82 | −0.49 | 0.212 | 1.427 | −0.33322 | −0.85 | NA |
| MI06 | LS-83 | −2.33 | −0.49 | −0.49 | −0.38039 | −0.24 | NA |
| MI06 | LS-85 | −0.86 | −1.16 | −0.41 | 1.258565 | −0.25 | NA |
| MI06 | LS-86 | −0.13 | 0.259 | −2.53 | 0.399665 | −0.09 | NA |
| MI06 | LS-87 | 0.307 | 0.1 | 0.599 | 0.022488 | −0.03 | NA |
| MI06 | LS-88 | −0.08 | −0.5 | 0.636 | −0.46251 | −0.22 | NA |
| MI06 | LS-89 | −0.12 | 0.261 | 0.8 | 0.094157 | 0.182 | NA |
| MI06 | LS-9 | 0.186 | 1.112 | −0.69 | −0.56716 | 0.89 | NA |
| MI06 | LS-90 | −0.17 | −0.08 | −0.43 | −0.72358 | 0.153 | NA |
| MI06 | LS-91 | 0.615 | 0.815 | 1.272 | 0.169645 | −0.68 | NA |
| MI06 | LS-92 | −1 | 0.003 | −0.3 | −0.40104 | −0.06 | NA |
| MI06 | LS-94 | 0.86 | 0.532 | 0.468 | 0.270417 | −0.19 | NA |
| MI06 | LS-95 | 0.391 | 0.409 | 0.762 | −1.3824 | 0.167 | NA |
| MI06 | LS-96 | −0.42 | −0.2 | 1.3 | 0.215918 | −0.17 | NA |
| MI06 | LS-97 | −0.21 | 0.503 | −0.74 | −0.63622 | −0 | NA |
| MI06 | LS-98 | 0.169 | −0.53 | 0.621 | −0.77162 | −0.65 | NA |
| MI06 | LS-99 | 0.192 | −0.45 | 0.318 | 1.146439 | 0.375 | NA |
| AD1 | Sample_A1 | 0.832 | 0.228 | −0.13 | −0.04932 | NA | NA |
| AD1 | Sample_A2 | 1.426 | 0.14 | NA | −0.1227 | NA | NA |
| AD1 | Sample_A3 | 0.976 | −0.03 | −0.26 | −0.13327 | NA | NA |
| AD1 | Sample_A4 | 0.195 | 0.03 | 0.082 | 0.11901 | NA | NA |
| AD1 | Sample_A5 | 0.341 | 0.439 | −0.21 | −0.77958 | NA | NA |
| AD1 | Sample_A6 | 0.044 | −0.41 | −0.04 | 0.84331 | NA | NA |
| AD1 | Sample_A8 | −0.08 | −0.06 | NA | 0.054037 | NA | NA |
| AD1 | Sample_A9 | 0.143 | −0.2 | 0.035 | −0.25414 | NA | NA |
| AD1 | Sample_A10 | −0.14 | 0.065 | −0.12 | −0.01695 | NA | NA |
| AD1 | Sample_A11 | −0.29 | −0.2 | 0.032 | 0.242846 | NA | NA |
| AD1 | Sample_A12 | −0.25 | 0.153 | −0.09 | −0.64062 | NA | NA |
| AD1 | Sample_A13 | 0.056 | −0.1 | −0.06 | 1.151475 | NA | NA |
| AD1 | Sample_A14 | 0.611 | 0.01 | 0.054 | 0.708476 | NA | NA |
| AD1 | Sample_A15 | −0.81 | 0.298 | −0.22 | 0.090488 | NA | NA |
| AD1 | Sample_A16 | −0.33 | −0.12 | −0.05 | 0.461766 | NA | NA |
| AD1 | Sample_A17 | −0.44 | −0.45 | 0.056 | 0.016947 | NA | NA |
| AD1 | Sample_A18 | 0.01 | 0.234 | NA | 0.436069 | NA | NA |
| AD1 | Sample_A19 | 2.014 | 0.045 | −0.2 | −0.55061 | NA | NA |
| AD1 | Sample_A20 | −0.82 | −0.13 | 0.186 | 1.82684 | NA | NA |
| AD1 | Sample_A21 | −0.88 | −0.29 | 0.063 | 1.885393 | NA | NA |
| AD1 | Sample_A22 | 0.205 | −0.07 | 0.028 | 0.159572 | NA | NA |
| AD1 | Sample_A23 | −0.57 | 0.174 | −0.16 | −0.13016 | NA | NA |
| AD1 | Sample_A24 | −1.38 | −0.11 | 0.007 | 0.800435 | NA | NA |
| AD1 | Sample_A25 | 0.256 | 0.074 | −0.01 | 0.093631 | NA | NA |
| AD1 | Sample_A26 | 1.296 | −0.07 | −0.27 | 0.346722 | NA | NA |
| AD1 | Sample_A27 | 0.769 | 0.374 | 0.109 | −0.17389 | NA | NA |
| AD1 | Sample_A28 | 0.03 | 0.553 | 0.263 | 0.480807 | NA | NA |
| AD1 | Sample_A29 | −0.31 | 0.167 | NA | −0.34642 | NA | NA |
| AD1 | Sample_A30 | 1.458 | −0.34 | −0.03 | −0.59704 | NA | NA |
| AD1 | Sample_A31 | 0.017 | −0.62 | NA | 0.437364 | NA | NA |
| AD1 | Sample_A32 | −0.68 | 0.83 | 0.177 | −1.00999 | NA | NA |
| AD1 | Sample_A33 | −0.2 | −0.58 | −0.04 | −0.19166 | NA | NA |
| AD1 | Sample_A34 | 0.247 | 0.063 | 0.052 | −0.07482 | NA | NA |
| AD1 | Sample_A35 | −0.04 | −0.15 | NA | −0.56454 | NA | NA |
| AD1 | Sample_A36 | 0.424 | −0.28 | −0.01 | 0.276731 | NA | NA |
| AD1 | Sample_A37 | −0.63 | 0.273 | 0.025 | −0.15683 | NA | NA |
| AD1 | Sample_A38 | −0.05 | 0.042 | NA | 0.612486 | NA | NA |
| AD1 | Sample_A39 | −0.01 | −0.83 | 0.136 | −0.24803 | NA | NA |
| AD1 | Sample_A40 | 1.197 | −0.11 | −0.26 | 0.979008 | NA | NA |
| AD1 | Sample_A41 | 0.982 | −0.09 | 0.102 | −0.1643 | NA | NA |
| AD1 | Sample_A42 | −0.82 | −0.05 | 0.044 | −0.52691 | NA | NA |
| AD1 | Sample_A43 | −0.26 | 0.229 | NA | −0.38756 | NA | NA |
| AD1 | Sample_A44 | −0.56 | −0.01 | −0.03 | 0.54584 | NA | NA |
| AD1 | Sample_A45 | −0.62 | 0.355 | NA | −0.13693 | NA | NA |
| AD1 | Sample_A46 | −0.25 | 0.415 | NA | −0.44353 | NA | NA |
| AD1 | Sample_A47 | 0.251 | −0.32 | 0.072 | 1.489913 | NA | NA |
| AD1 | Sample_A48 | 0.107 | 0.526 | −0.13 | −0.49501 | NA | NA |
| AD1 | Sample_A49 | −0.31 | 0.267 | 0.139 | 0.400408 | NA | NA |
| SQ2 | Sample_N1 | 1.618 | 0.562 | 0.137 | 0.027884 | NA | NA |
| SQ2 | Sample_N2 | 0.536 | −0.05 | 0.108 | 0.032999 | NA | NA |
| SQ2 | Sample_N3 | 0.454 | 0.102 | 0.094 | −1.02194 | NA | NA |
| SQ2 | Sample_N4 | 0.187 | −0.1 | 0.055 | 0 | NA | NA |
| SQ2 | Sample_N5 | 0.081 | −0.02 | 0.238 | 0.337902 | NA | NA |
| SQ2 | Sample_N6 | 0.17 | 0.077 | 0.117 | −0.12433 | NA | NA |
| SQ2 | Sample_N7 | −0.06 | −0.07 | 0.049 | 0.190636 | NA | NA |
| SQ2 | Sample_N8 | 0.852 | −0.02 | 0.036 | −0.01966 | NA | NA |
| SQ2 | Sample_N9 | NA | 0.059 | 0.023 | 0.03012 | NA | NA |
| SQ2 | Sample_N10 | 0.151 | −0.3 | 0.069 | −0.0645 | NA | NA |
| SQ2 | Sample_N11 | NA | −0.3 | −0.12 | 0.325634 | NA | NA |
| SQ2 | Sample_N12 | −0.3 | 0.063 | −0.06 | 0.049238 | NA | NA |
| SQ2 | Sample_N13 | NA | 0.264 | 0.177 | −0.04365 | NA | NA |
| SQ2 | Sample_N14 | −0.56 | 0.055 | 0.354 | 0.080067 | NA | NA |
| SQ2 | Sample_N15 | −0.86 | 0.176 | 0.029 | −0.01679 | NA | NA |
| SQ2 | Sample_N16 | −0.06 | 0.244 | −0 | 0.134597 | NA | NA |
| SQ2 | Sample_N17 | −0.25 | −0.22 | −0.07 | −0.14612 | NA | NA |
| SQ2 | Sample_N18 | 0.461 | 0.378 | −0.07 | 0.027353 | NA | NA |
| SQ2 | Sample_N19 | 0.862 | 0.042 | 0.066 | −0.10602 | NA | NA |
| SQ2 | Sample_N20 | 0.509 | 0.167 | 0.048 | 0.060212 | NA | NA |
| SQ2 | Sample_N21 | −0.71 | 0.4 | −0.22 | −0.26515 | NA | NA |
| SQ2 | Sample_N22 | −0.76 | −0.27 | −0.04 | −0.06655 | NA | NA |
| SQ2 | Sample_N23 | 0.971 | −0.71 | −0.12 | −0.11278 | NA | NA |
| SQ2 | Sample_N24 | −1.3 | −0.02 | 0.088 | −0.09691 | NA | NA |
| SQ2 | Sample_N25 | −2.04 | −0.14 | −0.07 | −0.08164 | NA | NA |
| SQ2 | Sample_N26 | 0.101 | 0.322 | −0.08 | −0.04549 | NA | NA |
| SQ2 | Sample_N27 | −0.32 | −0.25 | −0.07 | −0.06555 | NA | NA |
| SQ2 | Sample_N28 | −0.69 | 0.245 | 0.018 | 0.020244 | NA | NA |
| SQ2 | Sample_N29 | 0.352 | 0 | −0.06 | 0.008545 | NA | NA |
| SQ2 | Sample_N30 | −0.22 | −0.04 | 0.12 | 0.175576 | NA | NA |
| SQ2 | Sample_N31 | −0.99 | 0.059 | 0.157 | 0.012825 | NA | NA |
| SQ2 | Sample_N32 | 0.902 | −0.18 | 0.078 | −0.01264 | NA | NA |
| SQ2 | Sample_R1 | 1.003 | −0.17 | 0 | −0.27674 | NA | NA |
| SQ2 | Sample_R2 | 0.196 | 0.182 | −0.02 | −0.19898 | NA | NA |
| SQ2 | Sample_R3 | 0.604 | −0.13 | −0.05 | 0.059296 | NA | NA |
| SQ2 | Sample_R4 | −0.59 | 0.179 | −0.26 | −0.16235 | NA | NA |
| SQ2 | Sample_R5 | −0.8 | −0.12 | 0.215 | −0.09589 | NA | NA |
| SQ2 | Sample_R6 | 4.72 | −0.04 | 0.042 | −0.30542 | NA | NA |
| SQ2 | Sample_R7 | −0.37 | 0.008 | 0.052 | −0.11855 | NA | NA |
| SQ2 | Sample_R8 | −1.08 | 0.187 | 0.086 | 0.071134 | NA | NA |
| SQ2 | Sample_R9 | 1.148 | 0.396 | 0.086 | 0.123135 | NA | NA |
| SQ2 | Sample_R10 | 0.276 | 0.789 | −0.11 | −0.05432 | NA | NA |
| SQ2 | Sample_R11 | 0.011 | 0.433 | −0.04 | 0.096925 | NA | NA |
| SQ2 | Sample_R12 | −0.63 | 0.057 | 0.044 | −0.04402 | NA | NA |
| SQ2 | Sample_R13 | −0.97 | 0.158 | 0.047 | −0.08769 | NA | NA |
| SQ2 | Sample_R14 | −0.01 | 0.167 | −0.03 | 0.263372 | NA | NA |
| SQ2 | Sample_R15 | 0.515 | 0.216 | 0.153 | −0.00754 | NA | NA |
| SQ2 | Sample_R16 | 4.72 | −0.23 | −0.06 | −0.13583 | NA | NA |
| SQ2 | Sample_R17 | 0.391 | −0.03 | 0.058 | 0.071606 | NA | NA |
| SQ2 | Sample_R18 | −0.14 | 0.226 | −0.04 | −0.01465 | NA | NA |
| SQ2 | Sample_R19 | −1.05 | −0.25 | −0.01 | −0.25237 | NA | NA |
| SQ2 | Sample_S1 | −0.23 | −0.17 | −0.51 | 0.684999 | NA | NA |
| SQ2 | Sample_S2 | −0.32 | −0.16 | −0.6 | 0.883382 | NA | NA |
| SQ2 | Sample_S3 | −0.51 | −0.14 | −0.34 | 0.264022 | NA | NA |
| SQ2 | Sample_S4 | 0.65 | −0.25 | −0.64 | 1.57778 | NA | NA |
| SQ2 | Sample_S5 | 0.024 | −0.27 | −0.61 | 0.35091 | NA | NA |
| SQ2 | Sample_S6 | −0.29 | −0.21 | −0.65 | 1.336932 | NA | NA |
| SQ2 | Sample_S7 | −0.27 | −0.1 | −0.36 | 0.871311 | NA | NA |
| SQ2 | Sample_S8 | 0.977 | 0.079 | −0.72 | 1.116645 | NA | NA |
| LuMayo | 40430 | −0.07 | 0.007 | 0.092 | 0.121905 | −0.18 | NA |
| LuMayo | 41923 | 0.551 | −0.01 | −0.04 | −0.61129 | −0.56 | NA |
| LuMayo | 41932 | 0.008 | 0.437 | 0.589 | 0.98936 | −0.25 | NA |
| LuMayo | 42081 | −0.45 | 0.746 | 0.406 | −1.90906 | 0.059 | NA |
| LuMayo | 42613 | −0.66 | −0.61 | −0.23 | 1.400512 | 0.706 | NA |
| LuMayo | 42616 | −0.19 | −0.5 | −0.34 | 0.594914 | 0.359 | NA |
| LuMayo | 44656 | 0.14 | 0.451 | −0.04 | 0.113992 | −0.26 | NA |
| LuMayo | 44661 | −0.52 | −0.44 | 0.544 | −0.23019 | −0.13 | NA |
| LuMayo | 44680 | −0.19 | 0.479 | −0.24 | 0.74732 | 0.013 | NA |
| LuMayo | 44693 | −0.01 | −0.25 | −0.62 | 1.451466 | −0.02 | NA |
| LuMayo | 48521 | 0.52 | −0.59 | 0.273 | 0.466128 | −0.01 | NA |
| LuMayo | 48536 | −0.12 | 0.345 | 0.662 | −0.5179 | 0.503 | NA |
| LuMayo | 48549 | 0.287 | −0.33 | −0.33 | 1.514134 | 0.058 | NA |
| LuMayo | 48556 | 0.149 | −0.14 | −0.22 | −0.70007 | 0.195 | NA |
| LuMayo | 57774 | 0.687 | 0.189 | 0.021 | −0.68184 | 0.379 | NA |
| LuMayo | 76981 | 0.19 | −0.52 | 0.352 | −0.30926 | 0.178 | NA |
| LuMayo | 86011 | 0.315 | 0.686 | 0.442 | −0.19706 | −0.29 | NA |
| LuMayo | 86043 | −0.22 | 0.418 | −0.02 | −0.11399 | −0.31 | NA |
| LuWashU | 3196 | 0.109 | 0.989 | 0.367 | −0.21985 | 0.269 | NA |
| LuWashU | 3197 | −0.47 | 0.211 | −0.1 | 0.381697 | −0.45 | NA |
| LuWashU | 3200 | 0.285 | 0.525 | 0.517 | −2.38304 | 0.424 | NA |
| LuWashU | 3202 | −0.3 | −1 | 0.409 | 0.585283 | 0.44 | NA |
| LuWashU | 3205 | −0.17 | 0.222 | 0.636 | −0.37989 | 0.448 | NA |
| LuWashU | 3210 | 1.353 | −1 | 0.829 | 1.759558 | 0.632 | NA |
| LuWashU | 3211 | 0.619 | 0.978 | 0.649 | 0.259898 | 0.823 | NA |
| LuWashU | 3213 | 0.264 | −0.01 | −0.02 | −1.67816 | −0.02 | NA |
| LuWashU | 3218 | 1.865 | −1 | 1.636 | −0.43249 | 1.375 | NA |
| LuWashU | 3223 | −0.41 | −0.93 | −0.13 | 0.389914 | −0.18 | NA |
| LuWashU | 3226 | 1.215 | −0.6 | 0.368 | 0.245982 | 0.82 | NA |
| LuWashU | 3227 | −0.43 | −0.14 | −0.52 | 1.558145 | −0.44 | NA |
| LuWashU | 3229 | 0.19 | −0.78 | −0.44 | 0.124655 | −0.04 | NA |
| LuWashU | 3230 | 1.075 | 0.119 | 0.625 | 1.242203 | 0.802 | NA |
| LuWashU | 3198 | −0.59 | 0.968 | −0.07 | −0.13048 | 0.171 | NA |
| LuWashU | 3199 | −0.51 | −0.29 | −0.72 | −0.25085 | −0.16 | NA |
| LuWashU | 3201 | −0.11 | 0.247 | 0.206 | −0.6536 | 0.251 | NA |
| LuWashU | 3203 | −0.21 | 0.007 | −0.12 | 0.571897 | −0.06 | NA |
| LuWashU | 3204 | −0.02 | 0.269 | −0.32 | 0.496371 | −0.23 | NA |
| LuWashU | 3206 | −0.05 | 0.319 | −0.12 | −0.37682 | −0.35 | NA |
| LuWashU | 3208 | −0.04 | −0.02 | −0.54 | 1.267476 | −0.43 | NA |
| LuWashU | 3209 | 0.792 | 1.315 | 1.375 | 2.516684 | 1.252 | NA |
| LuWashU | 3214 | 0.122 | −0.56 | −0.29 | −1.36801 | 0.009 | NA |
| LuWashU | 3215 | 0.296 | −0.61 | −0.29 | 0.600525 | −0.31 | NA |
| LuWashU | 3216 | −1.14 | −0.3 | 0.285 | 0.64946 | −0.01 | NA |
| LuWashU | 3217 | −0 | −0.28 | 0.278 | 0.402338 | 0.126 | NA |
| LuWashU | 3220 | 0.005 | −0.65 | 0.022 | −0.16376 | −0.03 | NA |
| LuWashU | 3221 | 0.874 | −0.06 | −0.23 | −1.12223 | −0.19 | NA |
| LuWashU | 3224 | 0.07 | −0.32 | −0.6 | −0.6894 | −0.22 | NA |
| LuWashU | 3225 | 0.042 | 0.507 | −0.16 | −1.41348 | −0.03 | NA |
| LuWashU | 3228 | −0.08 | 0.655 | 0.178 | −0.12465 | 0.123 | NA |
| LuWashU | 3231 | −0.3 | 0.807 | −0.52 | 0.804761 | −0.45 | NA |

TABLE 3
Validation Datasets

| | Patients | | | |
|---|---|---|---|---|
| | (Classified/ | Hazard Ratio | | |
| Dataset Name | Total) | (95% C.I.) | P-Value | Reference |
| Training Dataset | 147/147 | 4.8 (2.4-9.5) | 9.8 × 10⁻⁶ | Lau et al. |
| Cross Validation | 147/147 | 2.5 (1.4-4.8) | 0.0035 | Lau et al. |
| Duke | 71/91 | 3.3 (1.6-6.9) | 0.002 | Potti et al. |
| Larsen Squamous | 59/59 | 2.2 (0.7-6.6) | 0.16 | Larsen et al. |
| MI06 Validation | 100/130 | 1.4 (0.9-3.5) | 0.08 | Raponi et al. |
| Larsen | 48/48 | 2.9 (1.2-7.0) | 0.02 | Larson et al. |
| Adenocarcinoma | | | | |
| Pooled (All | 493/589 | 1.6 (1.2-2.2) | 7.6 × 10⁻⁴ | Multiple |
| Patients) | | | | |
| Pooled (Stage I | 345/409 | 1.5 (1.1-2.2) | 0.022 | Multiple |
| Patients) | | | | |

TABLE 4
Permutation Analysis

| | | Dataset | | |
|---|---|---|---|---|
| | | Lau | Potti | Beer |
| 6 Gene | Total Permutations | 10,000,000 | 9,999,722 | 9,999,114 |
| Permu- | Missing Values | 0 | 278 | 886 |
| tations | Permutations(p < | 1,640,991 | 452,083 | 1,136,375 |
| | 0.05) | | | |
| | % of Permutations(p < | 16.41 | 4.52 | 11.36 |
| | 0.05) | | | |
| | mSD chi-squared | 31.4 | 9.8 | 6.4 |
| | value | | | |
| | Permutations(p < | 114 | 13,521 | 434,784 |
| | mSD) | | | |
| | % of Permutations(p < | 1.14E−03 | 0.14 | 4.35 |
| | mSD) | | | |

| | | Dataset | |
|---|---|---|---|
| | | Raponi | Bhattacharjee |
| 6 Gene | Total Permutations | 9,999,676 | 9,999,621 |
| Permu- | Missing Values | 324 | 379 |
| tations | Permutations(p < | 480,422 | 906,509 |
| | 0.05) | | |
| | % of Permutations(p < | 4.80 | 9.07 |
| | 0.05) | | |
| | mSD chi-squared | 2.6 | 6.7 |
| | value | | |
| | Permutations(p < | 1,042,445 | 221,882 |
| | mSD) | | |
| | % of Permutations(p < | 10.42 | 2.22 |
| | mSD) | | |

TABLE 5

| Gene | | | | | | |
|---|---|---|---|---|---|---|
| ID | Gene Symbol | Total Subsets | Subsets p < 0.05 | Fraction Subsets p < 0.05 | Enrichment | P |
| 10 | CALCA | 530888 | 228926 | 0.431213363 | 2.6 | <2.2E−16 |
| 12 | CCR7 | 530559 | 221226 | 0.416967764 | 2.5 | <2.2E−16 |
| 99 | STX1A | 530389 | 215827 | 0.406922089 | 2.5 | <2.2E−16 |
| 13 | CCT3 | 531702 | 188951 | 0.355370113 | 2.2 | <2.2E−16 |
| 97 | SPRR1B | 531492 | 186510 | 0.350917794 | 2.1 | <2.2E−16 |
| 86 | SELP | 530971 | 182091 | 0.342939633 | 2.1 | <2.2E−16 |
| 71 | PAFAH1B3 | 532345 | 174229 | 0.327285877 | 2.0 | <2.2E−16 |
| 24 | CPE | 530091 | 163165 | 0.307805641 | 1.9 | <2.2E−16 |
| 112 | XRCC6 | 531083 | 150103 | 0.282635671 | 1.7 | <2.2E−16 |
| 43 | HIF1A | 531543 | 143440 | 0.269855872 | 1.6 | <2.2E−16 |
| 62 | MARCH6 | 530514 | 142543 | 0.268688479 | 1.6 | 2.10E−12 |
| 74 | PLOD2 | 531141 | 136714 | 0.257396812 | 1.6 | 5.11E−09 |
| 67 | NAP1L1 | 530626 | 131542 | 0.247899651 | 1.5 | 9.00E−06 |
| 90 | SFTPC | 530239 | 130739 | 0.246566171 | 1.5 | 2.04E−05 |
| 56 | KRT5 | 529486 | 126862 | 0.239594626 | 1.5 | 7.11E−04 |
| 98 | STC1 | 531825 | 123566 | 0.232343346 | 1.4 | 2.13E−04 |
| 68 | NFYB | 530432 | 121207 | 0.228506199 | 1.4 | 6.70E−02 |
| 33 | FADD | 530789 | 112595 | 0.212127606 | 1.3 | 1.00E−01 |
| 66 | MYLK | 530197 | 111609 | 0.210504775 | 1.3 | 1.03E−01 |
| 1 | ACTA2 | 529611 | 110425 | 0.208502089 | 1.3 | 1.09E−01 |
| 14 | CD79A | 530466 | 110121 | 0.207592947 | 1.3 | 1.35E−01 |
| 57 | KTN1 | 531003 | 103625 | 0.195149557 | 1.2 | 2.10E−01 |
| 101 | THBD | 531528 | 99764 | 0.18769284 | 1.1 | 2.49E−01 |
| 88 | SERPIND1 | 529983 | 97979 | 0.184871968 | 1.1 | 2.51E−01 |
| 49 | IGJ | 531073 | 97815 | 0.184183719 | 1.1 | 0.278 |
| 72 | PCSK1 | 531081 | 97054 | 0.182748018 | 1.1 | 0.28 |
| 80 | RET | 531418 | 95402 | 0.179523464 | 1.1 | 0.291 |
| 50 | IL6ST | 530372 | 94286 | 0.177773336 | 1.1 | 0.293 |
| 26 | CTNND1 | 531448 | 92494 | 0.174041487 | 1.1 | 0.295 |
| 54 | KIAA1128 | 530302 | 92462 | 0.174357253 | 1.1 | 0.295 |
| 85 | SELL | 530381 | 92229 | 0.173891976 | 1.1 | 0.296 |
| 25 | CSTB | 530302 | 91993 | 0.173472851 | 1.1 | 0.297 |
| 42 | GRB7 | 530720 | 90789 | 0.171067606 | 1.0 | 0.299 |
| 91 | SLC1A6 | 531445 | 90768 | 0.17079472 | 1.0 | 0.299 |
| 34 | FEZ2 | 530668 | 89237 | 0.168159753 | 1.0 | 0.321 |
| 84 | SCNN1A | 530854 | 88757 | 0.16719663 | 1.0 | 0.333 |
| 9 | CALB2 | 530704 | 87965 | 0.16575153 | 1.0 | 0.335 |
| 45 | HSP90B1 | 531592 | 87510 | 0.16461873 | 1.0 | 0.38 |
| 27 | DDC | 531607 | 87490 | 0.164576463 | 1.0 | 0.381 |
| 18 | CNN1 | 531402 | 87280 | 0.164244771 | 1.0 | 0.385 |
| 11 | CASP4 | 531535 | 86217 | 0.162203806 | 1.0 | 0.4 |
| 19 | CNN3 | 530197 | 85014 | 0.160344174 | 1.0 | 0.405 |
| 78 | RBM5 | 531363 | 84993 | 0.159952801 | 1.0 | 0.466 |
| 5 | ARCN1 | 530675 | 84744 | 0.15969096 | 1.0 | 0.474 |
| 48 | IGFBP3 | 531841 | 83933 | 0.157815964 | 1.0 | 0.485 |
| 94 | SNRPB | 531941 | 83130 | 0.15627673 | 1.0 | 0.5 |
| 92 | SLC20A1 | 530870 | 82837 | 0.156040085 | 1.0 | 0.5 |

1. A method of prognosing or classifying a subject with non-small cell lung cancer (NSCLC) comprising:
(a) determining the expression of at least three biomarkers in a test sample from the subject selected from CALCA, CCR7, STX1A, CCT3, SPRR1B, SELP, PAFAH1B3, CPE, XRCC6, HIF1A, MARCH6, PLOD2, NAP1L1, SFTPC, KRT5 and STC1; and
(b) comparing expression of the at least three biomarkers in the test sample with expression of the at least three biomarkers in a control sample;
wherein a difference or similarity in the expression of the at least three biomarkers between the control and the test sample is used to prognose or classify the subject with NSCLC into a poor survival group or a good survival group.

2.-3. (canceled)

4. The method claim 1, wherein the at least three biomarkers are selected from CALCA, CCR7, STX1A, CCT3, SPRR1B, SELP, PAFAH1B3, CPE, XRCC6, HIF1A, MARCH6, PLOD2, NAP1L1, SFTPC and KRT5.

5. The method of claim 1, wherein the at least three biomarkers is three biomarkers.

6. The method of claim 1, wherein the at least three biomarkers is four biomarkers.

7. The method of claim 1, wherein the at least three biomarkers is five biomarkers.

8. The method of claim 1, wherein the at least three biomarkers is six biomarkers.

9. The method of claim 1, wherein the at least three biomarkers is seven biomarkers.

10. The method of claim 1, wherein the at least three biomarkers is eight biomarkers.

11. The method of claim 1, wherein the at least three biomarkers is nine biomarkers.

12. The method of claim 1, wherein the at least three biomarkers is ten biomarkers.

13. The method of claim 1, wherein the at least three biomarkers is eleven biomarkers.

14. The method of claim 1, wherein the at least three biomarkers is twelve biomarkers.

15. The method of claim 1, wherein the at least three biomarkers is thirteen biomarkers.

16. The method of claim 1, wherein the at least three biomarkers is fourteen biomarkers.

17. The method of claim 1, wherein the at least three biomarkers is fifteen biomarkers.

18. The method of claim 1, wherein the at least three biomarkers is sixteen biomarkers.

19. The method of claim 1, wherein the NSCLC is stage I or stage II.

20-24. (canceled)

25. A method of selecting a therapy for a subject with NSCLC, comprising the steps:
(c) classifying the subject with NSCLC into a poor survival group or a good survival group according to the method of claim 1; and
(d) selecting adjuvant chemotherapy for the poor survival group or no adjuvant chemotherapy for the good survival group.

26.-28. (canceled)

29. A computer program product for use in conjunction with a computer having a processor and a memory connected to the processor, the computer program product comprising a computer readable storage medium having a computer mechanism encoded thereon, wherein the computer program mechanism may be loaded into the memory of the computer and cause the computer to carry out the method of claim 1.

30.-53. (canceled)
